SEQUENCE LISTING (35 sequences)

SEQ ID NO: 1 - protein, 110 aa, Artificial Sequence
  VLCD3 Domain of DART-A
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLG

SEQ ID NO: 2 - protein, 14 aa, Artificial Sequence
  CDR1 of the VLCD3 Domain of DART-A
RSSTGAVTTSNYAN

SEQ ID NO: 3 - protein, 7 aa, Artificial Sequence
  CDR2 of the VLCD3 Domain of DART-A
GTNKRAP

SEQ ID NO: 4 - protein, 9 aa, Artificial Sequence
  CDR2 of the VLCD3 Domain of DART-A
ALWYSNLWV

SEQ ID NO: 5 - protein, 8 aa, Artificial Sequence
  Linker 1
GGGSGGGG

SEQ ID NO: 6 - protein, 120 aa, Artificial Sequence
  VHCD123 Domain of DART-A
EVQLVQSGAELKKPGASVKVSCKASGYTFTDYYMKWVRQAPGQGLEWIGDIIPSNGATFYNQKFKGRVTITVDKSTSTAYMELSSLRSEDTAVYYCARSHLLRASWFAYWGQGTLVTVSS

SEQ ID NO: 7 - protein, 5 aa, Artificial Sequence
  CDR1 of the VHCD123 Domain of DART-A
DYYMK

SEQ ID NO: 8 - protein, 17 aa, Artificial Sequence
  CDR2 of the VHCD123 Domain of DART-A
DIIPSNGATFYNQKFKG

SEQ ID NO: 9 - protein, 11 aa, Artificial Sequence
  CDR3 of the VHCD123 Domain of DART-A
SHLLRASWFAY

SEQ ID NO: 10 - protein, 113 aa, Artificial Sequence
  VLCD123 Domain of DART-A
DFVMTQSPDSLAVSLGERVTMSCKSSQSLLNSGNQKNYLTWYQQKPGQPPKLLIYWASTRESGVPDRFSGSGSGTDFTLTISSLQAEDVAVYYCQNDYSYPYTFGQGTKLEIK

SEQ ID NO: 11 - protein, 17 aa, Artificial Sequence
  CDR1 of the VLCD123 Domain of DART-A
KSSQSLLNSGNQKNYLT

SEQ ID NO: 12 - protein, 7 aa, Artificial Sequence
  CDR2 of the VLCD123 Domain of DART-A
WASTRES

SEQ ID NO: 13 - protein, 9 aa, Artificial Sequence
  CDR3 of the VLCD123 Domain of DART-A
QNDYSYPYT

SEQ ID NO: 14 - protein, 125 aa, Artificial Sequence
  VHCD3 Domain of DART-A
EVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSS

SEQ ID NO: 15 - protein, 5 aa, Artificial Sequence
  CDR1 of the VHCD3 Domain of DART-A
TYAMN

SEQ ID NO: 16 - protein, 19 aa, Artificial Sequence
  CDR2 of the VHCD3 Domain of DART-A
RIRSKYNNYATYYADSVKD

SEQ ID NO: 17 - protein, 14 aa, Artificial Sequence
  CDR3 of the VHCD3 Domain of DART-A
HGNFGNSYVSWFAY

SEQ ID NO: 18 - protein, 6 aa, Artificial Sequence
  Linker 2
GGCGGG

SEQ ID NO: 19 - protein, 28 aa, Artificial Sequence
  Heterodimer-Promoting “E-Coil” Domain
EVAALEKEVAALEKEVAALEKEVAALEK

SEQ ID NO: 20 - protein, 28 aa, Artificial Sequence
  Heterodimer-Promoting “K-Coil” Domain
KVAALKEKVAALKEKVAALKEKVAALKE

SEQ ID NO: 21 - protein, 272 aa, Artificial Sequence
  First Polypeptide of DART-A
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGEVQLVQSGAELKKPGASVKVSCKASGYTFTDYYMKWVRQAPGQGLEWIGDIIPSNGATFYNQKFKGRVTITVDKSTSTAYMELSSLRSEDTAVYYCARSHLLRASWFAYWGQGTLVTVSSGGCGGGEVAALEKEVAALEKEVAALEKEVAALEK

SEQ ID NO: 22 - DNA, 816 bp, Artificial Sequence
  Polynucleotide Encoding First Polypeptide Chain of DART-A
caggctgtggtgactcaggagccttcactgaccgtgtccccaggcggaactgtgaccctgacatgcagatccagcacaggcgcagtgaccacatctaactacgccaattgggtgcagcagaagccaggacaggcaccaaggggcctgatcgggggtacaaacaaaagggctccctggacccctgcacggttttctggaagtctgctgggcggaaaggccgctctgactattaccggggcacaggccgaggacgaagccgattactattgtgctctgtggtatagcaatctgtgggtgttcgggggtggcacaaaactgactgtgctgggagggggtggatccggcggcggaggcgaggtgcagctggtgcagtccggggctgagctgaagaaacccggagcttccgtgaaggtgtcttgcaaagccagtggctacaccttcacagactactatatgaagtgggtcaggcaggctccaggacagggactggaatggatcggcgatatcattccttccaacggggccactttctacaatcagaagtttaaaggcagggtgactattaccgtggacaaatcaacaagcactgcttatatggagctgagctccctgcgctctgaagatacagccgtgtactattgtgctcggtcacacctgctgagagccagctggtttgcttattggggacagggcaccctggtgacagtgtcttccggaggatgtggcggtggagaagtggccgcactggagaaagaggttgctgctttggagaaggaggtcgctgcacttgaaaaggaggtcgcagccctggagaaa

SEQ ID NO: 23 - protein, 280 aa, Artificial Sequence
  Second Polypeptide Chain of DART-A
DFVMTQSPDSLAVSLGERVTMSCKSSQSLLNSGNQKNYLTWYQQKPGQPPKLLIYWASTRESGVPDRFSGSGSGTDFTLTISSLQAEDVAVYYCQNDYSYPYTFGQGTKLEIKGGGSGGGGEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSSGGCGGGKVAALKEKVAALKEKVAALKEKVAALKE

SEQ ID NO: 24 - DNA, 840 bp, Artificial Sequence
  Polynucleotide Encoding Second Polypeptide Chain of DART-A
gacttcgtgatgacacagtctcctgatagtctggccgtgagtctgggggagcgggtgactatgtcttgcaagagctcccagtcactgctgaacagcggaaatcagaaaaactatctgacctggtaccagcagaagccaggccagccccctaaactgctgatctattgggcttccaccagggaatctggcgtgcccgacagattcagcggcagcggcagcggcacagattttaccctgacaatttctagtctgcaggccgaggacgtggctgtgtactattgtcagaatgattacagctatccctacactttcggccaggggaccaagctggaaattaaaggaggcggatccggcggcggaggcgaggtgcagctggtggagtctgggggaggcttggtccagcctggagggtccctgagactctcctgtgcagcctctggattcaccttcagcacatacgctatgaattgggtccgccaggctccagggaaggggctggagtgggttggaaggatcaggtccaagtacaacaattatgcaacctactatgccgactctgtgaaggatagattcaccatctcaagagatgattcaaagaactcactgtatctgcaaatgaacagcctgaaaaccgaggacacggccgtgtattactgtgtgagacacggtaacttcggcaattcttacgtgtcttggtttgcttattggggacaggggacactggtgactgtgtcttccggaggatgtggcggtggaaaagtggccgcactgaaggagaaagttgctgctttgaaagagaaggtcgccgcacttaaggaaaaggtcgcagccctgaaagag

SEQ ID NO: 25 - protein, 288 aa, Homo sapiens
  MISC_FEATURE (1)..(298): Human PD-1 polypeptide (NCBI NP_005009.2)
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 26 - protein, 119 aa, Artificial Sequence
  VH Domain of Anti-PD1 Antibody PD-1 mAb 1
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSS

SEQ ID NO: 27 - protein, 111 aa, Artificial Sequence
  VL Domain of Anti-PD1 Antibody PD-1 mAb 1
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIK

SEQ ID NO: 28 - protein, 176 aa, Homo sapiens
  MISC_FEATURE (1)..(175): Human B7-H1 (PD-L1) Polypeptide (NCBI NP_001254635.1)
MRIFAVFIFMTYWHLLNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 29 - protein, 273 aa, Homo sapiens
  MISC_FEATURE (1)..(273): Human B7-DC (PD-L2) Polypeptide (NCBI NP_079515.2)
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 30 - protein, 107 aa, Homo sapiens [sequence omitted]

SEQ ID NO: 31 - protein, 98 aa, Homo sapiens
  MISC_FEATURE (1)..(98): Human IgG4 CH1 Domain
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 32 - protein, 12 aa, Artificial Sequence
  IgG4 Hinge Variant Comprising a Stabilizing S228P Substitution
ESKYGPPCPPCP

SEQ ID NO: 33 - protein, 217 aa, Homo sapiens
  MISC_FEATURE (1)..(217): CH2-CH3 Domain of Human IgG4
  MISC_FEATURE (217)..(217): AXX is Lysine (K) or is Absent
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGX

SEQ ID NO: 34 - protein, 445 aa, Artificial Sequence
  Heavy Chain of anti-PD-1 Antibody mAb1 IgG4
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 35 - protein, 218 aa, Artificial Sequence
  Light Chain of anti-PD-1 Antibody mAb1 IgG4
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2022/0213203 A1
(43) Pub. Date: Jul. 7, 2022

(54) Dosing Regimens of Bispecific CD123 x CD3 Diabodies in the Treatment of Hematologic Malignancies

(71) Applicant: MacroGenics, Inc., Rockville, MD (US)

(72) Inventors: Jan Kenneth Davidson, Rockville, MD (US); Ian Lent, Rockville, MD (US); Krishnan Sampathkumar, Rockville, MD (US); Ralph Froman Alderson, Rockville, MD (US); Ross La Motte-Mohs, Rockville, MD (US); Jon Marc Wigginton, Rockville, MD (US)

(73) Assignee: MacroGenics, Inc., Rockville, MD (US)

(21) Appl. No.: 17/601,787

(22) PCT Filed: Apr. 8, 2020
(86) PCT No.: PCT/US2020/027140

§ 371 Date: Oct. 6, 2021

Related U.S. Application Data
(60) Provisional application No. 62/831,979, filed on Apr. 10, 2019.
(60) Provisional application No. 62/831,969, filed on Apr. 10, 2019.
(60) Provisional application No. 62/929,401, filed on Nov. 1, 2019.
(60) Provisional application No. 62/929,381, filed on Nov. 1, 2019.
(60) Provisional application No. 63/001,388, filed on Mar. 29, 2020.

(51) Int. Cl. C07K 16/28; A61P 35/00; A61K 47/06; A61K 39/395; A61P 35/02

(52) U.S. Cl. CPC C07K 16/2866; A61P 35/00; C07K 16/2809; A61K 47/06; A61K 2039/505; A61P 35/02; C07K 16/2818; C07K 2317/31; A61K 39/3955

(57) ABSTRACT

The present invention is directed to a dosing regimen for administering a CD 123×CDS bispecific diabody to patients with a hematologic malignancy such as acute myeloid leukemia (AML) or myelodysplastic syndrome (MDS). The present invention is also directed to a dosing regimen for administering a CD 123×CDS bispecific diabody in combination with a molecule capable of binding PD-1 or a natural ligand of PD-1 (a “PD-1 or PD-1 ligand binding molecule”) to patients with a hematologic malignancy such as acute myeloid leukemia (AML) or myelodysplastic syndrome (MDS). The invention particularly concerns the use of such regimens for administering the sequence-optimized CD 123×CDS bispecific diabody, “DART-A,” which is capable of simultaneous binding to CD 123 and CDS.

### CROSS REFERENCE TO RELATED APPLICATIONS

This application claims priority to U.S. patent application Ser. Nos. 63/001,388 (filed on Mar. 29, 2020, pending), 62/831,969 (filed on Apr. 10, 2019; pending); 62/831,979 (filed on Apr. 10, 2019; pending); 62/929,381 (filed on Nov. 1, 2019; pending); and 62/929,401 (filed on Nov. 1, 2019; pending), each of which applications are herein incorporated by reference in their entirety.

### REFERENCE TO SEQUENCE LISTING

This application includes one or more Sequence Listings pursuant to 37 C.F.R. 1.821 et seq., which are disclosed in computer-readable media (file name: 1301_0162P3_PCT_ST25.txt, created on Mar. 29, 2020, and having a size of 35,519 bytes), which file is incorporated herein in its entirety.

### FIELD OF THE INVENTION

The present invention is directed to a dosing regimen for administering a CD123×CD3 bispecific diabody to patients with a hematologic malignancy such as acute myeloid leukemia (AML) or myelodysplastic syndrome (MDS). The present invention is also directed to a dosing regimen for administering a CD123×CD3 bispecific diabody in combination with a molecule capable of binding PD-1 or a natural ligand of PD-1 (a “PD-1 or PD-1 ligand binding molecule”) to patients with a hematologic malignancy such as acute myeloid leukemia (AML) or myelodysplastic syndrome (MDS). The invention particularly concerns the use of such regimens for the sequence-optimized CD123×CD3 bispecific diabody “DART-A,” that is capable of simultaneous binding to CD123 and CD3.

### BACKGROUND OF THE INVENTION

### I. AML and MDS

AML and MDS are thought to arise in and be perpetuated by a small population of leukemic stem cells (LSCs), which are generally dormant (i.e., not rapidly dividing cells) and therefore resist cell death (apoptosis) and conventional chemotherapeutic agents. LSCs are characterized by high levels of CD123 expression, which are not present in the corresponding normal hematopoietic stem cell population in normal human bone marrow (Jin, W. et al. (2009) “Regulation Of Th17 Cell Differentiation And EAE Induction By MAP3K NIK,” Blood 113:6603-6610; Jordan, C. T. et al. (2000) “The Interleukin-3 Receptor Alpha Chain Is A Unique Marker For Human Acute Myelogenous Leukemia Stem Cells,” Leukemia 14:1777-1784). CD123 is expressed in 45%-95% of AML, 85% of Hairy cell leukemia (HCL), and 40% of acute B lymphoblastic leukemia (B-ALL). CD123 expression is also associated with multiple other malignancies/pre-malignancies: chronic myeloid leukemia (CML) progenitor cells (including blast crisis CML); Hodgkin's Reed Sternberg (RS) cells; transformed non-Hodgkin's lymphoma (NHL); some chronic lymphocytic leukemia (CLL) (CD11c+); a subset of acute T lymphoblastic leukemia (T-ALL) (16%, most immature, mostly adult), plasmacytoid dendritic cell (pDC) (DC2) malignancies and CD34+/CD38− myelodysplastic syndrome (MDS) marrow cell malignancies.

AML is a clonal disease characterized by the proliferation and accumulation of transformed myeloid progenitor cells in the bone marrow, which ultimately leads to hematopoietic failure. The incidence of AML increases with age, and older patients typically have worse treatment outcomes than do younger patients (Robak, T. et al. (2009) “Current And Emerging Therapies For Acute Myeloid Leukemia,” Clin. Ther. 2:2349-2370). Unfortunately, at present, most adults with AML die from their disease.

Treatment for AML initially focuses in the induction of remission (induction therapy). Once remission is achieved, treatment shifts to focus on securing such remission (post-remission or consolidation therapy) and, in some instances, maintenance therapy. The standard remission induction paradigm for AML is chemotherapy with an anthracycline/cytarabine combination, followed by either consolidation chemotherapy (usually with higher doses of the same drugs as were used during the induction period) or human hematopoietic stem cell transplantation (HSCT), depending on the patient's ability to tolerate intensive treatment and the likelihood of cure with chemotherapy alone (see, e.g., Roboz, G. J. (2012) “Current Treatment Of Acute Myeloid Leukemia,” Curr. Opin. Oncol. 24:711-719).

Agents frequently used in induction therapy include cytarabine and the anthracyclines. Cytarabine, also known as AraC, kills cancer cells (and other rapidly dividing normal cells) by interfering with DNA synthesis. Side effects associated with AraC treatment include decreased resistance to infection, a result of decreased white blood cell production; bleeding, as a result of decreased platelet production; and anemia, due to a potential reduction in red blood cells. Other side effects include nausea and vomiting. Anthracyclines (e.g., daunorubicin, doxorubicin, and idarubicin) have several modes of action including inhibition of DNA and RNA synthesis, disruption of higher order structures of DNA, and production of cell damaging free oxygen radicals. The most consequential adverse effect of anthracyclines is cardiotoxicity, which considerably limits administered life-time dose and to some extent their usefulness.

Thus, unfortunately, despite substantial progress in the treatment of newly diagnosed AML, 20% to 40% of patients do not achieve remission with the standard induction chemotherapy, and 50% to 70% of patients entering a first complete remission are expected to relapse within 3 years. The optimum strategy at the time of relapse, or for patients with the resistant disease, remains uncertain. Stem cell transplantation has been established as the most effective form of anti-leukemic therapy in patients with AML in first or subsequent remission (Roboz, G. J. (2012) “Current Treatment Of Acute Myeloid Leukemia,” Curr. Opin. Oncol. 24:711-719).

### II. CD123

CD123 (interleukin 3 receptor alpha, IL-3Rα) is a 40 kDa molecule and is part of the interleukin 3 receptor complex (Stomski, F. C. et al. (1996) “Human Interleukin-3 (IL-3) Induces Disulfide-Linked IL-3 Receptor Alpha-And Beta-Chain Heterodimerization, Which Is Required For Receptor Activation But Not High-Affinity Binding,” Mol. Cell. Biol. 16(6):3035-3046). Interleukin 3 (IL-3) drives early differentiation of multipotent stem cells into cells of the erythroid, myeloid and lymphoid progenitors. CD123 is expressed on CD34+ committed progenitors (Taussig, D. C. et al. (2005) “Hematopoietic Stem Cells Express Multiple Myeloid Markers: Implications For The Origin And Targeted Therapy Of Acute Myeloid Leukemia,” Blood 106:4086-4092), but not by CD34+/CD38− normal hematopoietic stem cells. CD123 is expressed by basophils, mast cells, plasmacytoid dendritic cells, some expression by monocytes, macrophages and eosinophils, and low or no expression by neutrophils and megakaryocytes. Some non-hematopoietic tissues (placenta, Leydig cells of the testis, certain brain cell elements and some endothelial cells) express CD123; however, expression is mostly cytoplasmic.

CD123 is reported to be expressed by leukemic blasts and leukemia stem cells (LSC) (Jordan, C. T. et al. (2000) “The Interleukin-3 Receptor Alpha Chain Is A Unique Marker For Human Acute Myelogenous Leukemia Stem Cells,” Leukemia 14:1777-1784; Jin, W. et al. (2009) “Regulation Of Th17 Cell Differentiation And EAE Induction By MAP3K NIK,” Blood 113:6603-6610). In human normal precursor populations, CD123 is expressed by a subset of hematopoietic progenitor cells (HPC) but not by normal hematopoietic stem cells (HSC). CD123 is also expressed by plasmacytoid dendritic cells (pDC) and basophils, and, to a lesser extent, monocytes and eosinophils (Lopez, A. F. et al. (1989) “Reciprocal Inhibition Of Binding Between Interleukin 3 And Granulocyte-Macrophage Colony-Stimulating Factor To Human Eosinophils,” Proc. Natl. Acad. Sci. (U.S.A.) 86:7022-7026; Sun, Q. et al. (1996) “Monoclonal Antibody 7G3 Recognizes The N-Terminal Domain Of The Human Interleukin-3 (IL-3) Receptor Alpha Chain And Functions As A Specific IL-3 Receptor Antagonist,” Blood 87:83-92; Muñoz, L. et al. (2001) “Interleukin-3 Receptor Alpha Chain (CD123) Is Widely Expressed In Hematologic Malignancies,” Haematologica 86(12):1261-1269; Masten, B. J. et al. (2006) “Characterization Of Myeloid And Plasmacytoid Dendritic Cells In Human Lung,” J. Immunol. 177:7784-7793; Korpelainen, E. I. et al. (1995) “Interferon-Gamma Upregulates Interleukin-3 (IL-3) Receptor Expression In Human Endothelial Cells And Synergizes With IL-3 In Stimulating Major Histocompatibility Complex Class II Expression And Cytokine Production,” Blood 86:176-182).

CD123 has been reported to be overexpressed on malignant cells in a wide range of hematologic malignancies including acute myeloid leukemia (AML) and myelodysplastic syndrome (MDS) (Munoz, L. et al. (2001) “Interleukin-3 Receptor Alpha Chain (CD123) Is Widely Expressed In Hematologic Malignancies,” Haematologica 86(12):1261-1269). Overexpression of CD123 is associated with poorer prognosis in AML (Tettamanti, M. S. et al. (2013) “Targeting Of Acute Myeloid Leukaemia By Cytokine-Induced Killer Cells Redirected With A Novel CD123-Specific Chimeric Antigen Receptor,” Br. J. Haematol. 161:389-401).

### III. CD3

CD3 is a T cell co-receptor composed of four distinct chains (Wucherpfennig, K. W. et al. (2010) “Structural Biology Of The T-Cell Receptor: Insights Into Receptor Assembly, Ligand Recognition, And Initiation Of Signaling,” Cold Spring Harb. Perspect. Biol. 2(4):a005140; pages 1-14). In mammals, the complex contains a CD3γ chain, a CD3δ chain, and two CD3ε chains. These chains associate with a molecule known as the T cell receptor (TCR) in order to generate an activation signal in T lymphocytes. In the absence of CD3, TCRs do not assemble properly and are degraded (Thomas, S. et al. (2010) “Molecular Immunology Lessons From Therapeutic T-Cell Receptor Gene Transfer,” Immunology 129(2):170-177). CD3 is found bound to the membranes of all mature T cells, and in virtually no other cell type (see, Janeway, C. A. et al. (2005) In: IMMUNOBIOLOGY: THE IMMUNE SYSTEM IN HEALTH AND DISEASE,” 6th ed. Garland Science Publishing, NY, pp. 214-216; Sun, Z. J. et al. (2001) “Mechanisms Contributing To T Cell Receptor Signaling And Assembly Revealed By The Solution Structure Of An Ectodomain Fragment Of The CD3ε:γ Heterodimer,” Cell 105(7):913-923; Kuhns, M. S. et al. (2006) “Deconstructing The Form And Function Of The TCR/CD3 Complex,” Immunity. 2006 February; 24(2):133-139).

### IV. The Programmed Death-1 (“PD-1”) Membrane Protein

Programmed Death-1 (“PD-1,” also known as “CD279”) is an approximately 31 kD type I membrane protein member of the extended CD28/CTLA4 family of T-cell regulators that broadly negatively regulates immune responses (Ishida, Y. et al. (1992) “Induced Expression Of PD-1, A Novel Member Of The Immunoglobulin Gene Superfamily, Upon Programmed Cell Death,” EMBO J. 11:3887-3895; United States Patent Application Publication No. 2007/0202100; 2008/0311117; 2009/00110667; U.S. Pat. Nos. 6,808,710; 7,101,550; 7,488,802; 7,635,757; 7,722,868; PCT Publication No. WO 01/14557).

PD-1 is expressed on activated T-cells, B-cells, and monocytes (Agata, Y. et al. (1996) “Expression Of The PD-1 Antigen On The Surface Of Stimulated Mouse T And B Lymphocytes,” Int. Immunol. 8(5):765-772; Yamazaki, T. et al. (2002) “Expression Of Programmed Death 1 Ligands By Murine T-Cells And APC,” J. Immunol. 169:5538-5545) and at low levels in natural killer (NK) T-cells (Nishimura, H. et al. (2000) “Facilitation Of Beta Selection And Modification Of Positive Selection In The Thymus Of PD-1-Deficient Mice,” J. Exp. Med. 191:891-898; Martin-Orozco, N. et al. (2007) “Inhibitory Costimulation And Anti-Tumor Immunity,” Semin. Cancer Biol. 17(4):288-298).

PD-1 mediates its inhibition of the immune system by binding B7-H1 and B7-DC (also known as PD-L1 and PD-L2) (Flies, D. B. et al. (2007) “The New B7s: Playing a Pivotal Role in Tumor Immunity,” J. Immunother. 30(3):251-260; U.S. Pat. Nos. 6,803,192; 7,794,710; United States Patent Application Publication Nos. 2005/0059051; 2009/0055944; 2009/0274666; 2009/0313687; PCT Publication Nos. WO 01/39722; WO 02/086083).

B7-H1 and B7-DC are broadly expressed on the surfaces of many types of human and murine tissues, such as heart, placenta, muscle, fetal liver, spleen, lymph nodes, and thymus as well as murine liver, lung, kidney, islets cells of the pancreas and small intestine (Martin-Orozco, N. et al. (2007) “Inhibitory Costimulation And Anti-Tumor Immunity,” Semin. Cancer Biol. 17(4):288-298). In humans, B7-H1 protein expression has been found in human endothelial cells (Chen, Y. et al. (2005) “Expression of B7-H1 in Inflammatory Renal Tubular Epithelial Cells,” Nephron. Exp. Nephrol. 102:e81-e92; de Haij, S. et al. (2005) “Renal Tubular Epithelial Cells Modulate T-Cell Responses Via ICOS-L And B7-H1” Kidney Int. 68:2091-2102; Mazanet, M. M. et al. (2002) “B7-H1 Is Expressed By Human Endothelial Cells And Suppresses T-Cell Cytokine Synthesis,” J. Immunol. 169:3581-3588), myocardium (Brown, J. A. et al. (2003) “Blockade Of Programmed Death-1 Ligands On Dendritic Cells Enhances T-Cell Activation And Cytokine Production,” J. Immunol. 170:1257-1266), and syncyciotrophoblasts (Petroff, M. G. et al. (2002) “B7 Family Molecules: Novel Immunomodulators At The Maternal-Fetal Interface,” Placenta 23:S95-S101). The molecules are also expressed by resident macrophages of some tissues, by macrophages that have been activated with interferon (IFN)-γ or tumor necrosis factor (TNF)-α (Latchman, Y. et al. (2001) “PD-L2 Is A Second Ligand For PD-1 And Inhibits T-Cell Activation,” Nat. Immunol 2:261-268), and in tumors (Dong, H. (2003) “B7-H1 Pathway And Its Role In The Evasion Of Tumor Immunity,” J. Mol. Med. 81:281-287).

The interaction between B7-H1 and PD-1 has been found to provide a crucial negative costimulatory signal to T- and B-cells (Martin-Orozco, N. et al. (2007) “Inhibitory Costimulation And Anti-Tumor Immunity,” Semin. Cancer Biol. 17(4):288-298) and functions as a cell death inducer (Ishida, Y. et al. (1992) “Induced Expression Of PD-1, A Novel Member Of The Immunoglobulin Gene Superfamily, Upon Programmed Cell Death,” EMBO J. 11:3887-3895; Subudhi, S. K. et al. (2005) “The Balance Of Immune Responses: Costimulation Verse Coinhibition,” J. Molec. Med. 83:193-202). More specifically, interaction between low concentrations of the PD-1 receptor and the B7-H1 ligand has been found to result in the transmission of an inhibitory signal that strongly inhibits the proliferation of antigen-specific CD8⁺ T-cells; at higher concentrations the interactions with PD-1 do not inhibit T-cell proliferation but markedly reduce the production of multiple cytokines (Sharpe, A. H. et al. (2002) “The B7-CD28 Superfamily,” Nature Rev. Immunol. 2:116-126). T-cell proliferation and cytokine production by both resting and previously activated CD4 and CD8 T-cells, and even naive T-cells from umbilical-cord blood, have been found to be inhibited by soluble B7-H1-Fc fusion proteins (Freeman, G. J. et al. (2000) “Engagement Of The PD-1 Immunoinhibitory Receptor By A Novel B7 Family Member Leads To Negative Regulation Of Lymphocyte Activation,” J. Exp. Med. 192:1-9; Latchman, Y. et al. (2001) “PD-L2 Is A Second Ligand For PD-1 And Inhibits T-Cell Activation,” Nature Immunol. 2:261-268; Carter, L. et al. (2002) “PD-1:PD-L Inhibitory Pathway Affects Both CD4(+) and CD8(+) T-cells And Is Overcome By IL-2,” Eur. J. Immunol. 32(3):634-643; Sharpe, A. H. et al. (2002) “The B7-CD28 Superfamily,” Nature Rev. Immunol. 2:116-126).

Molecules (e.g., antibodies, etc.) that bind to PD-1 and impede its ability to bind to its natural ligands thus inhibit the ability of PD-1 to inhibit the immune system; such molecules thus promote an active immune response. Conversely, molecules (e.g., antibodies, etc.) that bind to a natural ligand of PD-1 (especially B7-H1) and impede its ability to bind PD-1, inhibit the ability of PD-1 to inhibit the immune system; such molecules thus also promote an active immune response.

The role of B7-H1 and PD-1 in inhibiting T-cell activation and proliferation has thus suggested that these biomolecules might serve as therapeutic targets for treatments of inflammation and cancer. Thus, the use of PD-1 or PD-L1 binding molecules such as anti-PD-1 and anti-B7-H1 antibodies to treat infections and tumors and up-modulate an adaptive immune response has been proposed (see e.g., Nishijima, T. F., et al. (2017) “Safety and Tolerability of PD-1/PD-L1 Inhibitors Compared with Chemotherapy in Patients with Advanced Cancer: A Meta-Analysis,” The oncologist 22(4):470-479; Rao, M., et al. (2017) “Anti-PD-1/PD-L1 therapy for infectious diseases: learning from the cancer paradigm,” Intnl. J. of Infect. Dis. 56:221-228). Antibodies capable of specifically binding to PD-1 and B7-H1 have been described (see, e.g., Tables 3-4).

### V. Bispecific Diabodies

The provision of non-monospecific diabodies provides a significant advantage over monospecific natural antibodies: the capacity to co-ligate and co-localize cells that express different epitopes. Bispecific diabodies thus have wide-ranging applications including therapy and immunodiagnosis. Bispecificity allows for great flexibility in the design and engineering of the diabody in various applications, providing enhanced avidity to multimeric antigens, the cross-linking of differing antigens, and directed targeting to specific cell types relying on the presence of both target antigens. Of particular importance is the co-ligating of differing cells, for example, the cross-linking of effector cells, such as cytotoxic T cells and tumor cells (Staerz et al. (1985) “Hybrid Antibodies Can Target Sites For Attack By T Cells,” Nature 314:628-631, and Holliger et al. (1996) “Specific Killing Of Lymphoma Cells By Cytotoxic T-Cells Mediated By A Bispecific Diabody,” Protein Eng. 9:299-305). By cross-linking tumor and effector cells, the diabody not only brings the effector cell within the proximity of the tumor cells, but leads to effective tumor killing (see e.g., Cao et al. (2003) “Bispecific Antibody Conjugates In Therapeutics,” Adv. Drug. Deliv. Rev. 55:171-197).

The formation of such non-monospecific diabodies requires the successful assembly of two or more distinct and different polypeptides (i.e., such formation requires that the diabodies be formed through the heterodimerization of different polypeptide chain species). This fact is in contrast to mono-specific diabodies, which are formed through the homodimerization of identical polypeptide chains. Because at least two dissimilar polypeptides (i.e., two polypeptide species) must be provided in order to form a non-monospecific diabody, and because homodimerization of such polypeptides leads to inactive molecules (Takemura, S. et al. (2000) “Construction Of A Diabody (Small Recombinant Bispecific Antibody) Using A Refolding System,” Protein Eng. 13(8):583-588), the production of such polypeptides must be accomplished in such a way as to prevent covalent bonding between polypeptides of the same species (i.e., so as to prevent homodimerization) (Takemura, S. et al. (2000) “Construction Of A Diabody (Small Recombinant Bispecific Antibody) Using A Refolding System,” Protein Eng. 13(8):583-588). The art has therefore taught the non-covalent association of such polypeptides (see, e.g., Olafsen et al. (2004) “Covalent Disulfide-Linked Anti-CEA Diabody Allows Site-Specific Conjugation And Radiolabeling For Tumor Targeting Applications,” Prot. Engr. Des. Sel. 17:21-27; Asano et al. (2004) “A Diabody For Cancer Immunotherapy And Its Functional Enhancement By Fusion Of Human Fc Domain,” Abstract 3P-683, J. Biochem. 76(8):992; Takemura, S. et al. (2000) “Construction Of A Diabody (Small Recombinant Bispecific Antibody) Using A Refolding System,” Protein Eng. 13 (8): 583-588; Lu, D. et al. (2005) “A Fully Human Recombinant IgG-Like Bispecific Antibody To Both The Epidermal Growth Factor Receptor And The Insulin-Like Growth Factor Receptor For Enhanced Antitumor Activity,” J. Biol. Chem. 280(20):19665-19672).

Bispecific diabodies composed of non-covalently associated polypeptides are unstable and readily dissociate into non-functional monomers (see, e.g., Lu, D. et al. (2005) “A Fully Human Recombinant IgG-Like Bispecific Antibody To Both The Epidermal Growth Factor Receptor And The Insulin-Like Growth Factor Receptor For Enhanced Antitumor Activity,” J. Biol. Chem. 280(20):19665-19672). Stable, covalently bonded heterodimeric non-monospecific diabodies have been described (see, e.g., WO 2006/113665; WO/2008/157379; WO 2010/080538; WO 2012/018687; WO/2012/162068; Johnson, S. et al. (2010) “Effector Cell Recruitment With Novel Fv-Based Dual-Affinity Re-Targeting Protein Leads To Potent Tumor Cytolysis And In Vivo B-Cell Depletion,” J. Molec. Biol. 399(3):436-449; Veri, M. C. et al. (2010) “Therapeutic Control Of B Cell Activation Via Recruitment Of Fcgamma Receptor IIb (CD32B) Inhibitory Function With A Novel Bispecific Antibody Scaffold,” Arthritis Rheum. 62(7): 1933-1943; Moore, P. A. et al. (2011) “Application Of Dual Affinity Retargeting Molecules To Achieve Optimal Redirected T-Cell Killing Of B-Cell Lymphoma,” Blood 117(17):4542-4551). Such diabodies incorporate one or more cysteine residues into each of the employed polypeptide species. For example, the addition of a cysteine residue to the C-terminus of such constructs has been shown to allow disulfide bonding between the polypeptide chains, stabilizing the resulting heterodimer without interfering with the binding characteristics of the bivalent molecule.

Bispecific diabodies targeting CD123 and CD3 capable of mediating T cell redirected cell killing of CD123-expressing malignant cells have been described (see, e.g., WO 2015/026892). Notwithstanding such success, an unmet need remains to develop dosing regimens for the administration of CD123×CD3 bispecific diabodies for the treatment of hematological malignancies, particularly dosing regimens that minimize undesirable side effects including for example, cytokine release syndrome (“CRS”) and which stimulate the immune system. The present invention directly addresses this need and others, as described below.

### SUMMARY OF THE INVENTION

The present invention is directed to a dosing regimen for administering a CD123×CD3 bispecific diabody to patients with a hematologic malignancy such as acute myeloid leukemia (AML) or myelodysplastic syndrome (MDS). The present invention is also directed to a dosing regimen for administering a CD123×CD3 bispecific diabody in combination with a molecule capable of binding PD-1 or a natural ligand of PD-1 (a “PD-1 or PD-1 ligand binding molecule”) to patients with a hematologic malignancy such as acute myeloid leukemia (AML) or myelodysplastic syndrome (MDS). The invention particularly concerns the use of such regimens for the sequence-optimized CD123×CD3 bispecific diabody “DART-A,” that is capable of simultaneous binding to CD123 and CD3.

In detail, the invention provides a method of treating a hematologic malignancy comprising administering a CD123×CD3 binding molecule to a subject in need thereof wherein:
(I) the CD123×CD3 binding molecule is a diabody consisting of a first polypeptide chain having the amino acid sequence of SEQ ID NO:21 and a second polypeptide chain having the amino acid sequence of SEQ ID NO:23; and
(II) the method comprises an initial 7-day treatment period (I7DP), wherein:
   (A) on day 1 of the I7DP, the CD123×CD3 binding molecule is administered to the subject at a dosage of about 30 ng/kg/day by continuous intravenous infusion;
   (B) on day 2 of the I7DP, the CD123×CD3 binding molecule is administered to the subject at a dosage of about 60 ng/kg/day by continuous intravenous infusion;
   (C) on day 3 of the I7DP, the CD123×CD3 binding molecule is administered to the subject at a dosage of about 100 ng/kg/day by continuous infusion;
   (D) on day 4 of the I7DP, the CD123×CD3 binding molecule is administered to the subject at a dosage of about 200 ng/kg/day by continuous intravenous infusion;
   (E) on day 5 of the I7DP, the CD123×CD3 binding molecule is administered to the subject at a dosage of about 300 ng/kg/day by continuous intravenous infusion;
   (F) on day 6 of the I7DP, the CD123×CD3 binding molecule is administered to the subject at a dosage of from about 300 ng/kg/day to about 400 ng/kg/day by continuous intravenous infusion; and
   (G) on day 7 of the I7DP, the CD123×CD3 binding molecule is administered to the subject at a dosage of from about 300 ng/kg/day to about 500 ng/kg/day by continuous intravenous infusion.

The invention is additionally directed to a CD123×CD3 binding molecule for use in the treatment of a hematologic malignancy of a subject, wherein:
(I) the CD123×CD3 binding molecule is a diabody consisting of a first polypeptide chain having the amino acid sequence of SEQ ID NO:21 and a second polypeptide chain having the amino acid sequence of SEQ ID NO:23; and
(II) the use comprises a initial 7-Day treatment period (I7DP), wherein:
   (A) on day 1 of the I7DP, the CD123×CD3 binding molecule is administered to the subject at a dosage of about 30 ng/kg/day by continuous intravenous infusion;
   (B) on day 2 of the I7DP, the CD123×CD3 binding molecule is administered to the subject at a dosage of about 60 ng/kg/day by continuous intravenous infusion;
   (C) on day 3 of the I7DP, the CD123×CD3 binding molecule is administered to the subject at a dosage of about 100 ng/kg/day by continuous infusion;
   (D) on day 4 of the I7DP, the CD123×CD3 binding molecule is administered to the subject at a dosage of about 200 ng/kg/day by continuous intravenous infusion;
   (E) on day 5 of the I7DP, the CD123×CD3 binding molecule is administered to the subject at a dosage of about 300 ng/kg/day by continuous intravenous infusion;
   (F) on day 6 of the I7DP, the CD123×CD3 binding molecule is administered to the subject at a dosage of from about 300 ng/kg/day to about 400 ng/kg/day by continuous intravenous infusion; and
   (G) on day 7 of the I7DP, the CD123×CD3 binding molecule is administered to the subject at a dosage of from about 300 ng/kg/day to about 500 ng/kg/day by continuous intravenous infusion.

The invention is additionally directed to the embodiment of all of such above-indicated methods and uses, wherein the method or the use comprises one or more additional 7-Day treatment periods (A7DP), wherein on days 1-7 of each of the one or more A7DP(s), the CD123×CD3 binding molecule is administered to the subject at a dosage of from about 300 ng/kg/day to about 500 ng/kg/day by continuous intravenous infusion.

The invention is additionally directed to the embodiment of all of such above-indicated methods and uses, wherein on day 6, and day 7 of the I7DP, the CD123×CD3 binding molecule is administered to the subject at a dosage of about 300 ng/kg/day.

The invention is additionally directed to the embodiment of all of such above-indicated methods and uses, wherein, the on days 1-7 of at least one of the one or more A7DP(s), the CD123×CD3 binding molecule is administered to the subject at a dosage of about 300 ng/kg/day.

The invention is additionally directed to the embodiment of all of such above-indicated methods and uses, wherein on day 6 and day 7 of the I7DP, the CD123×CD3 binding molecule is administered to the subject at a dosage of about 400 ng/kg/day.

The invention is additionally directed to the embodiment of all of such above-indicated methods and uses, wherein on days 1-7 of at least one of the one or more A7DP(s), the CD123×CD3 binding molecule is administered to the subject at a dosage of about 400 ng/kg/day.

The invention is additionally directed to the embodiment of all of such above-indicated methods and uses, wherein on day 6 of the I7DP, the CD123×CD3 binding molecule is administered to the subject at a dosage of about 400 ng/kg/day, and on day 7 of the I7DP, the CD123×CD3 binding molecule is administered to the subject at a dosage of about 500 ng/kg/day.

The invention is additionally directed to the embodiment of all of such above-indicated methods and uses, wherein on days 1-7 of at least one of the one or more A7DP(s), the CD123×CD3 binding molecule is administered to the subject at a dosage of about 500 ng/kg/day.

The invention is additionally directed to the embodiment of all of such above-indicated methods and uses that comprise three A7DPs.

The invention is additionally directed to the embodiment of all of such above-indicated methods and uses that comprise an additional four, eight, twelve, sixteen, or twenty A7DPs.

The invention is additionally directed to the embodiment of all of such above-indicated methods and uses, wherein at least one of the one or more A7DPs is followed by one or more further 7-day treatment periods (F7DPs), wherein on days 1-4 of each of the one or more F7DPs the CD123×CD3 binding molecule is administered to the subject, and on days 5-7 of each of the one or more F7DPs the subject is not provided with the CD123×CD3 binding molecule.

The invention is additionally directed to the embodiment of all of such above-indicated methods and uses, wherein on days 1-4 of at least one of the one or more F7DPs, the CD123×CD3 binding molecule is administered to the subject at a dosage of about 300 ng/kg/day to about 500 ng/kg/day by continuous intravenous infusion.

The invention is additionally directed to the embodiment of all of such above-indicated methods and uses, wherein on days 1-4 of at least one of the one or more F7DPs, the CD123×CD3 binding molecule is administered to the subject at a dosage of about 300 ng/kg/day.

The invention is additionally directed to the embodiment of all of such above-indicated methods and uses, wherein on days 1-4 of at least one of the one or more F7DPs, the CD123×CD3 binding molecule is administered to the subject at a dosage of about 400 ng/kg/day.

The invention is additionally directed to the embodiment of all of such above-indicated methods and uses, wherein on days 1-4 of at least one of the one or more F7DPs, the CD123×CD3 binding molecule is administered to the subject at a dosage of about 500 ng/kg/day.

The invention is additionally directed to the embodiment of all of such above-indicated methods and uses that comprise four F7DPs.

The invention is additionally directed to the embodiment of all of such above-indicated methods and uses that comprise an additional four, eight, twelve, sixteen, or twenty of the F7DPs.

The invention is additionally directed to the embodiment of all of such above-indicated methods and uses further comprising administering a molecule capable of binding PD-1 or a natural ligand of PD-1, and wherein said molecule capable of binding PD-1 comprises an epitope-binding domain of an antibody that binds PD-1, and said molecule capable of binding a natural ligand of PD-1 comprises an epitope-binding domain of an antibody that binds a natural ligand of PD-1.

The invention is additionally directed to the embodiment of all of such above-indicated methods and uses, wherein the binding molecule capable of binding PD-1 or a natural ligand of PD-1 is administered once every two weeks (Q2W), once every three weeks (Q3W), or once every four weeks (Q4W).

The invention is additionally directed to the embodiment of all of such above-indicated methods and uses, wherein the binding molecule capable of binding PD-1 or a natural ligand of PD-1 is administered starting on day 15.

The invention is additionally directed to the embodiment of all of such above-indicated methods and uses, wherein the binding molecule capable of binding PD-1 or a natural ligand of PD-1 is administered Q2W starting on day 15.

The invention is additionally directed to the embodiment of all of such above-indicated methods and uses, wherein the binding molecule capable of binding PD-1 or a natural ligand of PD-1 is administered on day 1 of one or more of the F7DPs.

The invention is additionally directed to the embodiment of all of such above-indicated methods and uses, wherein binding molecule capable of binding PD-1 or a natural ligand of PD-1 comprises:
(a) a VH Domain and a VL Domain of pembrolizumab;
(b) a VH Domain and a VL Domain of nivolumab;
(c) a VH Domain and a VL Domain of cemiplimab;
(c) a VH domain and a VL domain of PD-1 mAb 1;
(d) a VH Domain and a VL Domain of atezolizumab;
(e) a VH Domain and a VL Domain of avelumab;
(f) a VH Domain and a VL Domain of durvalumab; or
(h) a VH domain and a VL domain of an antibody provided in Tables 3 or 4.

The invention is additionally directed to the embodiment of all of such above-indicated methods and uses, wherein the binding molecule capable of binding PD-1 or a natural ligand of PD-1 comprises the VH domain and a VL domain of PD-1 mAb 1.

The invention is additionally directed to the embodiment of all of such above-indicated methods and uses, wherein the binding molecule capable of binding PD-1 or a natural ligand of PD-1 is PD-1 mAb 1 IgG4.

The invention is additionally directed to the embodiment of all of such above-indicated methods and uses, wherein the binding molecule capable of binding PD-1 or a natural ligand of PD-1 is administered at a dose of about 1 mg/kg to about 3 mg/kg.

The invention is additionally directed to the embodiment of all of such above-indicated methods and uses further comprising administering one or more doses of said binding molecule capable of binding PD-1 or a natural ligand of PD-1 after a last dose of said CD123×CD3 binding molecule is administered.

The invention is additionally directed to the embodiment of all of such above-indicated methods and uses that further comprises administering a corticosteroid and/or an anti-IL-6 or anti-IL-6R antibody by intravenous infusion before, during and/or after the administration of the CD123×CD3 binding molecule. Particularly wherein the corticosteroid is selected from the group consisting of dexamethasone, methylprednisolone and hydrocortisone.

The invention is additionally directed to the embodiment of all of such above-indicated methods and uses, wherein dexamethasone is administered prophylactically. Particularly wherein dexamethasone is administered at a dosage of from about 10 mg to about 20 mg before administration of the CD123×CD3 binding molecule.

The invention is additionally directed to the embodiment of all of such above-indicated methods and uses, further comprises administering dexamethasone at a dosage of about 4 mg during and/or after administration of the CD123×CD3 binding molecule.

The invention is additionally directed to the embodiment of all of such above-indicated methods and uses, further comprises administering an anti-IL-6 or anti-IL-6R antibody after administration of the CD123×CD3 binding molecule. Particularly, wherein the anti-IL-6 or anti-IL-6R antibody is tocilizumab or siltuximab, and more particularly, wherein the anti-IL-6R antibody is tocilizumab administered at a dosage of about 4 mg/kg to about 8 mg/kg.

The invention is additionally directed to the embodiment of all of such above-indicated methods and uses, wherein the hematologic malignancy is selected from the group consisting of: acute myeloid leukemia (AML), chronic myelogenous leukemia (CML), including blastic crisis of CML and Abelson oncogene associated with CML (Bcr-ABL translocation), myelodysplastic syndrome (MDS), acute B lymphoblastic leukemia (B-ALL), acute T lymphoblastic leukemia (T-ALL), chronic lymphocytic leukemia (CLL), including Richter's syndrome or Richter's transformation of CLL, hairy cell leukemia (HCL), blastic plasmacytoid dendritic cell neoplasm (BPDCN), non-Hodgkin's lymphoma (NHL), including mantle cell lymphoma (MCL) and small lymphocytic lymphoma (SLL), Hodgkin's lymphoma, systemic mastocytosis, and Burkitt's lymphoma.

The invention is additionally directed to the embodiment of all of such above-indicated methods and uses, wherein the hematologic malignancy is acute myeloid leukemia.

The invention is additionally directed to the embodiment of all of such above-indicated methods and uses, wherein the hematologic neoplasm is myelodysplastic syndrome.

The invention is additionally directed to the embodiment of all of such above-indicated methods and uses, wherein the hematologic neoplasm is acute T lymphoblastic leukemia.

The invention is additionally directed to the embodiment of all of such above-indicated methods and uses, wherein the subject is a human.

### BRIEF DESCRIPTION OF THE DRAWINGS:

FIG. 1 illustrates the overall structure of the first and second polypeptide chains of two chain CD123×CD3 bispecific diabodies, such as DART-A.

FIGS. 2A-2D show the activity of the CD123×CD3 DART® molecules of the present invention on PMBCs of AML patients. Primary PBMCs (containing 82% blasts) were treated with DART-A, a FITC×CD3 control DART® molecule, or phosphate buffered saline (PBS) for 144 hours. The E:T cell ratio was approximately 1:300 as determined from blast and T cell percentages in PBMCs at the start of the study. FIG. 2A: absolute number of leukemic blast cells (CD45+/CD33+); FIG. 2B: absolute numbers of T cells (CD4+ and CD8+); FIG. 2C: T-cell activation (CD25 expression); FIG. 2D: cytokines measured in culture supernatants.

FIGS. 3A-3C show the analysis of PBMCs and blast cells from AML patients. FIG. 3A shows IFN-γ release following 48-hour incubation with 5, 50, or 500 pg/ml DART-A. FIG. 3B shows PD-1 upregulation on the cell surface of CD4⁺ and CD8⁺ T-cells following 48-hour incubation with 5, 50, or 500 pg/ml DART-A. FIG. 3C shows PD-L1 upregulation on the surface of AML blasts following 48 hour incubation with DART-A.

FIGS. 4A-4D show the cell surface expression and percent positivity of PD-1, respectively, on CD4⁺ T-cells (FIGS. 4A and 4B) or CD8⁺ T-cells (FIGS. 4C and 4D) obtained from a representative AML-PMBC sample, following incubation with DART-A (8.23, 24.69, 74.07, 222.22, 666.67, or 2000 pg/ml) with or without anti-PD-1 mAb (PD-1 mAb 1 IgG4; 10 μg/ml), or an isotype control antibody.

FIGS. 5A-5D show the in vitro release of GM-C SF (FIG. 5A), IFN-γ (FIG. 5B), IL-2 (FIG. 5C) and TNF-α (FIG. 5D) from a representative sample of AML-PBMC following incubation with DART-A (8.23, 24.69, 74.07, 222.22, 666.67, or 2000 pg/ml), with or without anti-PD-1 mAb (PD-1 mAb 1 IgG4; 10 μg/ml), or isotype control antibody, for 48 or 72 hours.

FIG. 6 shows the enhancement of killing of non-T-cells obtained from AML-PBMC following 72-hour treatment in vitro with DART-A (8.23, 24.69, 74.07, 222.22, 666.67, or 2000 pg/ml) with or without anti-PD-1 mAb (PD-1 mAb 1 IgG4; 10 μg/ml).

FIG. 7 shows an overview of the CRS grade exhibited during the first four weeks by participants administered DART-A using the one-step (LID-1 Schema) or two-step (LID-2 Schema) lead-in dosing strategy.

FIG. 8 shows the anti-leukemic activity of 14 patients treated at ≥500 ng/kg/day that received at least one cycle of treatment and had a post-treatment bone marrow biopsy (CR, Complete Response; CRm, molecular CR; CRi, Complete Response with incomplete hematological improvement; MLF, Morphologic Leukemia-free state; PR, Partial Response; SD/OB, Stable Disease/Other Anti-Leukemic Benefit; PD, Progressive Disease).

FIG. 9 shows the anti-leukemic activity of 34 response evaluable patients treated LID-2 with Continuous Dosage Schedule at 500 ng/kg/day target dose (Table 7). (CR, Complete Response; CRi, Complete Response with incomplete hematological improvement; MLF=Morphologic Leukemia-free state; PR, Partial Response; SD, Stable Disease; PD, Progressive Disease).

FIG. 10 shows the median duration of CRS events by Grade. CRS Grade 1 events: 1 day; CRS Grade 2 events: 2 days; and CRS Grade 3 events: 2.5 days.

FIG. 11 shows the number of CRS events per patient decreases over the first two weeks using a two-step Lead-in Dose (i.e., 30 ng/kg/day for 3 days followed by 100 ng/kg/day for 4 days) and first week of an additional 7-day treatment period (A7DP), during which the dose was maintained at a target dose of 500 ng/kg/day. Number of CRS events per patient (left axis) and the number of treated patients (right axis) is plotted over time for the first eight weeks of treatment.

FIGS. 12A-12B show an overview of the CRS grade exhibited by participants administered DART-A using the different lead-in dose strategies. FIG. 12A the mean IRR/CRS grade exhibited by 8 study participants administered DART-A using the multi-step LID-3 Schema (I7DP, target dose 500 ng/kg/day) followed by three weeks of continuous dosing at the target dose (A7DP 1-A7DP 3). FIG. 12B also plots the mean IRR/CRS grade exhibited using the multi-step, one-step (LID-1 Schema) and two-step (LID-2 Schema) lead-in dosing strategy.

FIGS. 13A-13B plot the average dose intensity of DART-A administered (solid lines) during cycle 1 using the different lead-in dose strategies. FIG. 13A plots the average dose intensity of DART-A administered to 30 patients using the 2-step LID-2 Schema. FIG. 13B plots the average dose intensity of DART-A administered to 30 patients using the multi-step LID-3 Schema and shows that on average 80.6% of the desired peak dose intensity (DI) of 500 ng/kg/day was achieved. The target maximum dose intensity of for each step is represented by the dashed line.

### DETAILED DESCRIPTION OF THE INVENTION

The present invention is directed to a dosing regimen for administering a CD123×CD3 bispecific diabody to patients with a hematologic malignancy such as acute myeloid leukemia (AML) or myelodysplastic syndrome (MDS). The present invention is also directed to a dosing regimen for administering a CD123×CD3 bispecific diabody in combination with a molecule capable of binding PD-1 or a natural ligand of PD-1 (a “PD-1 or PD-1 ligand binding molecule”) to patients with a hematologic malignancy such as acute myeloid leukemia (AML) or myelodysplastic syndrome (MDS). The invention particularly concerns the use of such regimens for the sequence-optimized CD123×CD3 bispecific diabody “DART-A,” that is capable of simultaneous binding to CD123 and CD3.

### I. The Polypeptide Chains of DART-A

DART-A is a sequence-optimized bispecific diabody capable of simultaneously and specifically binding to an epitope of CD123 and to an epitope of CD3 (a “CD123×CD3” bispecific diabody) (US Patent Publn. No. US 2016-0200827, in PCT Publn. WO 2015/026892, in Al-Hussaini, M. et al. (2016) “Targeting CD123 In Acute Myeloid Leukemia Using A T-Cell-Directed Dual Affinity Retargeting Platform,” Blood 127:122-131, in Vey, N. et al. (2017) “A Phase 1, First-in-Human Study of MGD006/580880 (CD123×CD3) in AML/MDS,” 2017 ASCO Annual Meeting, Jun. 2-6, 2017, Chicago, Ill.: Abstract TPS7070, each of which documents is herein incorporated by reference in its entirety). DART-A was found to exhibit enhanced functional activity relative to other non-sequence-optimized CD123×CD3 bispecific diabodies of similar composition, and is thus termed a “sequence-optimized” CD123×CD3 bispecific diabody.

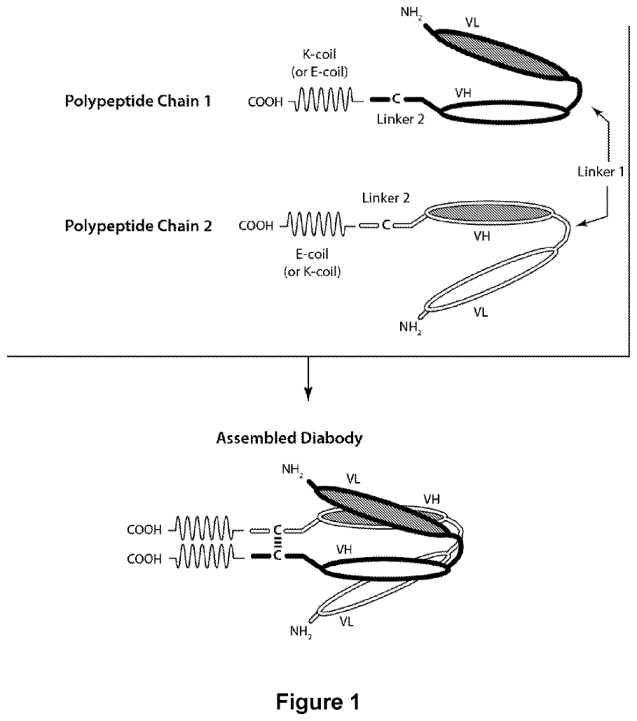

DART-A comprises a first polypeptide chain and a second polypeptide chain. The first polypeptide chain of the bispecific diabody will comprise, in the N-terminal to C-terminal direction, an N-terminus, a Light Chain Variable Domain (VL Domain) of a monoclonal antibody capable of binding to CD3 (VL_{CD3}), an intervening linker peptide (Linker 1), a Heavy Chain Variable Domain (VH Domain) of a monoclonal antibody capable of binding to CD123 (VH_{CD123}), and a C-terminus, and has the general structure provided in FIG. 1. A preferred sequence for such a VL_{CD3} Domain is SEQ ID NO:1:

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG

The Antigen Binding Domain of VL_{CD3} comprises:

CDR1 (SEQ ID NO: 2):
RSSTGAVTTSNYAN;
CDR2 (SEQ ID NO: 3):
GTNKRAP;
and
CDR3 (SEQ ID NO: 4):
ALWYSNLWV.

A preferred sequence for such Linker 1 is SEQ ID NO:5: GGGSGGGG. A preferred sequence for such a VH_{CD123} Domain is SEQ ID NO:6:

EVQLVQSGAE LKKPGASVKV SCKASGYTFT DYYMKWVRQA
PGQGLEWIGD IIPSNGATFY NQKFKGRVTI TVDKSTSTAY
MELSSLRSED TAVYYCARSH LLRASWFAYW GQGTLVTVSS

The Antigen Binding Domain of VH_{CD123} comprises:

CDR1 (SEQ ID NO: 7):
DYYMK;
CDR2 (SEQ ID NO: 8):
DIIPSNGATFYNQKFKG;
and
CDR3 (SEQ ID NO: 9):
SHLLRASWFAY.

The second polypeptide chain will comprise, in the N-terminal to C-terminal direction, an N-terminus, a VL domain of a monoclonal antibody capable of binding to CD123 (VL_{CD123}), an intervening linker peptide (e.g., Linker 1), a VH domain of a monoclonal antibody capable of binding to CD3 (VH_{CD3}), and a C-terminus. A preferred sequence for such a VL_{CD123} Domain is SEQ ID NO:10:

DFVMTQSPDS LAVSLGERVT MSCKSSQSLL NSGNQKNYLT
WYQQKPGQPP KLLIYWASTR ESGVPDRFSG SGSGTDFTLT
ISSLQAEDVA VYYCQNDYSY PYTFGQGTKL EIK

The Antigen Binding Domain of VL_{CD123} comprises:

CDR1 (SEQ ID NO: 11):
KSSQSLLNSGNQKNYLT;
CDR2 (SEQ ID NO: 12):
WASTRES;
and
CDR3 (SEQ ID NO: 13):
QNDYSYPYT.

A preferred sequence for such a VH_{CD3} Domain is SEQ ID NO:14:

EVQLVESGGG LVQPGGSLRL SCAASGFTFS TYAMNWVRQA
PGKGLEWVGR IRSKYNNYAT YYADSVKDRF TISRDDSKNS
LYLQMNSLKT EDTAVYYCVR HGNFGNSYVS WFAYWGQGTL
VTVSS

The Antigen Binding Domain of VH_{CD3} comprises:

CDR1
(SEQ ID NO: 15):
TYAMN;
CDR2
(SEQ ID NO: 16):
RIRSKYNNYATYYADSVKD;
and
CDR3
(SEQ ID NO: 17):
HGNFGNSYVSWFAY.

The sequence-optimized CD123×CD3 bispecific diabodies of the present invention are engineered so that such first and second polypeptides covalently bond to one another via cysteine residues along their length. Such cysteine residues may be introduced into the intervening linker (e.g., Linker 1) that separates the VL and VH domains of the polypeptides. Alternatively, and more preferably, a second peptide (Linker 2) is introduced into each polypeptide chain, for example, at a position N-terminal to the VL domain or C-terminal to the VH domain of such polypeptide chain. A preferred sequence for such Linker 2 is SEQ ID NO:18: GGCGGG.

The formation of heterodimers can be driven by further engineering such polypeptide chains to contain polypeptide coils of opposing charge. Thus, in a preferred embodiment, one of the polypeptide chains will be engineered to contain an “E-coil” domain (SEQ ID NO:19: [symbol] VAAL [symbol] K [symbol] VAAL [symbol] K [symbol] VAAL [symbol] K [symbol] VAAL [symbol] K) whose residues will form a negative charge at pH 7, while the other of the two polypeptide chains will be engineered to contain an “K-coil” domain (SEQ ID NO:20: [symbol] VAAL [symbol] E [symbol] VAAL [symbol] E [symbol] VAAL [symbol] E [symbol] VAAL [symbol] E) whose residues will form a positive charge at pH 7. The presence of such charged domains promotes association between the first and second polypeptides, and thus fosters heterodimerization.

It is immaterial which coil is provided to the first or second polypeptide chains. However, a preferred sequence-optimized CD123×CD3 bispecific diabody of the present invention (“DART-A”) has a first polypeptide chain having the sequence (SEQ ID NO:21):

QAVVTQEPSL TVSPGGTVTL TCRSSTGAVT
TSNYANWVQQ KPGQAPRGLI GGTNKRAPWT
PARFSGSLLG GKAALTITGA QAEDEADYYC
ALWYSNLWVF GGGTKLTVLG GGGSGGGGEV
QLVQSGAELK KPGASVKVSC KASGYTFTDY
YMKWVRQAPG QGLEWIGDII PSNGATFYNQ
KFKGRVTITV DKSTSTAYME LSSLRSEDTA
VYYCARSHLL RASWFAYWGQ GTLVTVSSGG
CGGGEVAALE KEVAALEKEV AALEKEVAAL
EK

DART-A Chain 1 is composed of: SEQ ID NO:1-SEQ ID NO:5-SEQ ID NO:6-SEQ ID NO:18-SEQ ID NO:19. A DART-A Chain 1 encoding polynucleotide is SEQ ID NO:22:

caggctgtgg tgactcagga gccttcactg
accgtgtccc caggcggaac tgtgaccctg
acatgcagat ccagcacagg cgcagtgacc
acatctaact acgccaattg ggtgcagcag
aagccaggac aggcaccaag gggcctgatc
gggggtacaa acaaaagggc tccctggacc
cctgcacggt tttctggaag tctgctgggc
ggaaaggccg ctctgactat taccggggca
caggccgagg acgaagccga ttactattgt
gctctgtggt atagcaatct gtgggtgttc
gggggtggca caaaactgac tgtgctggga
gggggtggat ccggcggcgg aggcgaggtg
cagctggtgc agtccggggc tgagctgaag
aaacccggag cttccgtgaa ggtgtcttgc
aaagccagtg gctacacctt cacagactac
tatatgaagt gggtcaggca ggctccagga
cagggactgg aatggatcgg cgatatcatt
ccttccaacg gggccacttt ctacaatcag
aagtttaaag gcagggtgac tattaccgtg
gacaaatcaa caagcactgc ttatatggag
ctgagctccc tgcgctctga agatacagcc
gtgtactatt gtgctcggtc acacctgctg
agagccagct ggtttgctta ttggggacag
ggcaccctgg tgacagtgtc ttccggagga
tgtggcggtg gagaagtggc cgcactggag
aaagaggttg ctgctttgga gaaggaggtc
gctgcacttg aaaaggaggt cgcagccctg
gagaaa

The second polypeptide chain of DART-A has the sequence (SEQ ID NO:23):

DFVMTQSPDS LAVSLGERVT MSCKSSQSLL
NSGNQKNYLT WYQQKPGQPP KLLIYWASTR
ESGVPDRFSG SGSGTDFTLT ISSLQAEDVA
VYYCQNDYSY PYTFGQGTKL EIKGGGSGGG
GEVQLVESGG GLVQPGGSLR LSCAASGFTF
STYAMNWVRQ APGKGLEWVG RIRSKYNNYA
TYYADSVKDR FTISRDDSKN SLYLQMNSLK
TEDTAVYYCV RHGNFGNSYV SWFAYWGQGT
LVTVSSGGCG GGKVAALKEK VAALKEKVAA
LKEKVAALKE

DART-A Chain 2 is composed of: SEQ ID NO:10-SEQ ID NO:5-SEQ ID NO:14-SEQ ID NO:18-SEQ ID NO:20. A DART-A Chain 2 encoding polynucleotide is SEQ ID NO:24:

gacttcgtga tgacacagtc tcctgatagt
ctggccgtga gtctggggga gcgggtgact
atgtcttgca agagctccca gtcactgctg
aacagcggaa atcagaaaaa ctatctgacc
tggtaccagc agaagccagg ccagccccct
aaactgctga tctattgggc ttccaccagg
gaatctggcg tgcccgacag attcagcggc
agcggcagcg gcacagattt taccctgaca
atttctagtc tgcaggccga ggacgtggct
gtgtactatt gtcagaatga ttacagctat
ccctacactt tcggccaggg gaccaagctg
gaaattaaag gaggcggatc cggcggcgga
ggcgaggtgc agctggtgga gtctggggga
ggcttggtcc agcctggagg gtccctgaga
ctctcctgtg cagcctctgg attcaccttc
agcacatacg ctatgaattg ggtccgccag
gctccaggga aggggctgga gtgggttgga
aggatcaggt ccaagtacaa caattatgca
acctactatg ccgactctgt gaaggataga
ttcaccatct caagagatga ttcaaagaac
tcactgtatc tgcaaatgaa cagcctgaaa
accgaggaca cggccgtgta ttactgtgtg
agacacggta acttcggcaa ttcttacgtg
tcttggtttg cttattgggg acaggggaca
ctggtgactg tgtcttccgg aggatgtggc
ggtggaaaag tggccgcact gaaggagaaa
gttgctgctt tgaaagagaa ggtcgccgca
cttaaggaaa aggtcgcagc cctgaaagag

### II. The Properties of DART-A

DART-A was found to have the ability to simultaneously bind CD123 and CD3 as arrayed by human and cynomolgus monkey cells. Provision of DART-A was found to cause T cell activation, to mediate blast reduction, to drive T cell expansion, to induce T cell activation and to cause the redirected killing of target cancer cells (Table 1).

TABLE 1
Equilibrium Dissociation Constants (K_{D}) for the Binding of
DART-A to Human and Cynomolgus Monkey CD3 and CD123

| | kₐ (± SD) | k_{d} (± SD) | K_{D} (± SD) |
|---|---|---|---|
| Antigens | (M⁻¹s⁻¹) | (s⁻¹) | (nM) |
| Human CD3ε/δ | 5.7 (± 0.6) × 10⁵ | 5.0 (± 0.9) × 10⁻³ | 9.0 ± 2.3 |
| Cynomolgus CD3ε/δ | 5.5 (± 0.5) × 10⁵ | 5.0 (± 0.9) × 10⁻³ | 9.2 ± 2.3 |
| Human CD123-His | 1.6 (± 0.4) × 10⁶ | 1.9 (± 0.4) × 10⁻⁴ | 0.13 ± 0.01 |
| Cynomolgus | 1.5 (± 0.3) × 10⁶ | 4.0 (± 0.7) × 10⁻⁴ | 0.27 ± 0.02 |
| CD123-His | | | |

More particularly, DART-A was found to exhibit a potent redirected killing ability with concentrations required to achieve 50% of maximal activity (EC50s) in sub-ng/mL range, regardless of CD3 epitope binding specificity in target cell lines with high CD123 expression (Kasumi-3 (EC50=0.01 ng/mL)) medium CD123-expression (Molm13 (EC50=0.18 ng/mL) and THP-1 (EC50=0.24 ng/mL)) and medium low or low CD123 expression (TF-1 (EC50=0.46 ng/mL) and RS4-11 (EC50=0.5 ng/mL)). Similarly, DART-A-redirected killing was also observed with multiple target cell lines with T cells from different donors and no redirected killing activity was observed in cell lines that do not express CD123. Results are summarized in Table 2.

TABLE 2

| | | EC50 of Sequence- | |
|---|---|---|---|
| | CD123 surface | optimized CD123 × | |
| | expression | CD3 bispecific | |
| | (antibody | diabodies (ng/mL) | |
| Target cell line | binding sites) | E:T = 10:1 | Max % killing |
| Kasumi-3 | 118620 | 0.01 | 94 |
| Molm13 | 27311 | 0.18 | 43 |
| THP-1 | 58316 | 0.24 | 40 |
| TF-1 | 14163 | 0.46 | 46 |
| RS4-11 | 957 | 0.5 | 60 |
| A498 | Negative | No activity | No activity |
| HT29 | Negative | No activity | No activity |

Additionally, when human T cells and tumor cells (Molm13 or RS4-11) were combined and injected subcutaneously into NOD/SCID gamma (NSG) knockout mice, the MOLM13 tumors was significantly inhibited at the 0.16, 0.5, 0.2, 0.1, 0.02, and 0.004 mg/kg dose levels. A dose of 0.004 mg/kg and higher was active in the MOLM13 model. The lower DART-A doses associated with the inhibition of tumor growth in the MOLM13 model compared with the RS4-11 model are consistent with the in vitro data demonstrating that MOLM13 cells have a higher level of CD123 expression than RS4-11 cells, which correlated with increased sensitivity to DART-A mediated cytotoxicity in vitro in MOLM13 cells.

DART-A was found to be active against primary AML specimens (bone marrow mononucleocytes (BMNC) and peripheral blood mononucleocytes (PBMC)) from AML patients. Incubation of primary AML bone marrow samples with DART-A resulted in depletion of the leukemic cell population over time, accompanied by a concomitant expansion of the residual T cells (both CD4 and CD8) and the induction of T cell activation markers (CD25 and Ki-67). Upregulation of granzyme B and perforin levels in both CD8 and CD4 T cells was observed. Incubation of primary ALL bone marrow samples with DART-A resulted in depletion of the leukemic cell population over time compared to untreated control or Control DART. When the T cells were counted (CD8 and CD4 staining) and activation (CD25 staining) were assayed, the T cells expanded and were activated in the DART-A sample compared to untreated or Control DART samples. DART-A was also found to be capable of mediating the depletion of pDCs cells in both human and cynomolgus monkey PBMCs, with cynomolgus monkey pDCs being depleted as early as 4 days post infusion with as little as 10 ng/kg DART-A. No elevation in the levels of cytokines interferon-gamma, TNFα, IL-6, IL-5, IL-4 and IL-2 were observed in DART-A-treated animals. These data indicate that DART-A-mediated target cell killing was mediated through a granzyme B and perforin pathway.

No activity was observed against CD123-negative targets (U937 cells) or with Control DART, indicating that the observed T cell activation was strictly dependent upon target cell engagement and that monovalent engagement of CD3 by DART-A was insufficient to trigger T cell activation.

In sum, DART-A is an antibody-based molecule engaging the CD3ε subunit of the TCR to redirect T lymphocytes against cells expressing CD123, an antigen up-regulated in several hematologic malignancies. DART-A binds to both human and cynomolgus monkey's antigens with similar affinities and redirects T cells from both species to kill CD123+ cells. Monkeys infused 4 or 7 days a week with weekly escalating doses of DART-A showed depletion of circulating CD123+ cells 72 h after treatment initiation that persisted throughout the 4 weeks of treatment, irrespective of dosing schedules. A decrease in circulating T cells also occurred, but recovered to baseline before the subsequent infusion in monkeys on the 4-day dose schedule, consistent with DART-A-mediated mobilization. DART-A administration increased circulating PD1+, but not TIM-3+, T cells; furthermore, ex vivo analysis of T cells from treated monkeys exhibited unaltered redirected target cell lysis, indicating no exhaustion. Toxicity was limited to a minimal transient release of cytokines following the DART-A first infusion, but not after subsequent administrations even when the dose was escalated, and a minimal reversible decrease in red cell mass with concomitant reduction in CD123+ bone marrow progenitors.

### III. Exemplary Molecules Capable Of Binding PD-1 or a Natural Ligand of PD-1

A. PD-1 Binding Molecules

Antibodies that are immunospecific for PD-1 and other molecules capable of binding PD-1 are known and may be employed or adapted to serve as a molecule (e.g., a multispecific binding molecule (e.g., a diabody, a bispecific antibody, a trivalent binding molecule, etc.), an antigen binding fragment of an antibody (e.g., an scFv, a Fab, a F(ab)2, etc.), an scFv fusion, etc.) capable of binding PD-1 in accordance with the present invention (see, e.g., the patent publications presented in Table 3 below). Preferred molecules capable of binding PD-1 will exhibit the ability to bind a continuous or discontinuous (e.g., conformational) portion (epitope) of human PD-1 (CD279) and will preferably also exhibit the ability to bind PD-1 molecules of one or more non-human species, in particular, primate species (and especially a primate species, such as cynomolgus monkey). In certain embodiments, molecules capable of binding PD-1 will exhibit the ability antagonize PD-1/PD-L1 interactions, for example by blocking binding between PD-1 and a natural ligand of PD-1. Additional desired antibodies may be made by isolating antibody-secreting hybridomas elicited using PD-1 or a peptide fragment thereof. A representative human PD-1 polypeptide (NCBI Sequence NP_005009.2; including a 20 amino acid residue signal sequence, shown underlined) and the 268 amino acid residue mature protein) has the amino acid sequence (SEQ ID NO:25):

[symbol]

[symbol]

PGWFLDSPDR
PWNPPTFSPA LLVVTEGDNA TFTCSFSNTS
ESFVLNWYRM SPSNQTDKLA AFPEDRSQPG
QDCRFRVTQL PNGRDFHMSV VRARRNDSGT
YLCGAISLAP KAQIKESLRA ELRVTERRAE
VPTAHPSPSP RPAGQFQTLV VGVVGGLLGS
LVLLVWVLAV ICSRAARGTI GARRTGQPLK
EDPSAVPVFS VDYGELDFQW REKTPEPPVP
CVPEQTEYAT IVFPSGMGTS SPARRGSADG
PRSAQPLRPE DGHCSWPL

Anti-PD-1 antibodies may be obtained using proteins having all or a portion of the above-provided PD-1 amino acid sequence as an immunogen. Alternatively, anti-PD-1 antibodies useful in the generation of molecules capable of PD-1 may possess the VL and/or VH Domains of the anti-human PD-1 described below or of an anti-PD-1 antibody listed in Table 3; and more preferably possess 1, 2 or all 3 of the CDR_{LS} of the VL Domain and/or 1, 2 or all 3 of the CDR_{HS} of the VH Domain of such anti-PD-1 antibodies.

One such exemplary humanized anti-PD-1 antibody is designated herein as “PD-1 mAb 1.” The amino acid sequence of the VH Domain of PD-1 mAb 1 (SEQ ID NO:26) is shown below (CDR_{H} residues are shown underlined):

QVQLVQSGAE VKKPGASVKV SCKASGYSFT [symbol]
WVRQA PGQGLEWIG [symbol] [symbol] [symbol]
RVTI TVDKSTSTAY MELSSLRSED
TAVYYCAR [symbol] [symbol] WG QGTLVTVSS

The amino acid sequence of the VL Domain of PD-1 mAb 1 (SEQ ID NO:27) is shown below (CDR_{H} residues are shown underlined):

EIVLTQSPAT LSLSPGERAT LSC [symbol] [symbol]
QQKPGQPPKL LIH [symbol]
GVPSRFSGSG SGTDFTLTIS SLEPEDFAVY
FC [symbol] [symbol] FGGGTKVEI K

Alternative anti-PD-1 antibodies and PD-1 binding molecules useful in the generation of molecules capable of binding PD-1 possess the VL and/or VH Domains of the anti-human PD-1 antibody nivolumab (CAS Reg. No.: 946414-94-4, also known as 5C4, BMS-936558, ONO-4538, MDX-1106, and marketed as OPDIVO® by Bristol-Myers Squibb); pembrolizumab (formerly known as lambrolizumab), CAS Reg. No.: 1374853-91-4, also known as MK-3475, SCH-900475, and marketed as KEYTRUDA® by Merck); cemiplimab (CAS Reg. No.: 1801342-60-8, also known as REGN-2810, SAR-439684, and marketed as LIBTAYO®), EH12.2H7 (Dana Farber), or any of the anti-PD-1 antibodies in Table 3; and more preferably possess 1, 2 or all 3 of the CDR_{LS} of the VL Domain and/or 1, 2 or all 3 of the CDR_{HS} of the VH Domain of such anti-PD-1 antibodies. The amino acid sequences of the complete Heavy and Light Chains of nivolumab (WHO Drug Information, 2013, Recommended INN: List 69, 27(1):68-69), pembrolizumab (WHO Drug Information, 2014, Recommended INN: List 75, 28(3):407), and cemiplimab (WHO Drug Information 2018, Proposed INN: List 119) are known in the art. Additional anti-PD-1 antibodies possessing unique binding characteristics useful in the methods and compositions of the instant inventions have recently been identified (see, PCT Publication No. WO 2017/019846 and Table 3).

TABLE 3
Additional Molecules that Bind PD-1

| Designation | Reference |
|---|---|
| PD1-17; PD1-28; PD1-33; PD1-35; and PD1-F2 | US 7,488,802 |
| 17D8; 2D3; 4H1; 5C4; 4A11; 7D3; and 5F4 | US 8,008,449 |
| hPD-1.08A; hPD-1.09A; 109A; K09A; 409A; | US 8,354,509 |
| h409A11; h409A16; h409A17; Codon | |
| optimized 109A; and Codon optimized 409A | |
| 1B8; 20B3.1; 7G3; 3H4; 2.3A9; 1G7; | US 8,168,757 |
| 1.8A10; 28.11; 6D10 | |
| 1E3; 1E8; and 1H3 | US 2014/0044738 |
| 9A2; 10B11; 6E9; APE1922; APE1923; | US 9,815,897 |
| APE1924; APE1950; APE1963; and APE2058 | |
| EH12.2H7 | US 9,102727 |
| GA1; GA2; GB1; GB6; GH1; A2; C7; H7; | US 2014/0356363 |
| SH-A4; SH-A9; RG1H10; RG1H11; RG2H7; | |
| RG2H10; RG3E12; RG4A6; RG5D9; | |
| RG1H10-H2A-22-1S; RG1H10-H2A-27-2S; | |
| RG1H10-3C; RG1H10-16C; RG1H10-17C; | |
| RG1H10-19C; RG1H10-21C; and RG1H10-23C2 | |
| H1M7789N; H1M7799N; H1M7800N; | US 2015/0203579 |
| H2M7780N; H2M7788N; H2M7790N; | |
| H2M7791N; H2M7794N; H2M7795N; | |
| H2M7796N; H2M7798N; H4H9019P; | |
| H4H7798N; H4xH9034P2; H4xH9035P2; | |
| H4xH9037P2; H4xH9045P2; H4xH9048P2; | |
| H4H9057P2; H4H9068P2; H4xH9119P2; | |
| H4xH9120P2; H4Xh9128p2; H4Xh9135p2; | |
| H4Xh9145p2; H4Xh8992p; H4Xh8999p; | |
| and H4Xh9008p; | |
| mAb1; mAb2; mAb3; mAb4; mAb7; | US 2016/0159905 |
| mAb8; mAb9; mAb10; mAb11; mAb12; | |
| mAb13; mAb14; mAb15; and mAb16 | |
| 246A10; 244C8; 413D2; 393C5; 388D4; | US 2016/0319019 |
| 413E1; 244C8-1; 244C8-2; 244C8-3; | |
| 388D4-1; 388D4-2; and 388D4-3 | |
| Mu317; mu326; 317-4B6; 326-4A3; | US 8,735,553 |
| 317-4B2; 317-4B5; 317-1; 326-3B1; | |
| 326-3G1; 326-1; 317-3A1; 317-3C1; | |
| 317-3E1; 317-3G1; 317-3H1; 317-3I1; | |
| 317-4B1; 317-4B3; 317-4B4; 317-4A2; | |
| 326-3A1; 326-3C1; 326-3D1; 326-3E1; | |
| 326-3F1; 326-3B N55D; 326-4A1; | |
| 326-4A2BGB-A317 | |
| 22A5; 6El; lODl, 4C1; 7D3; 13Fl; 14A6; | US 2017/267762 |
| 15H5; 5A8; 7A4; and humanized | |
| versions of the same | |
| 1E9; hlE9-1; hlE9-2; hlE9-4; hlE9-5; | US 2018/142022 |
| 4B10; h4B10-1; h4B10-2; h4B10-3; | |
| 1B10; 10B4; A09; C07; F09; G08; | |
| G10; H08; H09; and 1353-G10 | |
| M136-M13-MHC723; m136-M14- | US 2017/0044259 |
| MHC724; m136-M19-MHC725; | |
| m245-M3-MHC728; m245-M5-MHC729; | |
| A1.0; A1.6; Ba2; Bb2/C1.1; and D4 | |
| PD-1 mAb 1; PD-1 mAb 2; PD-1 mAb 3; | US 2017/019846 |
| PD-1 mAb 4; PD-1 mAb 5; PD-1 mAb 6; | |
| PD-1 mAb 7; PD-1 mAb 8; PD-1 | |
| mAb 9; PD-1 mAb 10; PD-1 mAb 11; | |
| PD-1 mAb 12; PD-1 mAb 13; PD-1 | |
| mAb 14; PD-1 mAb 15; and humanized | |
| versions of the same: hPD-1 mAb 2; | |
| hPD-1 mAb 7; hPD-1 mAb 9; hPD-1 mAb 15; | |
| PD1B11; PD1B70; PD1B71; PD1B114 and | US 20017/079112 |
| affinity-matured variants there of: | |
| PD1B149; PD1B160; PD1B162; | |
| PD1B164; PD1B183; PD1B184; | |
| PD1B185; PD1B187; PD1B192; | |
| PD1B175; PD1B177; PD1B194; | |
| PD1B195; PD1B196; PD1B197; | |
| PD1B198; PD1B199; PD1B200; PD1B201 | |
| BAP049-hum01; BAP049-hum02; | US 2018/0371093 |
| BAP049-hum03; BAP049-hum04; | |
| BAP049-hum05; BAP049-hum06; | |
| BAP049-hum07; BAP049-hum08; | |
| BAP049-hum09; BAP049-huml0; | |
| BAP049-huml1; BAP049-huml2; | |
| BAP049-huml3; BAP049-huml4; | |
| BAP049-huml5; BAP049-huml6; | |
| BAP049-Clone-A; BAP049-Clone-B; | |
| BAP049-Clone-C; BAP049-Clone-D; | |
| or BAP049-Clone-E; PDR-001 | |
| AGEN-2034; AGEN-2034w; | US 2017/081409 |
| AGEN2033w; AGEN2046w; | |
| AGEN2047w; AGEN2001w; | |
| AGEN2002w; EPl l_pll_B03; | |
| EPl l_pll_B05; EPl l_pll_C02; EPl l_pll_C03 | |
| m136-M13− MHC723; m136-M19− | US 2017/044259 |
| MHC725; m245-M3− MHC728; | |
| m245-M5− MHC729; m136-M14− MHC724; | |
| and humanized variants PD-1 A; | |
| PD-1 Ab; PD-1 Ae; PD-1 Af; PD-1 Ba; | |
| PD-1 Bb; PD-1 C; PD-1 Ca; PD-1 D; PD-1 | |
| 1.0; PD-1 1.1; PD-1 1.2; PD-1 1.4; | |
| PD-1 1.5; PD-1 1.6; PD-1 1.7; PD-1 | |
| 1.9; PD-1 1.10; PD-1 2; PD-1 4; CX188 | |
| 244C8; 388D4; 413E1; 246A10; 413D2; | US 10,239,942 |
| and humanized variants D4-HC3+LC1; | |
| D4-HC1+LC3; D4-HC3+LC3; C8- | |
| HC1+LC1; C8-HC1+LC3; C8-HC2+LC1 | |
| PRS-332; VH selected from SEQ ID NOs: | US 2019/010231 |
| 59-84 and 112-117; and VL selected | |
| from SEQ ID NOs: 85-111 and 118-123 | |
| H005-1 | US 2016/376367 |
| BA08-1 | US 2017/210806 |
| R3A1; R3A2; R4B3; R3B7; R3D6; | US 2018/244779 |
| A2_#1; A2_#2 | |
| BY18.1 | WO 2016/180034 |
| Antibody A, Antibody B, Antibody C, | US 2017/0044260 |
| Antibody D, Antibody E, Antibody F, | |
| Antibody G, Antibody H, Antibody I; 11430 | |
| SHB-128; SHB-152; SHB-168; SHB-617; | US 2018/346569 |
| and humanized variant SSI-361 | |
| E8-3; C2-3; E1-3; F3-3; H8-3; C10-2; | US 9,982,052 |
| G2-1; G3-2; H2-1; H4-2; C8-1; | |
| G10-2; 135C12; 136B4; 139D6; 136E10; | |
| 122F10; 139D6; 137F2 | |
| AB12M3; AB12M4; AB12M5; AB12M6; | US 2018/113258 |
| AB12M7; AB12M8; AB12M9 | |
| 1.7.3 hAb; 1.49.9 hAb; 1.103.11 hAb; | US 2017/024515; |
| 1.139.15 hAb; 1.153.7 hAb | US 2017/025051 |
| 949 and humanized variants including | US 9,102,728 |
| 949 VK1 gL9 gH8b | |
| 948 and humanized variants | US 8,993,731 |
| STM-432 | US 2019/077866 |

B. PD-1 Ligand Binding Molecules

Antibodies that are immunospecific for a natural ligand of PD-1 (e.g., B7-H1 (PD-L1, CD274), B7-DC (PD-L2, CD273)) and molecules capable of binding a natural ligand of PD-1 are known and may be employed or adapted to serve as a molecule (e.g., a multispecific binding molecule (e.g., a diabody, a bispecific antibody, a trivalent binding molecule, etc.), an antigen binding fragment of an antibody (e.g., an scFv, a Fab, a F(ab)2, etc.), an scFv-Fc fusion, etc.) capable of binding a natural ligand of PD-1 in accordance with the present invention (see, e.g., the patent publications presented in Table 4 below). Preferred molecules capable of binding a natural ligand of PD-1 will exhibit the ability to bind a continuous or discontinuous (e.g., conformational) portion (epitope) of human B7-H1 and/or B7-DC and will preferably also exhibit the ability to bind B7-H1 and/or B7-DC molecules of one or more non-human species, in particular, primate species (and especially a primate species, such as cynomolgus monkey). In certain embodiments, molecules capable of binding a natural ligand of PD-1 will exhibit the ability antagonize PD-1/PD-L1 interactions, for example by blocking binding between PD-1 and a natural ligand of PD-1. Additional desired antibodies may be made by isolating antibody-secreting hybridomas elicited using B7-H1, B7-DC or a peptide fragment thereof.

A representative human B7-H1 (PD-L1) polypeptide (NCBI Sequence NP_001254635.1, including a predicted 18 amino acid signal sequence) has the amino acid sequence (SEQ ID NO:28):

MRIFAVFIFM TYWHLLNAPY NKINQRILVV
DPVTSEHELT CQAEGYPKAE VIWTSSDHQV
LSGKTTTTNS KREEKLFNVT STLRINTTTN
EIFYCTFRRL DPEENHTAEL VIPELPLAHP
PNERTHLVIL GAILLCLGVA LTFIFRLRKG
RMMDVKKCGI QDTNSKKQSD THLEET

A representative human B7-DC (PD-L2) polypeptide (NCBI Sequence NP_079515.2; including a predicted 18 amino acid signal sequence) has the amino acid sequence (SEQ ID NO:29):

MIFLLLMLSL ELQLHQIAAL FTVTVPKELY
IIEHGSNVTL ECNFDTGSHV NLGAITASLQ
KVENDTSPHR ERATLLEEQL PLGKASFHIP
QVQVRDEGQY QCIIIYGVAW DYKYLTLKVK
ASYRKINTHI LKVPETDEVE LTCQATGYPL
AEVSWPNVSV PANTSHSRTP EGLYQVTSVL
RLKPPPGRNF SCVFWNTHVR ELTLASIDLQ
SQMEPRTHPT WLLHIFIPFC IIAFIFIATV
IALRKQLCQK LYSSKDTTKR PVTTTKREVN
SAI

In particular, anti-B7-H1 antibodies may be obtained using proteins having a portion or all of the above-provided B7-H1 amino acid sequence as an immunogen. Alternatively, anti-B7-H1 1 antibodies useful in the generation of molecules capable of B7-H1 may possess the VL and/or VH Domains of the anti-human B7-H1 described below or of an anti-B7-H1 antibody listed in Table 4; and more preferably possess 1, 2 or all 3 of the CDR_{LS} of the VL Domain and/or 1, 2 or all 3 of the CDR_{HS} of the VH Domain of such anti-B7-H1 antibodies.

Exemplary anti-B7-H1 antibodies useful in the generation of molecules capable of binding a natural ligand of PD-1 may possess the VL and/or VH Domains of the anti-human B7-H1 antibody atezolizumab (CAS Reg No. 1380723-44-3, also known as MPDL3280A, and marketed as TECENTRIQ®), durvalumab (CAS Reg No. 1428935-60-7, also known as MEDI-4736, and marketed as IMFINZI®), avelumab, MDX1105 (CAS Reg No. 1537032-82-8, also known as BMS-936559, 5H1, and marketed as BAVENCIO®), or of any of the anti-B7-H1 antibodies and binding molecules listed in Table 4; and more preferably possess 1, 2 or all 3 of the CDR_{LS} of the VL Domain and/or 1, 2 or all 3 of the CDR_{HS} of the VH Domain of such anti-B7-H1 antibodies. The amino acid sequences of the complete heavy and Light Chains of atezolizumab (WHO Drug Information, 2015, Recommended INN: List 74, 29(3):387), durvalumab (WHO Drug Information, 2015, Recommended INN: List 74, 29(3):393-394) and avelumab (WHO Drug Information, 2016, Recommended INN: List 74, 30(1):100-101) are known in the art.

TABLE 4
Additional Molecules That Bind A Natural Ligand of PD-1

| Designation | Reference |
|---|---|
| A09-188-1, and affinity matured and | US 9,624298 |
| optimized variants: A09-204-1, A09-211-1, | |
| A09-212-1, A09-213-1, A09-214-1, | |
| A09-215-1, A09-216-1, A09-219-1, | |
| A09-220-1, A09-221-1, A09-222-1, A09-223-1, | |
| A09-202-1, A09-248-2, A09-239-2, A09-240-2, | |
| A09-241-2, A09-242-2, A09-243-2, A09-244-2, | |
| A09-245-2, A09-246-2, A09-247-2 | |
| YW243.55.S70; 243.55.H1; 243.55.H12; | US 8,217,149 |
| 243.55.H37; 243.55.H70; 243.55.H89; 243.55.S1; | |
| 243.55.5; 243.55.8; 243.55.30; 243.55.34; | |
| 243.55.S37; 243.55.49; 243.55.51; | |
| 243.55.62; 243.55.84 | |
| 2.9D10, 2.7A4, 2.14H9, 3.15G8, 2.20A8, | US 8,779108B2 |
| 3.18G1, 2.7A4OPT, or 2.14H9OPT | |
| 1B9.2E11.2, 4H1.G10.15, 1A8, 1E4, | US 2015/0197571 |
| 8G2, 1D11, 3A2, 3B11, 3F4, 3H6, 4C1, | |
| 4E1, 5A6, 9C12, 1B4, 1B11, 1F6, 1H8, | |
| 1H12, 2D5, 2H11, 3D12, 4C8, 4C9, | |
| 5E10, 5H4, 5H5, 8A1, 9G9, 10A7, and 10H6 | |
| 1D05, 84G09, 411B08, 411C04, 411D07, | US 9,617,338 |
| 386H03, 386A03, 385F01, 413D08, | |
| 413G05, 413F09, 414B06 | |
| 3G10, 12A4, 10A5, 5F8, 10H10, 1B12, | US 9,273,135 |
| 7H1, 11E6, 12B7, and 13G4 | |
| A1, C2, C4, H12, and H12-GL | US 2017/0319690 |
| Ab-14, Ab-16, Ab-22, Ab-30, Ab-31, | US 9,828434 |
| Ab-32, Ab-38, Ab-42, Ab-46, Ab-50, | |
| Ab-52, Ab-55, Ab-56, and Ab-65. | |
| R2κA3, R2κA4, R2κA6, R2κF4, | US 2016/340429 |
| R2κH5, R2κH6, R2κH3, sR3κA8, | |
| sR3κA9, sR3κB2, sR3κB5, tccR3KA8, | |
| tccR3KAl1, tccR3KB7, tccR3KD9, | |
| tccKF10, tctR3KA4, tctR3KF8, | |
| R2λA7, R2λB12, R2λ12, sR3λD7, | |
| sR3λE1, tccAF8, tccAD7, | |
| tctR3λH4, KD-033, and others | |
| H2M8306N, H2M8307N, H2M8309N, | US 9,938345 |
| H2M8310N, H2M8312N, H2M8314N, | |
| H2M8316N, H2M8317N, H2M8321N, | |
| H2M8323N, H2M8718N, H2M8718N2, and | |
| H2M8719N, H1H9323P, H1 H9327P, | |
| H1 H9329P, H1H9336P, H1H9344P2, | |
| 1H9345P2, H1H9351P2, H1H9354P2, | |
| H1 H9364P2, H1H9373P2, H1H9382P2, | |
| H1H9387P2, and H1H9396P2 | |
| clone 8, clone 12, clone 16, clone 18, | US 2016/0311903 |
| clone 60; and optimized variants thereof | |
| including: cl; dl; g7; h9; b10; E10; A05; C05; | |
| C10; D08; G09; G10; G12; Ell; D01; H06; | |
| C5H9; C5B10; C5E10; G12H9; | |
| G12B10; G12E10; | |
| BAP058 and humanized variants thereof | US 9,988,452 |
| including: BAP058-hum01, BAP058- | |
| hum02, BAP058-hum03, BAP058-hum04, | |
| BAP058-hum05, BAP058-hum06, BAP058-hum07, | |
| BAP058-hum08, BAP058-hum09, BAP058-hum10, | |
| BAP058-hum11, BAP058-hum12, BAP058-hum13, | |
| BAP058-hum14, BAP058-hum15, BAP058-hum16, | |
| and BAP058-hum17; FAZ-053 | |
| Mu333, Mu277, and humanized variants | US 2018/215825 |
| thereof including: hu333-2B, hu333- | |
| 3A2, hu333-3C2 and hu333-3H2 | |
| 332M1 and humanized variants there of | US 2018/346571 |
| including: 332M7, 332M72, and 332M8 | |
| PDL1.1; PDL1.2 | US 8,741295 |
| 13C5, 5G9, 5G11, 8C6, 7B4, 4D1, | US 2017/0204184 |
| 4A8, 8H4, 8H3, 15F1; and humanized | |
| variants thereof including hu5G11; hu13C5; | |
| PDL1-56 dAb; Hu56V1; Hu56V2; | US 2018/0291103 |
| Hu56V3; Hu56V4; Hu56V5; and KN035 | |
| 1.4.1, 1.14.4, 1.20.15 and 1.46.11 | WO 2017/020858 |
| 92; 24D5; 29H1; 9_2-1; 9_2-2; 9_2-3; | US 2018/0334504 |
| 9_2-4; 9_2-5; 9_2-6; 9_2-7; 9_2-8; 9_2-9; | |
| 9_2-10; 24D5-H; HRP00049; HRP-00052 | |
| 5F10; 9F6; 5C10 and humanized variants | US 2018/0305464 |
| thereof including 5C10H1L1; | |
| 5C10H1L2; 5C10H2L1; and 5C10H2L2 | |
| 4B6, 26F5, 21F11, 23A11, 23F11 and | WO 2017/161976 |
| 22C9; BM-GT, BM-ME, 4B6-H3L4, | |
| 4B6-H4L3, 23F11-H4L4, 23F11- | |
| H4L6, 23F11-H6L4, 23F11-H6L6, | |
| 23A11-H3L3, 23A11-H3L5, | |
| 23A11-H5L3 and 23A11-H5L5; | |
| 3C5-2G12 and humanized variants | WO 2017/196867 |
| thereof including h3C5H1-h3C5L1; | |
| h3C5H2-h3C5L2; h3C5H3-h3C5L2; | |
| h3C5H4-h3C5L2; | |
| 29E.2A3 and 24F.10C12 | US 8,552,154 |

C. Exemplary IgG4 Antibodies

In certain embodiments antibodies useful in the methods and compositions of the instant inventions (particularly anti-PD-1 antibodies and anti-B7-H1 antibodies) comprise IgG4 constant regions. Exemplary IgG4 antibody comprises the VL and VH Domains of any of the anti-PD-1 antibodies or anti-B7-H1 antibodies described above, an IgG CL Kappa Domain, and an IgG4 CH1, CH2 and CH3 Domains.

An exemplary CL Domain is IgG CL Kappa Domain. The amino acid sequence of an exemplary human CL Kappa Domain is (SEQ ID NO:30):

RTVAAPSVFI FPPSDEQLKS GTASVVCLLN
NFYPREAKVQ WKVDNALQSG NSQESVTEQD
SKDSTYSLSS TLTLSKADYE KHKVYACEVT
HQGLSSPVTK SFNRGEC

An exemplary CH1 Domain is a human IgG4 CH1 Domain, optionally lacking the C-terminal lysine residue. The amino acid sequence of an exemplary human IgG4 CH1 Domain is (SEQ ID NO:31):

ASTKGPSVFP LAPCSRSTSE STAALGCLVK
DYFPEPVTVS WNSGALTSGV HTFPAVLQSS
GLYSLSSVVT VPSSSLGTKT YTCNVDHKPS
NTKVDKRV

Such antibodies will preferably comprise an IgG4 CH1 Domain (SEQ ID NO:31) and ESKYGPPCP [symbol] CP (SEQ ID NO:32), which is an IgG4 Hinge variant comprising a stabilizing S228P substitution (as numbered by the EU index as set forth in Kabat) to reduce strand exchange.

The amino acid sequence of the CH2-CH3 Domain of an exemplary human IgG4 is (SEQ ID NO:33):

```
231 240 250 260 270 280
APEFLGGPSV FLFPPKPKDT LMISRTPEVT CVVVDVSQED PEVQFNWYVD
                  290 300 310 320 330
GVEVHNAKTK PREEQFNSTY RVVSVLTVLH QDWLNGKEYK CKVSNKGLPS
                  340 350 360 370 380
SIEKTISKAK GQPREPQVYT LPPSQEEMTK NQVSLTCLVK GFYPSDIAVE
                  390 400 410 420 430
WESNGQPENN YKTTPPVLDS DGSFFLYSRL TVDKSRWQEG NVFSCSVMHE
                  440 447
ALHNHYTQKS LSLSLG [symbol]
```

as numbered by the EU index as set forth in Kabat, wherein X is lysine (K) or is absent.

An exemplary anti-PD-1 monoclonal antibody designated “PD-1 mAb 1 IgG4” is a humanized anti-human PD-1 antibody. As indicated above, PD-1 mAb 1 comprises the VH and VL Domains of PD-1 mAb 1.

The amino acid sequence of the complete Heavy Chain of PD-1 mAb1 IgG4 is SEQ ID NO:34 (CDR_{H} residues and the S228P residue are shown underlined):

QVQLVQSGAE VKKPGASVKV SCKASGYSFT [symbol]
WVRQA PGQGLEWIG [symbol] [symbol] [symbol]
RVTI TVDKSTSTAY MELSSLRSED
TAVYYCAR [symbol] [symbol] WG QGTLVTVSSA
STKGPSVFPL APCSRSTSES TAALGCLVKD
YFPEPVTVSW NSGALTSGVH TFPAVLQSSG
LYSLSSVVTV PSSSLGTKTY TCNVDHKPSN
TKVDKRVESK YGPPCP [symbol] CPA PEFLGGPSVF
LFPPKPKDTL MISRTPEVTC VVVDVSQEDP
EVQFNWYVDG VEVHNAKTKP REEQFNSTYR
VVSVLTVLHQ DWLNGKEYKC KVSNKGLPSS
IEKTISKAKG QPREPQVYTL PPSQEEMTKN
QVSLTCLVKG FYPSDIAVEW ESNGQPENNY
KTTPPVLDSD GSFFLYSRLT VDKSRWQEGN
VFSCSVMHEA LHNHYTQKSL SLSLG

In SEQ ID NO:34, residues 1-119 correspond to the VH Domain of PD-1 mAb 1 (SEQ ID NO:26), amino acid residues 120-217 correspond to the human IgG4 CH1 Domain is (SEQ ID NO:31), amino acid residues 218-229 correspond to the human IgG4 Hinge Domain comprising the S228P substitution (SEQ ID NO:32), amino acid residues 230-245 correspond to the human IgG4 CH2-CH3 Domains (SEQ ID NO:33, wherein X is absent).

The amino acid sequence of the complete Light Chain of antibody PD-1 mAb 1 IgG4 possesses a kappa constant region and is (SEQ ID NO:35) (CDRL residues are shown underlined):

EIVLTQSPAT LSLSPGERAT LSC [symbol] [symbol]
WF QQKPGQPPKL LI [symbol]
GVPSRFSGSG SGTDFTLTIS SLEPEDFAVY
FC [symbol] [symbol] FGGGTKVEI KRTVAAPSVF
IFPPSDEQLK SGTASVVCLL NNFYPREAKV
QWKVDNALQS GNSQESVTEQ DSKDSTYSLS
STLTLSKADY EKHKVYACEV THQGLSSPVT
KSFNRGEC

In SEQ ID NO:35, amino acid residues 1-111 correspond to the VL Domain of PD-1 mAb 1 (SEQ ID NO:27), and amino acid residues 112-218 correspond to the Light Chain kappa constant region (SEQ ID NO:30).

Other exemplary anti-PD-1 antibodies having IgG4 constant regions are nivolumab, which is a human antibody, and pembrolizumab, which is a humanized antibody. Each comprise a kappa CL Domain, an IgG4 CH1 Domain, a stabilized IgG4 Hinge, and an IgG4 CH2-CH3 Domain as described above.

### IV. Pharmaceutical Compositions

The compositions of the invention include bulk drug compositions useful in the manufacture of compositions (e.g., impure or non-sterile compositions) and pharmaceutical compositions (i.e., pure and/or sterile compositions that are suitable for administration to a subject or patient), either of which can be used in the preparation of unit dosage forms. Composition, particularly pharmaceutical compositions useful in the methods of the instant invention include those comprising DART-A, and those comprising a molecule capable of binding PD-1 or a natural ligand of PD-1. Such compositions or pharmaceutical compositions may comprise a prophylactically or therapeutically effective amount of: DART-A and a pharmaceutically acceptable carrier; a PD-1 binding molecule and a pharmaceutically acceptable carrier; or a PD-1 ligand binding molecule and a pharmaceutically acceptable carrier.

The invention also encompasses pharmaceutical compositions comprising DART-A and a second therapeutic antibody (e.g., tumor specific monoclonal antibody) that is specific for a particular cancer antigen, and a pharmaceutically acceptable carrier.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of compositions of the invention are supplied either separately or mixed together in unit dosage form, for example, as a dry lyophilized powder or water-free concentrate, or as an aqueous solution in a hermetically sealed container such as a vial, an ampoule or a sachette indicating the quantity of active agent. Where the composition is to be administered by infusion, it can be dispensed with an infusion bottle, or bag containing sterile pharmaceutical grade water or saline so that the ingredients may be mixed, or diluted prior to administration. Where the composition is administered by injection, an ampoule of sterile water for injection, or saline or other diluent can be provided so that the ingredients may be mixed prior to administration.

The invention also provides a pharmaceutical pack or kit comprising one or more containers containing DART-A alone or with such pharmaceutically acceptable carrier. Additionally, one or more other prophylactic or therapeutic agents useful for the treatment of a disease can also be included in the pharmaceutical pack or kit. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The present invention provides kits that comprise DART-A and that can be used in the above methods. In such kits, the DART-A is preferably packaged in a hermetically sealed container, such as a vial, an ampoule or a sachette indicating the quantity of the molecule, and optionally including instructions for use. In one embodiment, the DART-A of such kit is supplied as a dry sterilized lyophilized powder or water-free concentrate in a hermetically sealed container and can be reconstituted, e.g., with water, saline, or other diluent to the appropriate concentration for administration to a subject. The lyophilized material should be stored at between 2° C. and 8° C. in their original container and the material should be administered within 12 hours, preferably within 6 hours, within 5 hours, within 3 hours, or within 1 hour after being reconstituted. In another embodiment, the DART-A of such kit is supplied as an aqueous solution in a hermetically sealed container and can be diluted, e.g., with water, saline, or other diluent, to the appropriate concentration for administration to a subject. The kit can further comprise one or more other prophylactic and/or therapeutic agents useful for the treatment of cancer, in one or more containers; and/or the kit can further comprise one or more cytotoxic antibodies that bind one or more cancer antigens associated with cancer. In certain embodiments, the other prophylactic or therapeutic agent is a chemotherapeutic. In other embodiments, the prophylactic or therapeutic agent is a biological or hormonal therapeutic. In other embodiments, the prophylactic or therapeutic agent is a PD-1 binding molecule. In other embodiments, the prophylactic or therapeutic agent is a PD-1 ligand binding molecule.

### V. Uses of the Compositions of the Invention

DART-A may be used to treat any disease or condition associated with or characterized by the expression of CD123. In particular, DART-A may be used to treat hematologic malignancies. Thus, without limitation, such molecules may be employed in the diagnosis or treatment of the hematologic malignancies: acute myeloid leukemia (AML), chronic myelogenous leukemia (CML), including blastic crisis of CML and Abelson oncogene associated with CML (Bcr-ABL translocation), myelodysplastic syndrome (MDS), acute B lymphoblastic leukemia (B-ALL), acute T lymphoblastic leukemia (T-ALL), chronic lymphocytic leukemia (CLL), including Richter's syndrome or Richter's transformation of call, hairy cell leukemia (HCL), blastic plasmacytoid dendritic cell neoplasm (BPDCN), non-Hodgkin's lymphoma (NHL), including mantle cell lymphoma (MCL) and small lymphocytic lymphoma (SLL), Hodgkin's lymphoma, systemic mastocytosis, and Burkitt's lymphoma. DART-A may additionally be used in the manufacture of medicaments for the treatment of the above-described conditions.

In specific embodiments, the present invention provides methods of treating AML, MDS, BPDCN, B-ALL, and T-ALL. In one specific embodiment, the present invention provides methods of treating AML.

### VI. Methods of Administration

As provided above, CD123×CD3 bispecific diabodies of the invention (e.g., DART-A) and pharmaceutical compositions of the present invention comprising the same may be provided for the treatment, prophylaxis, and amelioration of one or more symptoms associated with a hematological malignancy. In some embodiments, a CD123×CD3 bispecific diabody (or pharmaceutical composition comprising the same) may be used in combination with one or more additional therapeutic agent (e.g., therapeutic agents known to those skilled in the art for the treatment or prevention of a hematological malignancy, including but not limited to, current standard and experimental chemotherapeutic agents, hormonal agent, biological agent, immunotherapeutic agents, or agents useful for the mitigation of side effects of treatment including but not limited those described herein). In specific embodiments, a CD123×CD3 bispecific diabody (or pharmaceutical composition comprising the same) may be used in combination with a molecule capable of binding PD-1 or a natural ligand of PD-1 (or a pharmaceutical composition comprising the same).

As used herein, the term “combination” refers to the use of more than one therapeutic agent. The use of the term “combination” does not restrict the order in which therapeutic agents are administered to a subject with a disorder, nor does it mean that the agents are administered at exactly the same time, but rather it is meant that a CD123×CD3 bispecific diabody of the invention and the other agent are administered to a human patient or other mammal in a sequence and within a time interval such that the CD123×CD3 bispecific diabody of the invention and the other agent provide a desired therapeutic benefit. For example, each therapeutic agent (e.g., chemotherapeutic agent, hormonal agent or biological agent such as a molecule capable of binding PD-1) may be administered at the same time or sequentially in any order at different points in time; however, if not administered at the same time, they should be administered sufficiently close in time so as to provide the desired therapeutic or prophylactic effect. Each therapeutic agent can be administered separately, in any appropriate form and by any suitable route, e.g., one by the oral route and one parenterally, etc.

In particular, the present invention provides methods of treating a hematological malignancy comprising administering to a subject an effective amount of a CD123×CD3 bispecific diabody of the invention (e.g., DART-A), or a pharmaceutical composition comprising a CD123×CD3 bispecific diabody of the invention (e.g., DART-A). The present invention further provides methods of treating a hematological malignancy comprising administering to a subject an effective amount of a CD123×CD3 bispecific diabody of the invention (or a pharmaceutical composition comprising the same) in combination with a molecule capable of binding PD-1 or a natural ligand of PD-1 (or a pharmaceutical composition comprising the same). In a specific aspect, such compositions are substantially purified (i.e., substantially free from substances that limit its effect or produce undesired side effects). In a specific embodiment, the subject is an animal, preferably a mammal such as non-primate (e.g., bovine, equine, feline, canine, rodent, etc.) or a primate (e.g., monkey such as, a cynomolgus monkey, human, etc.). In a specific embodiment, the subject is a human.

Methods of administering a molecule of the invention include, but are not limited to, parenteral administration (e.g., intradermal, intramuscular, intraperitoneal, intravenous and subcutaneous). In a specific embodiment, the sequence-optimized CD123×CD3 bispecific diabodies of the invention (e.g., DART-A) are administered intravenously. Intravenous infusion is the preferred route of administration. In particular, CD123×CD3 bispecific diabodies of the invention are administered by continuous intravenous infusion that is mediated using a pump (“pump infusion”). Such continuous infusion may have a duration of from about 1 hour to about 24 hours per day, but will preferably have a duration of about 24 hours per day. The term “about” is intended to denote a range that is ±10% of the recited duration, i.e., such that an infusion of about 24 hours will be between 21.6 hours and 26.4 hours in duration. In certain embodiments, a continuous infusion having a duration of about 24 hours per day and will continue for a period of from about 1 day to about 21 days, or from about 1 day to about 14 days, or from about 1 day to about 7 days, or from about 1 day to about 4 days, or from about 1 day to about 2 days. It will be understood, that a continuous administration may need to be paused for short periods (for example to change supplies, adjust dosages, replenish drug supply, manage side effects, etc.). In particular, a continuous administration of a CD123×CD3 bispecific diabody of the invention may be paused to administer one or more additional therapeutic agents (e.g., a molecule capable of binding PD-1 or a natural ligand of PD-1). Such pauses are routine and are not generally considered as terminating a continuous infusion period.

In a specific embodiment, a molecule capable of binding PD-1 or a natural ligand of PD-1 of the invention (e.g., PD-1 mAb 1 IgG4) is administered intravenously. In particular, a molecule capable of binding PD-1 or a natural ligand of PD-1 is administered intermittently and is infused over about 30 minute to about 240 minutes. It will be understood, that such infusion may need to be paused for short periods (for example to change supplies, adjust dosages, replenish drug supply, manage side effects, etc.). Such pauses are routine and are not generally considered as terminating a infusion period. In certain embodiments, a continuous administration of a CD123×CD3 bispecific diabody of the invention may be paused to administer the molecule capable of binding PD-1 or a natural ligand of PD-1 of the invention.

The amount of the composition of the invention which will be effective in the treatment, prevention or amelioration of one or more symptoms associated with a disorder can be determined by standard clinical techniques. The precise dose to be employed in the formulation will also depend on the route of administration, and the seriousness of the condition, and should be decided according to the judgment of the practitioner and each patient's circumstances. Effective doses may be extrapolated from dose-response curves derived from in vitro or animal model test systems. Such dosages are may be determined based upon the body weight (kg) of the recipient subject or may be a flat dosage administered (i.e., a dose that is independent of the weight of the patient, and includes physically discrete units of the molecule to be administered. Where a weight-based dose is utilized the calculated dose will be administered based on the subject's body weight at baseline. Typically, a significant (≥10%) change in body weight from baseline or established plateau weight will prompt recalculation of dose.

As noted above, DART-A is preferably administered by continuous infusion having a duration of about 24 hours per day. Thus, dosages are preferably determined based on the amount of DART-A to be administered per day, for example, nanograms of DART-A per kilogram of body weight per day (ng/kg/day). As noted above, a molecule capable of binding PD-1 or a natural ligand of PD-1 of the invention (e.g., PD-1 mAb 1 IgG4) is optionally administered intermittently over a time period of less than a few hours. In certain embodiments, each dose is be determined based on the amount of the molecule capable of binding PD-1 or a natural ligand of PD-1 per kilogram of body weight, for example, milligrams of PD-1 mAb 1 IgG4 per kilogram of body weight (mg/kg). In other embodiments, a flat dose is administered, for example a fixed milligrams of PD-1 mAb 1 IgG irrespective of body weight. With respect to doses or dosages, the term “about” is intended to denote a range that is ±10% of a recited dose, such that for example, a weight-based dose of about 30 ng/kg/day will be between 27 ng/kg/day and 33 ng/kg/day patient weight, and a flat dose of about 200 mg will be between 180 mg and 220 mg.

In certain embodiments, DART-A is administered using 1-week (7-day) “periods” (“P”). As discussed in detail below, administration comprises an initial 7-day treatment period (the “I7DP”), which may be followed by one or more additional 7-day treatment periods (each being an “A7DP;” e.g., A7DP 1, A7DP 2, etc.). The final A7DP of a treatment cycle may be followed by one or more further 7-day treatment periods (each being an “F7DP;” e.g., F7DP 1, F7DP 2, etc.).

The term “LID-1 schema” refers to a dosing schedule comprising a one-step lead-in dosing in which DART-A is administered at 100 ng/kg/day for 4 days followed by a 3 day pause during the initial 7-day treatment period. The term “LID-2 schema” refers to a dosing schedule comprising a two-step lead-in dosing in which DART-A is administered at 30 ng/kg/day for 3 days, followed by administration at 100 ng/kg/day for the next 4 days during the initial 7-day treatment period. The term “LID-3 schema” refers to a dosing schedule comprising a multi-step lead-in dosing in which DART-A is administered using multiple step-up dose increments (more than two steps), each lasting for about 24 hours until a target dose is reached, after which DART-A is administered at the target dose for the remainder of the initial 7-day treatment period (I7DP).

In one embodiment, during the initial 7-day treatment period (I7DP), DART-A is administered using a lead-in dosing strategy incorporating multiple step-up dosing increments until reaching a target dose. In one embodiments, the starting dose is about 30 ng/kg/day and the target dose is between about 300 ng/kg/day to about 500 ng/kg/day. In one embodiment, the target dose is about 300 ng/kg/day and during the I7DP, DART-A is administered by continuous intravenous infusion: at a dosage of about 30 ng/kg/day on day 1; at a dosage of about 60 ng/kg/day on day 2; at a dosage of about 100 ng/kg/day on day 3; at a dosage of about 200 ng/kg/day on day 4; and at a dosage of about 300 ng/kg/day on days 5, 6 and 7. In another embodiment, the target dose is about 400 ng/kg/day and during the I7DP, DART-A is administered by continuous intravenous infusion: at a dosage of about 30 ng/kg/day on day 1; at a dosage of about 60 ng/kg/day on day 2; at a dosage of about 100 ng/kg/day on day 3; at a dosage of about 200 ng/kg/day on day 4; at a dosage of about 300 ng/kg/day on day 5; and at a dosage of about 400 ng/kg/day on days 6 and 7. In a further embodiment, the target dose is about 500 ng/kg/day and during the I7DP, DART-A is administered by continuous intravenous infusion: at a dosage of about 30 ng/kg/day on day 1; at a dosage of about 60 ng/kg/day on day 2; at a dosage of about 100 ng/kg/day on day 3; at a dosage of about 200 ng/kg/day on day 4; at a dosage of about 300 ng/kg/day on day 5; at a dosage of about 400 ng/kg/day on day 6; and at a dosage of about 500 ng/kg/day on day 7. The present invention specifically encompasses methods of treating a hematological malignancy comprising one I7DP according to any the any of the above embodiments.

In certain embodiments, such I7DP is followed by one or more additional 7-day treatment periods (each being an A7DP) in which DART-A is administered, by continuous intravenous infusion, at the target dose (i.e., about 300 ng/kg/day to about 500 ng/kg/day) for 7 days. In some embodiments one to twenty-three A7DPs are administered. Preferably three, A7DPs are administered. In certain embodiments, more than three A7DPs are administered, particularly where the desired response has not been observed after administration of three A7DPs. In particular embodiments, four, eight, twelve, or sixteen more A7DPs are administered (i.e., a total of seven, eleven, fifteen, nineteen, or twenty-three A7DPs). In one embodiment, the target dose is about 300 ng/kg/day and at least three A7DPs are administered. In another embodiment, the target dose is about 400 ng/kg/day and at least three A7DPs are administered. In a further embodiment, the target dose is about 500 ng/kg/day and at least three A7DPs are administered. The present invention specifically encompasses methods of treating a hematological malignancy comprising one or more A7DPs according to any the any of the above embodiments.

In certain embodiments, the last of the one or more A7DPs is followed by one or more further 7-day treatment periods (each being an F7DP) in which DART-A is administered, by continuous intravenous infusion at the target dose on a 4-day on/3-day off schedule (e.g., DART-A is provided on days 1, 2, 3 and 4 of an F7DP, but not provided on days 5, 6 and 7 of such F7DP). In particular, such F7DPs may comprise administering DART-A, by continuous intravenous infusion, at the target dose on days 1-4, with no DART-A being administered on days 5-7. In some embodiments one to twenty-four F7DPs are administered. Preferably, one, two, three, four, five, six, seven, or eight of such F7DPs are administered. In specific embodiments one to four of such F7DPs are administered. In one embodiment, the target dose is about 300 ng/kg/day and at least four F7DPs are administered. In another embodiment, the target dose is about 400 ng/kg/day and at least four F7DPs are administered. In a further embodiment, the target dose is about 500 ng/kg/day and at least four F7DPs are administered. The present invention specifically encompasses methods of treating a hematological malignancy comprising one or more F7DPs according to any the any of the above embodiments.

In certain embodiments, DART-A is administered in combination with a molecule capable of binding PD-1 or a natural ligand of PD-1 (e.g., PD-1 mAb 1 IgG4), wherein the molecule capable of binding PD-1 or a natural ligand of PD-1 is administered once every once every two weeks (“Q2W”), once every three weeks (“Q3W”), or once every four weeks (“Q4W”). In specific embodiments, the molecule capable of binding PD-1 or a natural ligand of PD-1 is administered at a weight-based dose of about 1 mg/kg to about 10 mg/kg, or at a fixed dose of about 200 to about 300 mg Q2W. In a particular embodiment, the molecule capable of binding PD-1 or a natural ligand of PD-1 is administered at a weight-based dose of about 1 mg/kg to about 3 mg/kg, Q2W. In other specific embodiments, the molecule capable of binding PD-1 or a natural ligand of PD-1 is administered at a fixed dose of about 200 to about 375 mg Q3W. In a particular embodiment, the molecule capable of binding PD-1 or a natural ligand of PD-1 is administered at a fixed dose of about 375 mg Q3W. In other specific embodiments, the molecule capable of binding PD-1 or a natural ligand of PD-1 is administered at a fixed dose of about 400 to about 500 mg Q4W. In a particular embodiment, the molecule capable of binding PD-1 or a natural ligand of PD-1 is administered at a fixed dose of about 500 mg Q4W.

In certain embodiments, the Q2W, Q3W, or Q4W administration is concurrent with one or more of the 7-day treatment periods described above in which DART-A is administered. Thus, in certain embodiments, the molecule capable of binding PD-1 or a natural ligand of PD-1 is administered Q2W, Q3W, or Q4W, wherein such administration occurs during one or more of the 7-day treatment periods provided above. In certain embodiments, the molecule capable of binding PD-1 or a natural ligand of PD-1 is administered during one or more A7DP and/or during one or more F7DP. In specific embodiments, the molecule capable of binding PD-1 or a natural ligand of PD-1 is administered on day 1 of one or more A7DP and/or on day 1 of one or more F7DP. In particular embodiments, administration of DART-A is paused during the administration of the molecule capable of binding PD-1 or a natural ligand of PD-1. In certain embodiments, the molecule capable of binding PD-1 or a natural ligand of PD-1 is administered prior to DART-A when scheduled for the same day. In certain embodiments, a first dose of the molecule capable of binding PD-1 or a natural ligand of PD-1 is administered after two 7-day treatment periods, preferably on day 15 and additional doses are administered Q2W, Q3W, or Q4W thereafter. In certain embodiments, the Q2W, Q3W, or Q4W administration of the molecule capable of binding PD-1 or a natural ligand of PD-1 continues after a last dose of DART-A is administered.

In certain embodiments, treatment is divided into 4-week (28 day) therapeutic cycles. In one embodiment, a first therapeutic cycle (“Therapeutic Cycle 1”) comprises one I7DP followed by three A7DPs to make up a 4-week Therapeutic Cycle 1. In certain embodiments, a molecule capable of binding PD-1 or a natural ligand of PD-1 is also administered during such Therapeutic Cycle 1. In one embodiment, the molecule capable of binding PD-1 or a natural ligand of PD-1 (e.g., PD-1 mAb 1 IgG4) is administered at a dose of about 1 mg/kg to about 3 mg/kg on day 15 (i.e., day 1 of the second A7DP) of such Therapeutic Cycle 1.

In certain embodiments, at least one second therapeutic cycle (each a “Therapeutic Cycle 2”) is optionally administered. The administration of at least one Therapeutic Cycle 2 is particularly preferred where the desired response has not been observed after administration Cycle 1. In a particular embodiment, each Therapeutic Cycle 2 comprises four A7DPs to make up a 4-week (28 day) Therapeutic Cycle 2. Optionally, Therapeutic Cycle 2 may be repeated to provide additional administrations of DART-A on a continuous 7-day schedule at the target dose. In certain embodiments, a molecule capable of binding PD-1 or a natural ligand of PD-1 is also administered during such Therapeutic Cycle 2. In one embodiment, the molecule capable of binding PD-1 or a natural ligand of PD-1 (e.g., PD-1 mAb 1 IgG4) is administered at a weight-based dose of about 1 mg/kg to about 3 mg/kg on day 1 and on day 15 (i.e, on day 1 of the first A7DP and on day 1 of the third A7DP) of each Therapeutic Cycle 2.

In certain embodiments, at least one third therapeutic cycle (each a “Therapeutic Cycle 3”) is administered. In particular embodiments, Therapeutic Cycle 3 comprises four F7DPs to make up a 4-week (28 day) Therapeutic Cycle 3. In certain embodiments, at least one Therapeutic Cycle 3 is administered following Therapeutic Cycle 1. In other embodiments, at least one Therapeutic Cycle 3 is administered following administration of at least one Therapeutic Cycle 2. Optionally, a Therapeutic Cycle 3 may be repeated to provide additional administrations of DART-A on a 4-day on/3-day off schedule at the target dose. In certain embodiments, a molecule capable of binding PD-1 or a natural ligand of PD-1 is also administered during such Therapeutic Cycle 3. In one embodiment, the molecule capable of binding PD-1 or a natural ligand of PD-1 (e.g., PD-1 mAb 1 IgG4) is administered at a dose of about 1 mg/kg to about 3 mg/kg on day 1 and on day 15 (i.e., on day 1 of the first F7DP and on day 1 of the third F7DP) of each Therapeutic Cycle 3.

In certain embodiments, DART-A is administered according to Therapeutic Cycle 1, followed by further administration according to Therapeutic Cycle 2, which Therapeutic Cycle 2 may be repeated, followed by further administration according to Therapeutic Cycle 3, which Therapeutic Cycle 3 may be repeated. In other embodiments, Therapeutic Cycle 2 is not administered. Accordingly, in such embodiments, DART-A is administered according to Therapeutic Cycle 1, followed by further administration according to Therapeutic Cycle 3, which Therapeutic Cycle 3 may be repeated. The present invention specifically encompasses methods of treating a hematological malignancy comprising a Therapeutic Cycle 1 according to any the any of the above embodiments. The present invention further encompasses methods of treating a hematological malignancy comprising a Therapeutic Cycle 1 according to any the any of the above embodiments followed by at least one Therapeutic Cycle 2 according to any of the above embodiments. The present invention further encompasses methods of treating a hematological malignancy comprising a Therapeutic Cycle 1 according to any the any of the above embodiments followed by at least one Therapeutic Cycle 2 according to any of the above embodiments followed by at least one Therapeutic Cycle 3 according to any of the above embodiments. An exemplary LID-3 Schema comprising Therapeutic Cycle 1, Therapeutic Cycle 2, and Therapeutic Cycle 3 is presented in Table 10B below. The present invention further encompasses methods of treating a hematological malignancy comprising a Therapeutic Cycle 1 according to any the any of the above embodiments followed by at least one Therapeutic Cycle 3 according to any of the above embodiments. An exemplary LID-3 Schema comprising Therapeutic Cycle 1, and Therapeutic Cycle 3 is presented in Table 10A below.

In specific embodiments, the molecule capable of binding PD-1 or a natural ligand of PD-1 (e.g., PD-1 mAb 1 IgG4) is administered on day 15 (i.e., day 1 of the second A7DP) of such Therapeutic Cycle 1. As provided above, additional doses of the molecule capable of binding PD-1 or a natural ligand of PD-1 are administered Q2W, Q3W, or Q4W. Accordingly, such additional doses are administered during each Therapeutic Cycle 2, each Therapeutic Cycle 3, and may continue to be administered after a last dose of DART-A is administered. In certain embodiments, DART-A is administered in combination with a molecule capable of binding PD-1 or a natural ligand of PD-1 (e.g., PD-1 mAb 1 IgG4) according to Therapeutic Cycle 1, followed by further administration according to Therapeutic Cycle 2, which Therapeutic Cycle 2 may be repeated, followed by further administration according to Therapeutic Cycle 3. An exemplary dosing schedule for administration of DART-A in combination with PD-1 mAb 1 IgG4 comprising Therapeutic Cycle 1, Therapeutic Cycle 2, and Therapeutic Cycle 3 is presented in Table 11B below. In other embodiments, Therapeutic Cycle 2 is not administered. Accordingly, in such embodiments, DART-A is administered in combination with a molecule capable of binding PD-1 or a natural ligand of PD-1 (e.g., PD-1 mAb 1 IgG4) according to Therapeutic Cycle 1, followed by further administration according to Therapeutic Cycle 3. An exemplary dosing schedule for administration of DART-A in combination with PD-1 mAb 1 IgG4 comprising Therapeutic Cycle 1, and Therapeutic Cycle 3 is presented in Table 11A below. In certain embodiments, Therapeutic Cycle 3 is followed by administration of one or more additional doses of the molecule capable of binding PD-1 or a natural ligand of PD-1 (e.g., PD-1 mAb 1 IgG4) Q2W, Q3W or Q4W. An exemplary dosing schedules for administration of DART-A in combination with PD-1 mAb 1 IgG4 comprising administering additional doses of PD-1 mAb 1 IgG4 (Q2W) after Therapeutic Cycle 3 are presented in Tables 11A-11B below.

In one embodiment, the molecule capable of binding PD-1 or a natural ligand of PD-1 comprises:
(a) a VH Domain and a VL Domain of pembrolizumab;
(b) a VH Domain and a VL Domain of nivolumab;
(c) a VH Domain and a VL Domain of cemiplimab;
(c) a VH domain and a VL domain of PD-1 mAb 1;
(d) a VH Domain and a VL Domain of atezolizumab;
(e) a VH Domain and a VL Domain of avelumab;
(f) a VH Domain and a VL Domain of durvalumab; or
(h) a VH domain and a VL domain of an antibody provided in Tables 3 or 4.

In a specific embodiment the molecule capable of binding PD-1 or a natural ligand of PD-1 is PD-1 mAb 1 IgG4. In another specific embodiment, PD-1 mAb 1 IgG4 is administered according to any of the above embodiments.

In any of the above embodiments, the molecule capable of binding PD-1 or a natural ligand of PD-1 (e.g., PD-1 mAb 1 IgG4) may be administered by intravenous infusion prior to administration of DART-A when scheduled for the same day. In any of the above embodiments, administration of DART-A may be paused while the molecule capable of binding PD-1 or a natural ligand of PD-1 (e.g., PD-1 mAb 1 IgG4) is administered. Alternatively, the molecule capable of binding PD-1 or a natural ligand of PD-1 (e.g., PD-1 mAb 1 IgG4) is administered by intravenous infusion at the same time as DART-A is being administered. Such administration may take place at different sites (e.g., DART-A via IV into a patient's left arm and the molecule capable of binding PD-1 or a natural ligand of PD-1 via IV into a patient's right arm), or in the same site (e.g., via a single IV line).

In certain embodiments, one or more additional/alternative agents are administered before, during, and/or after DART-A administration, to manage an Infusion-Related Reaction (“IRR”) and/or Cytokine Release Syndrome (“CRS”) that may occur. In particular embodiments, the administration of DART-A is paused while one or more additional/alternative agents are administered to manage an IRR and/or CRS. In certain embodiments, one or more doses of a steroid such as dexamethasone (or equivalent) may be administered to manage and IRR and/or CRS. In certain embodiments, one or more doses of an IL-6 inhibitor, IL-6R inhibitor, a TNFα inhibitor, and/or an IL-1R inhibitor, is administered to manage an IRR and/or CRS.

In a specific embodiment, one or more doses of a steroid is administered to manage IRR and/or CRS. The dose of the steroid will be selected to be sufficient to attenuate or eliminate an actual or potential IRR and/or CRS. In a specific embodiment, the steroid is administered before, during and/or after the I7DP in which DART-A is administered according to any of the above embodiments. In another specific embodiment, the steroid is administered before, during and/or after the first (or any subsequent) A7DP in which DART-A is administered according to any of the above embodiments. In another specific embodiment, the steroid is administered before, during, and/or after the first (or any subsequent) F7DP in which DART-A is administered according to any of the above embodiments. In any of the above embodiments, the administration of DART-A may be paused while one or more doses of steroid is administered to manage IRR and/or CRS.

In one embodiment, the steroid is a long duration steroid (having a half-life of about 48 hours or longer) such as dexamethasone (or equivalent). In another embodiment, the steroid is an intermediate duration steroid (having a half-life of about 12-36 hours) such as methylprednisolone (or equivalent). In another embodiment, the steroid is a short duration steroid (having a half-life of about 12 hours or less) such as hydrocortisone (or equivalent). In certain embodiments, a steroid is administered (e.g., 10-20 mg dexamethasone by IV) prior to DART-A dosing (e.g., up to 30 minutes prior) followed by an additional dose during and/or after administration of DART-A (e.g., 4 mg by IV 12 hours after DART-A dosing has initiated). Steroids such as dexamethasone (or equivalent) may also be administered (e.g., 10-20 mg by IV) prior to a change in DART-A dosing (e.g., up to 30 minutes prior) followed by an additional dose after administration of a changed DART-A dose (e.g., 4 mg by IV 12 hours after DART-A dosing has initiated).

In a specific embodiment, one or more doses of an IL-6/IL-6R inhibitor is administered to manage IRR and/or CRS. The dose of the IL-6/IL-6R inhibitor will be selected to be sufficient to attenuate or eliminate an actual or potential IRR and/or CRS. In a specific embodiment, the IL-6/IL-6R inhibitor is administered before, during and/or after the I7DP in which DART-A is administered according to any of the above embodiments. In another specific embodiment, the IL-6/IL-6R inhibitor is administered before, during and/or after the first (or any subsequent) A7DP in which DART-A is administered according to any of the above embodiments. In another specific embodiment, the IL-6/IL-6R inhibitor is administered before, during, and/or after the first (or any subsequent) F7DP in which DART-A is administered according to any of the above embodiments. In any of the above embodiments, the administration of DART-A may be paused while one or more doses of an IL-6/IL-6R inhibitor is administered to manage IRR and/or CRS.

In one embodiment, the IL-6/IL-6R inhibitor is an anti-IL-6 or anti-IL-6R antibody, for example, tocilizumab (ACTEMRA®; DrugBank Accession No. DB06273), siltuximab (SYLVANT®; DrugBank Accession No. DB09036), or clazakizumab (DrugBank Accession No. DB12849) (see, Lee, D. W. et al. (2014) “Current Concepts In The Diagnosis And Management Of Cytokine Release Syndrome,” Blood 124(2):188-195; Shimabukuro-Vornhagen, A. et al. (2018) “Cytokine Release Syndrome,” J. ImmunoTher. Canc. 656, pages 1-14).

In one embodiment, the IL-6/IL-6R inhibitor is tocilizumab, and is administered, for example, by intravenous infusion at a dose of from about 4 mg/kg to about 12 mg/kg, and particularly at a dose of from about 4 mg/kg to about 8 mg/kg. In another embodiment, the IL-6/IL-6R inhibitor is siltuximab, and is administered, for example, by intravenous infusion at a dose of from about 1 mg/kg to about 11 mg/kg, and particularly at a dose of about 11 mg/kg.

In specific embodiments, one or more doses of a TNFα inhibitor is administered to manage IRR and/or CRS. The dose of the TNFα inhibitor will be selected to be sufficient to attenuate or eliminate an actual or potential IRR and/or CRS. In a specific embodiment, the TNFα inhibitor is administered before, during, and/or after the I7DP in which DART-A is administered according to any the any of the above embodiments. In another specific embodiment, the TNFα inhibitor is administered before, during, and/or after the first (or any subsequent) A7DP in which DART-A is administered according to any of the above embodiments. In another specific embodiment, the TNFα inhibitor is administered before, during, and/or after the first (or any subsequent) F7DP in which DART-A is administered according to any of the above embodiments. In any of the above embodiments, the administration of DART-A may be paused while one or more doses of a TNFα inhibitor is administered to manage IRR and/or CRS.

In one embodiment, the TNFα inhibitor is an anti-TNFα antibody, for example, adalimumab (HUMIRA®) or a biosimilar thereof (e.g., adalimumab-atto (AMJEVITA®) (Scheinfeld, N. (2003) “Adalimumab (HUMIRA): A Review,” J. Drugs Dermatol. 2(4):375-377; DrugBank Accession No. DB00051); certolizumab pegol (CIMZIA®) or a biosimilar thereof (Goel, N. et al. (2010) “Certolizumab pegol” MAbs. 2(2):137-147; DrugBank Accession No. DB08904); golimumab (SIMPONI®) or a biosimilar thereof (Mazumdar, S. et al. (2009) “Golimumab,” mAbs. 1(5):422-431; DrugBank Accession No. DB06674), infliximab (REMICADE®) or a biosimilar thereof (e.g., INFLECTRA®, SB2 etc. (Smolen, J. S. (2011) “Infliximab: 12 Years Of Experience,” Arthritis Res. Ther. 13(Suppl 1:S2) pages 1-18; Lamb, Y. N. (2017) “SB2: An Infliximab Biosimilar,” BioDrugs. 31(5):461-464); DrugBank Accession No. DB00065), or is a TNFα-blocking receptor fusion protein, for example, etanercept (ENBREL®) or a biosimilar thereof (e.g., BENEPALI®, etanercept-szzs (EREIZI®), GP2015, etc. (Deeks, E. D. (2017) “GP2015: An Etanercept Biosimilar,” Biodrugs 31:555-558; Cantini, F. et al. (2018) “Focus On Biosimilar Etanercept Bioequivalence And Interchangeability,” Biologics: Targets and Therapy 2018:12 87-95; DrugBank Accession No. DB00005).

In one embodiment, the TNFα inhibitor used is adalimumab or a biosimilar thereof, and is administered, for example, by subcutaneous injection at a dose of about 40 mg or at a dose of about 80 mg. In one embodiment, the TNFα inhibitor is certolizumab pegol, or a biosimilar thereof, and is administered, for example, by subcutaneous injection at a dose of about 200 mg. In one embodiment, the TNFα inhibitor is golimumab, or a biosimilar thereof, and is administered, for example, by subcutaneous injection at a dose of from about 50 mg to about 100 mg, or is administered, for example, by intravenous injection at a dose of about 50 mg. In one embodiment, the TNFα inhibitor is infliximab or a biosimilar thereof, and is administered, for example, by intravenous infusion at a dose of about 100 mg or about 5 mg/kg body weight. In one embodiment, the TNFα inhibitor is etanercept or a biosimilar thereof, and is administered, for example, by subcutaneous injection at a dose of from about 25 mg to about 50 mg.

In specific embodiments, one or more doses of an IL-1R-based inhibitors (e.g., anakinra (KINERET®; DrugBank Accession No. DB00026), is administered to manage IRR and/or CRS. The dose of the IL-1R-based inhibitor will be selected to be sufficient to attenuate or eliminate an actual or potential IRR and/or CRS. In a specific embodiment, the IL-1R-based inhibitor is administered before, during, and/or after the I7DP in which DART-A is administered according to any of the above embodiments. In another specific embodiment, the IL-1R-based inhibitor is administered before, during and/or after the first (or any subsequent) A7DP in which DART-A is administered according to any of the above embodiments. In another specific embodiment, the IL-1R-based inhibitor is administered before, during, and/or after the first (or any subsequent) F7DP in which DART-A is administered according to any of the above embodiments. In any of the above embodiments, the administration of DART-A may be paused while one or more doses of an IL-1R inhibitor is administered to manage IRR and/or CRS.

In one embodiment, the IL-1R inhibitor is anakinra, and is administered, for example, by subcutaneous injection at a dose of from about 100 mg to about 150 mg.

### VII. Embodiments of the Invention

Having now generally described the invention, the same will be more readily understood through reference to the following numbered Embodiments (“E”), which are provided by way of illustration and are not intended to be limiting of the present invention unless specified:
E1. A method of treating a hematologic malignancy comprising administering a CD123×CD3 binding molecule to a subject in need thereof, wherein:
   (I) said CD123×CD3 binding molecule is a diabody consisting of a first polypeptide chain having the amino acid sequence of SEQ ID NO:21 and a second polypeptide chain having the amino acid sequence of SEQ ID NO:23; and
   (II) said method comprises an initial 7-day treatment period (I7DP), wherein:
      (A) on day 1 of said I7DP, said CD123×CD3 binding molecule is administered to said subject at a dosage of about 30 ng/kg/day by continuous intravenous infusion;
      (B) on day 2 of said I7DP, said CD123×CD3 binding molecule is administered to said subject at a dosage of about 60 ng/kg/day by continuous intravenous infusion;
      (C) on day 3 of said I7DP, said CD123×CD3 binding molecule is administered to said subject at a dosage of about 100 ng/kg/day by continuous infusion;
      (D) on day 4 of said I7DP, said CD123×CD3 binding molecule is administered to said subject at a dosage of about 200 ng/kg/day by continuous intravenous infusion;
      (E) on day 5 of said I7DP, said CD123×CD3 binding molecule is administered to said subject at a dosage of about 300 ng/kg/day by continuous intravenous infusion;
      (F) on day 6 of said I7DP, said CD123×CD3 binding molecule is administered to said subject at a dosage of from about 300 ng/kg/day to about 400 ng/kg/day by continuous intravenous infusion; and
      (G) on day 7 of said I7DP, said CD123×CD3 binding molecule is administered to said subject at a dosage of from about 300 ng/kg/day to about 500 ng/kg/day by continuous intravenous infusion.
E2. A CD123×CD3 binding molecule for use in the treatment of a hematologic malignancy of a subject, wherein:
   (I) said CD123×CD3 binding molecule is a diabody consisting of a first polypeptide chain having the amino acid sequence of SEQ ID NO:21 and a second polypeptide chain having the amino acid sequence of SEQ ID NO:23; and
   (II) said use comprises an initial 7-Day treatment period (I7DP), wherein:
      (A) on day 1 of said I7DP, said CD123×CD3 binding molecule is administered to said subject at a dosage of about 30 ng/kg/day by continuous intravenous infusion;
      (B) on day 2 of said I7DP, said CD123×CD3 binding molecule is administered to said subject at a dosage of about 60 ng/kg/day by continuous intravenous infusion;
      (C) on day 3 of said I7DP, said CD123×CD3 binding molecule is administered to said subject at a dosage of about 100 ng/kg/day by continuous infusion;
      (D) on day 4 of said I7DP, said CD123×CD3 binding molecule is administered to said subject at a dosage of about 200 ng/kg/day by continuous intravenous infusion;
      (E) on day 5 of said I7DP, said CD123×CD3 binding molecule is administered to said subject at a dosage of about 300 ng/kg/day by continuous intravenous infusion;
      (F) on day 6 of said I7DP, said CD123×CD3 binding molecule is administered to said subject at a dosage of from about 300 ng/kg/day to about 400 ng/kg/day by continuous intravenous infusion; and
      (G) on day 7 of said I7DP, said CD123×CD3 binding molecule is administered to said subject at a dosage of from about 300 ng/kg/day to about 500 ng/kg/day by continuous intravenous infusion.
E3. The method of E1, or the CD123×CD3 binding molecule for said use of E2, wherein in said method or said use comprises one or more additional 7-Day treatment periods (A7DP), wherein on days 1-7 of each of said one or more A7DP(s), said CD123×CD3 binding molecule is administered to said subject at a dosage of from about 300 ng/kg/day to about 500 ng/kg/day by continuous intravenous infusion.
E4. The method of any one of E1 or E3, or the CD123×CD3 binding molecule for said use of any one of E2 or E3, wherein on day 6, and day 7 of said I7DP, said CD123×CD3 binding molecule is administered to said subject at a dosage of about 300 ng/kg/day.
E5. The method of any one of E3 or E4, or the CD123×CD3 binding molecule for said use of any one of E3 or E4, wherein the on days 1-7 of at least one of said one or more A7DP(s), said CD123×CD3 binding molecule is administered to said subject at a dosage of about 300 ng/kg/day.
E6. The method of any one of E1 or E3, or the CD123×CD3 binding molecule for said use of any one of E2 or E3, wherein on day 6 and day 7 of said I7DP, said CD123×CD3 binding molecule is administered to said subject at a dosage of about 400 ng/kg/day.
E7. The method of any one of E3 or E6, or the CD123×CD3 binding molecule for said use of any one of E3 or E6, wherein on days 1-7 of at least one of said one or more A7DP(s), said CD123×CD3 binding molecule is administered to said subject at a dosage of about 400 ng/kg/day.
E8. The method of any one of E1 or E3, or the CD123×CD3 binding molecule for said use of any one of E2 or E3, wherein on day 6 of said I7DP, said CD123×CD3 binding molecule is administered to said subject at a dosage of about 400 ng/kg/day, and on day 7 of said I7DP, said CD123×CD3 binding molecule is administered to said subject at a dosage of about 500 ng/kg/day.
E9. The method of any one of E3 or E8, or the CD123×CD3 binding molecule for said use of any one of E3 or E8, wherein on days 1-7 of at least one of said one or more A7DP(s), said CD123×CD3 binding molecule is administered to said subject at a dosage of about 500 ng/kg/day.
E10. The method of any one of E3-E9, or the CD123×CD3 binding molecule for said use of any one of E3-E9, which comprises three of said A7DPs.

E11. The method of E10, or the CD123×CD3 binding molecule for said use of E10, which comprises and additional four, eight, twelve, sixteen, or twenty of said A7DPs.
E12. The method of any one of E3-E11, or the CD123×CD3 binding molecule for said use of any one of E3-E11, wherein at least one of said one or more A7DPs is followed by one or more further 7-day treatment periods (F7DPs), wherein on days 1-4 of each of said one or more F7DPs said CD123×CD3 binding molecule is administered to said subject, and on days 5-7 of each of said one or more F7DPs said subject is not provided with said CD123×CD3 binding molecule
E13. The method of E12, or the CD123×CD3 binding molecule for said use E12, wherein on days 1-4 of at least one of said one or more F7DPs, said CD123×CD3 binding molecule is administered to said subject at a dosage of about 300 ng/kg/day to about 500 ng/kg/day by continuous intravenous infusion.
E14. The method of E13, or the CD123×CD3 binding molecule for said use E13, wherein on days 1-4 of at least one of said one or more F7DPs, said CD123×CD3 binding molecule is administered to said subject at a dosage of about 300 ng/kg/day.
E15. The method of E13, or the CD123×CD3 binding molecule for said use of E13, wherein on days 1-4 of at least one of said one or more F7DPs, said CD123×CD3 binding molecule is administered to said subject at a dosage of about 400 ng/kg/day.
E16. The method of E13, or the CD123×CD3 binding molecule for said use of E13, wherein on days 1-4 of at least one of said one or more F7DPs, said CD123×CD3 binding molecule is administered to said subject at a dosage of about 500 ng/kg/day.
E17. The method of any one of E12-E16, or the CD123×CD3 binding molecule for said use of any one of E12-E16, which comprises four of said F7DPs.
E18. The method of E17, or the CD123×CD3 binding molecule for said use of E17, which comprises an additional four, eight, twelve, sixteen, or twenty of said F7DPs.
E19. The method of any one of E1 or E3-E18, or the CD123×CD3 binding molecule for said use of any one of E2-E18, wherein said method or use further comprises administering a molecule capable of binding PD-1 or a natural ligand of PD-1, and wherein said molecule capable of binding PD-1 comprises an epitope-binding domain of an antibody that binds PD-1, and said molecule capable of binding a natural ligand of PD-1 comprises an epitope-binding domain of an antibody that binds a natural ligand of PD-1.
E20. The method of E19 or the CD123×CD3 binding molecule for said use of E19, wherein said binding molecule capable of binding PD-1 or a natural ligand of PD-1 is administered once every two weeks (Q2W), once every three weeks (Q3W), or once every four weeks (Q4W).
E21. The method of any one of E19-E20, or the CD123×CD3 binding molecule for said use of any one of E19-E20, wherein said binding molecule capable of binding PD-1 or a natural ligand of PD-1 is administered starting on day 15.
E22. The method of E21 or the CD123×CD3 binding molecule for said use of E21, wherein said binding molecule capable of binding PD-1 or a natural ligand of PD-1 is administered Q2W starting on day 15.
E23. The method of E21 or the CD123×CD3 binding molecule for said use of E21, wherein said binding molecule capable of binding PD-1 or a natural ligand of PD-1 is administered Q3W starting on day 15.
E24. The method of E21 or the CD123×CD3 binding molecule for said use of E21, wherein said binding molecule capable of binding PD-1 or a natural ligand of PD-1 is administered Q4W starting on day 15.
E25. The method of any one of E19-E24, or the CD123×CD3 binding molecule for said use of any one of E19-E24, wherein said binding molecule capable of binding PD-1 or a natural ligand of PD-1 is administered on day 1 of one or more of said F7DPs.
E26. The method of any one of E19-E25, or the CD123×CD3 binding molecule for said use of any one of E19-E25, wherein said binding molecule capable of binding PD-1 or a natural ligand of PD-1 comprises:
   (a) a VH Domain and a VL Domain of pembrolizumab;
   (b) a VH Domain and a VL Domain of nivolumab;
   (c) a VH Domain and a VL Domain of cemiplimab;
   (c) a VH domain and a VL domain of PD-1 mAb 1;
   (d) a VH Domain and a VL Domain of atezolizumab;
   (e) a VH Domain and a VL Domain of avelumab;
   (f) a VH Domain and a VL Domain of durvalumab; or
   (h) a VH domain and a VL domain of an antibody provided in Tables 3 or 4.
E27. The method of E26, or the CD123×CD3 binding molecule for said use of E26, wherein said binding molecule capable of binding PD-1 or a natural ligand of PD-1:
   (a) comprises the VH domain and a VL domain of PD-1 mAb 1; or
   (b) is PD-1 mAb 1 IgG4.
E28. The method of any one of E19-E27, or the CD123×CD3 binding molecule for said use of any one of E19-E27, wherein said binding molecule capable of binding PD-1 or a natural ligand of PD-1 is administered at a dose of about 1 mg/kg to about 3 mg/kg.
E29. The method of any one of E19-E28, or the CD123×CD3 binding molecule for said use of any one of E19-E28, further comprising administering one or more doses of said binding molecule capable of binding PD-1 or a natural ligand of PD-1 after a last dose of said CD123×CD3 binding molecule is administered.
E30. The method of any one of E1, E3-E29, or the CD123×CD3 binding molecule for said use of any one of E2-E29, wherein said method or said use further comprises administering corticosteroid and/or an anti-IL-6 or anti-IL-6R antibody by intravenous infusion before, during, and/or after said administration of said CD123×CD3 binding molecule.
E31. The method of E30, or the CD123×CD3 binding molecule for said use of E28, wherein said corticosteroid is selected from the group consisting of dexamethasone, methylprednisolone and hydrocortisone.
E32. The method of E30, or the CD123×CD3 binding molecule for said use of E29, wherein said corticosteroid is dexamethasone.
E33. The method of E30, or the CD123×CD3 binding molecule for said use of E29, wherein said corticosteroid is methylprednisolone.
E34. The method of E30, or the CD123×CD3 binding molecule for said use of E29, wherein said corticosteroid is hydrocortisone.
E35. The method of any one of E31-E32, or the CD123×CD3 binding molecule for said use of any one of E31-E32, wherein dexamethasone is administered at a dosage of from about 10 mg to about 20 mg before administration of said CD123×CD3 binding molecule.
E36. The method of any one of E31-E32 or E35, or the CD123×CD3 binding molecule for said use of any one of E31-E32 or E35, wherein said method or use further comprises administering dexamethasone at a dosage of about 4 mg during and/or after administration of said CD123×CD3 binding molecule.
E37. The method of any one of E1 or E3-E36, or the CD123×CD3 binding molecule for said use of any one of E2-E36, wherein said method or use further comprises administering an anti-IL-6 or anti-IL-6R antibody after administration of said CD123×CD3 binding molecule.
E38. The method of E37, or the CD123×CD3 binding molecule for said use of E37, wherein said administered anti-IL-6 or anti-IL-6R antibody is tocilizumab or siltuximab.
E39. The method of E38, or the CD123×CD3 binding molecule for said use of E38, wherein said administered anti-IL-6R antibody is tocilizumab, and wherein said tocilizumab is administered at a dosage of about 4 mg/kg to about 8 mg/kg.
E40. The method of any one of E1 or E3-E39, or the CD123×CD3 binding molecule for said use of any one of E2-E39, wherein said hematologic malignancy is selected from the group consisting of: acute myeloid leukemia (AML), chronic myelogenous leukemia (CML), including blastic crisis of CML and Abelson oncogene associated with CML (Bcr-ABL translocation), myelodysplastic syndrome (MDS), acute B lymphoblastic leukemia (B-ALL), acute T lymphoblastic leukemia (T-ALL), chronic lymphocytic leukemia (CLL), including Richter's syndrome or Richter's transformation of CLL, hairy cell leukemia (HCL), blastic plasmacytoid dendritic cell neoplasm (BPDCN), non-Hodgkin's lymphoma (NHL), including mantle cell lymphoma (MCL) and small lymphocytic lymphoma (SLL), Hodgkin's lymphoma, systemic mastocytosis, and Burkitt's lymphoma.
E41. The method of E40, or the CD123×CD3 binding molecule for said use of E40, wherein said hematologic malignancy is acute myeloid leukemia.
E42. The method of E40, or the CD123×CD3 binding molecule for said use of E40, wherein said hematologic malignancy is myelodysplastic syndrome.
E43. The method of E40, or the CD123×CD3 binding molecule for said use of E40, wherein said hematologic malignancy is blastic plasmacytoid dendritic cell neoplasm.
E44. The method of E40, or the CD123×CD3 binding molecule for said use of E40, wherein said hematologic malignancy is acute T lymphoblastic leukemia.
E45. The method of E40, or the CD123×CD3 binding molecule for said use of E40, wherein said hematologic malignancy is acute B lymphoblastic leukemia.
E46. The method of any one of E1 or E3-E45, or the CD123×CD3 binding molecule for said use of any one of E2-E45, wherein said subject is a human.

### EXAMPLES

Having now generally described the invention, the same will be more readily understood through reference to the following examples, which are provided by way of illustration and are not intended to be limiting of the present invention unless specified.

### Example 1

### Activity of CD123×CD3 DART® Molecule in Primary AML Patient Samples

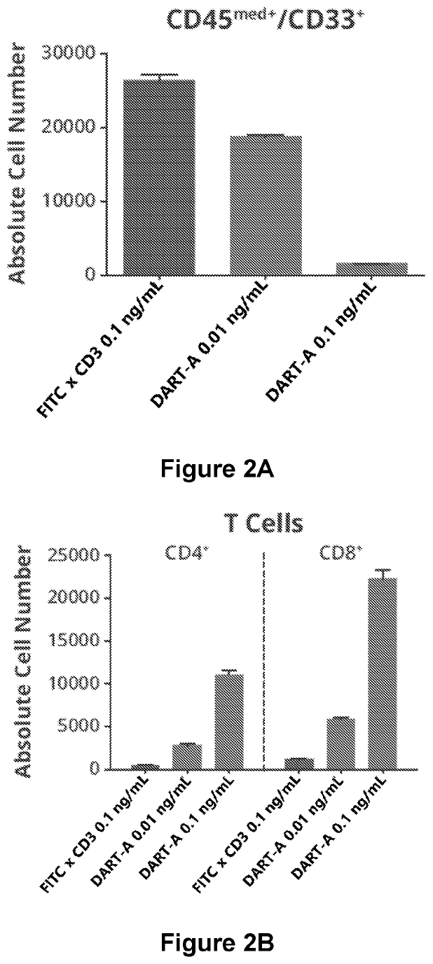

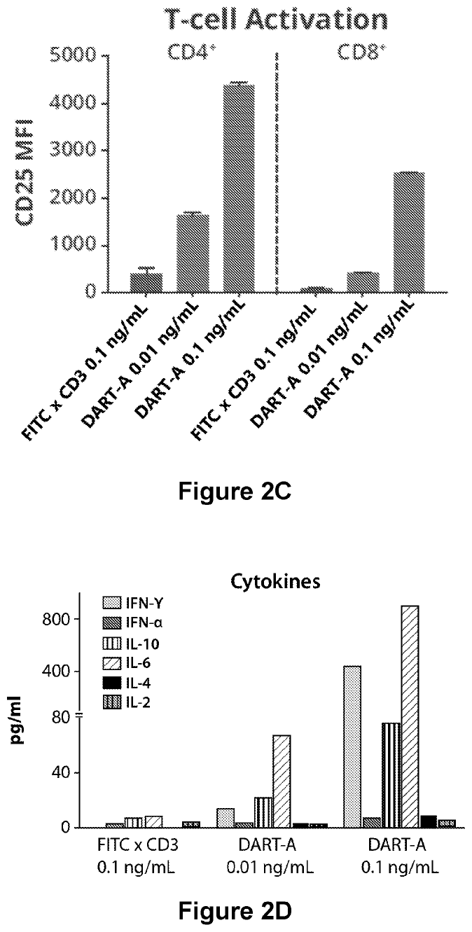

The ability of DART-A to kill CD123-expressing cells of primary AML patient samples was investigated. AML patient primary PBMCs (containing 82% blasts) were treated with a CD123×CD3 DART® molecule, a FITC×CD3 control DART® molecule, or phosphate buffered saline (PBS) for 144 hours. The E:T cell ratio was approximately 1:300 as determined from blast and T cell percentages in PBMCs at the start of the study. The absolute number of leukemic blast cells (CD45+/CD33+) is shown in FIG. 2A. The absolute numbers of T cells (CD4+ and CD8+) are shown in FIG. 2B. FIG. 2C shows T-cell activation (CD25 expression). Cytokines measured in culture supernatants are shown in FIG. 2D.

### Example 2

### Characterization of Samples Treated with DART-A

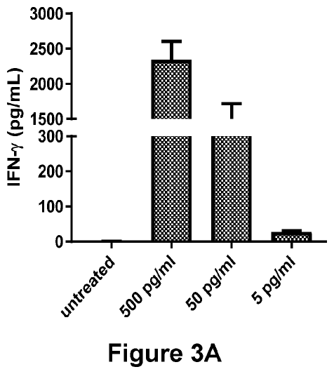

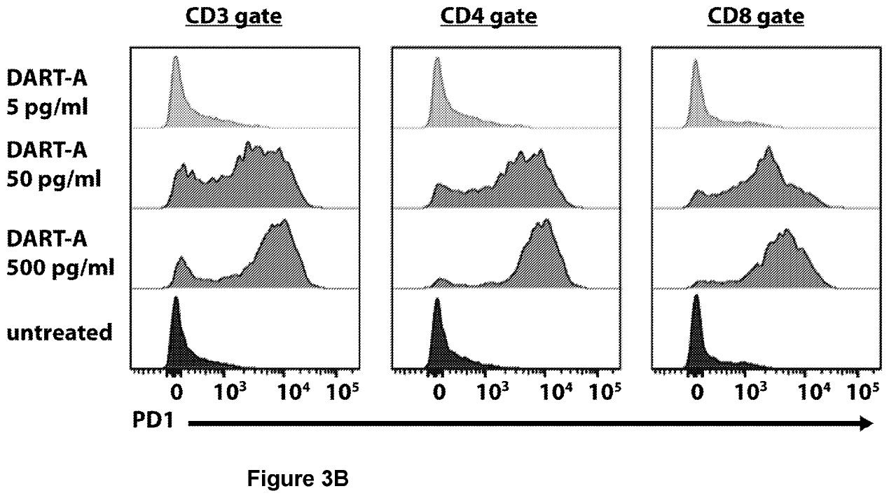

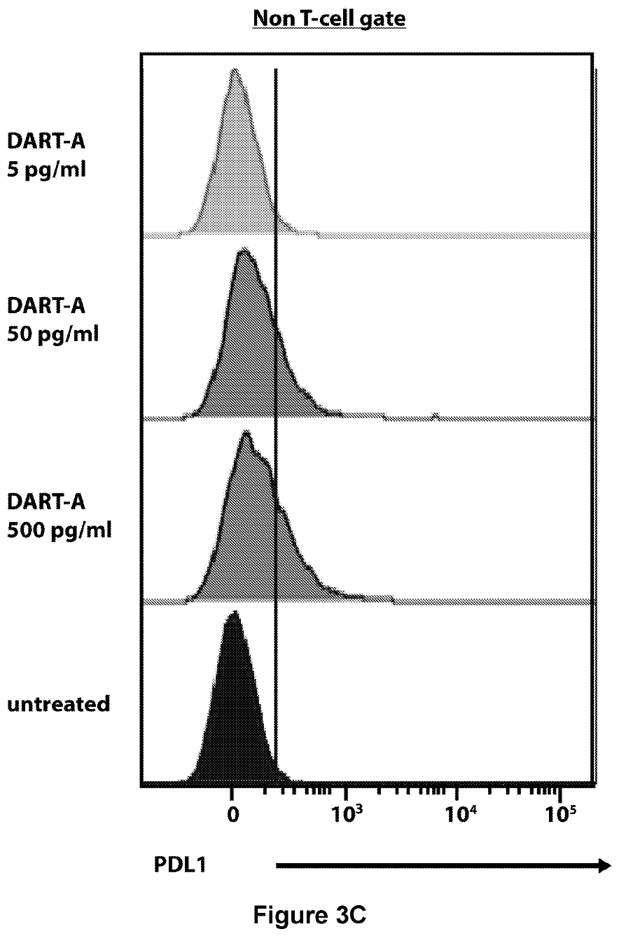

PBMC samples from AML patients were obtained from commercial sources and treated with 500, 50, or 5 pg/ml DART-A for 48 hrs. IFN-γ release was measured and the cells were stained for PD-1, PD-L1, CD3, CD4 and CD8. As shown in FIG. 3A, IFN-γ was induced in a dose dependent manner, PD-1 upregulation was observed on both CD4⁺ and CD8⁺ T-cells (FIG. 3B), and PD-L1 upregulation was observed on AML blasts (FIG. 3C) in PBMC samples, from AML patients, incubated with a DART-A molecule. IFN-γ has been reported to induce PD-L1 expression in AML blasts (Kronig, et al., (2014) “Interferon-Induced Programmed Cell Death-Ligand 1 (PD-L1/B7-H1) Expression Increases on Human Acute Myeloid Leukemia Blast Cells During Treatment,” European Journal of Haematology, 92:195-203)).

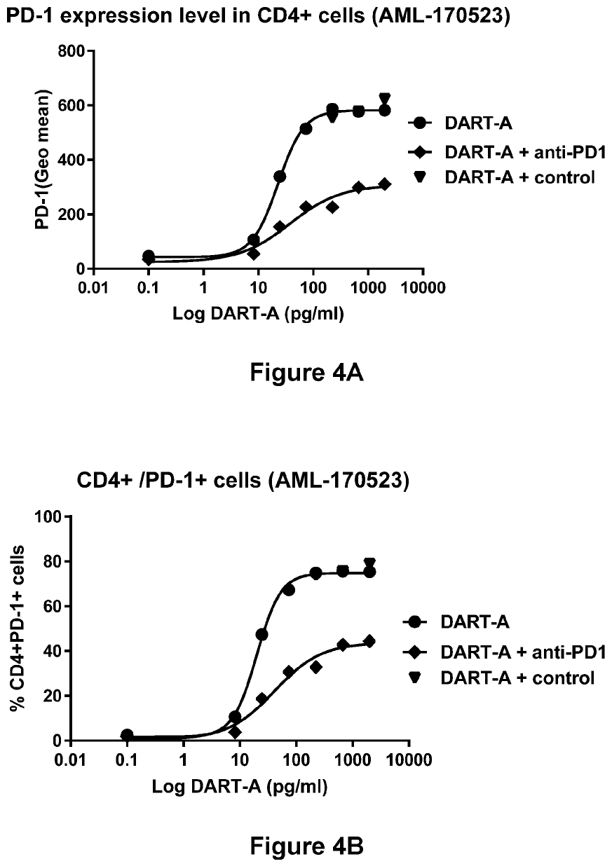

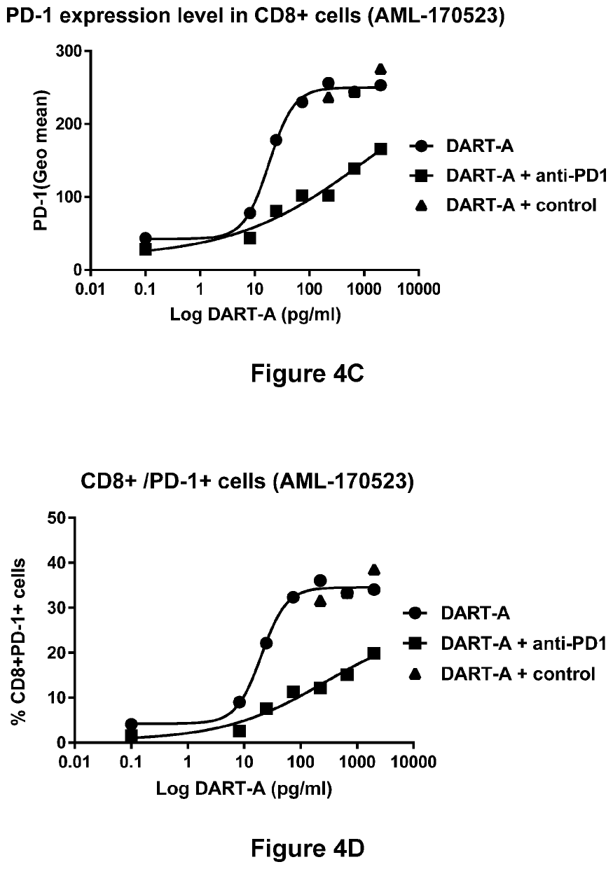

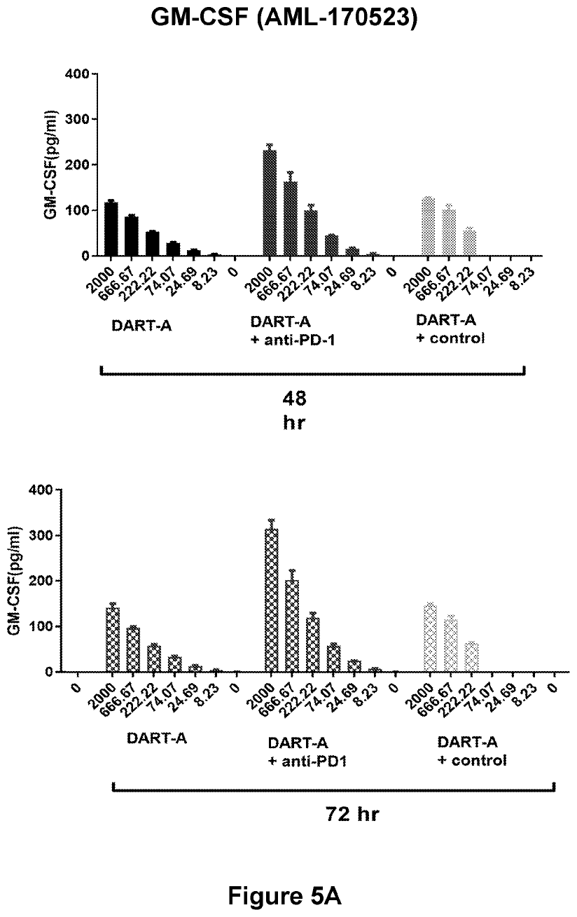

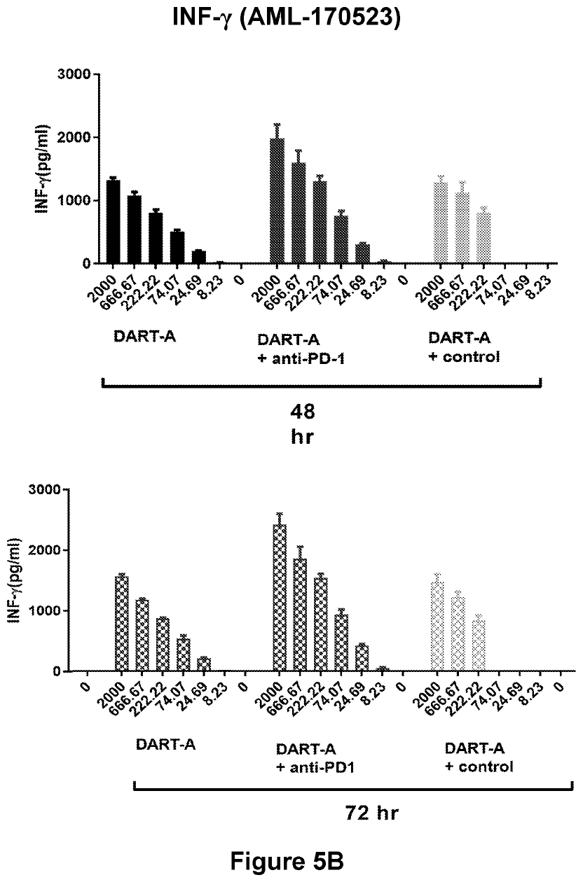

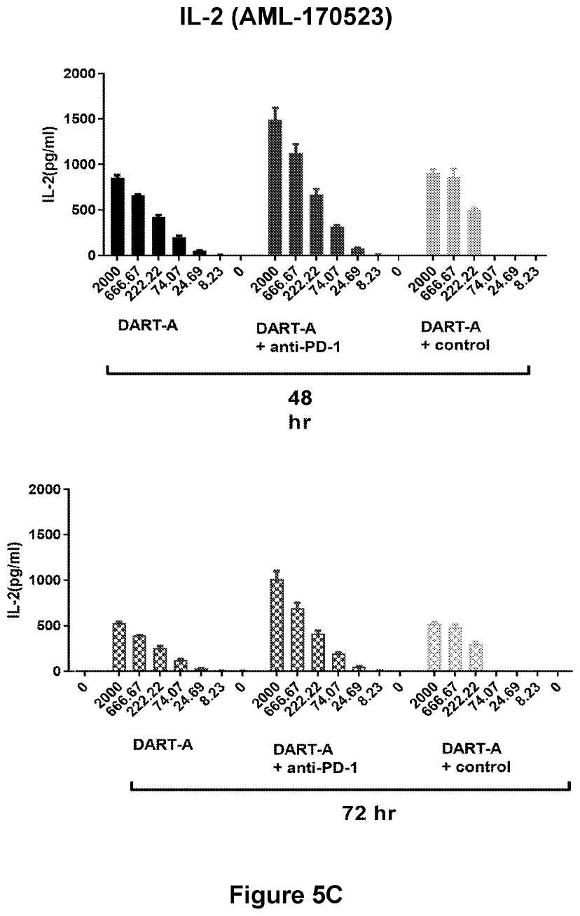

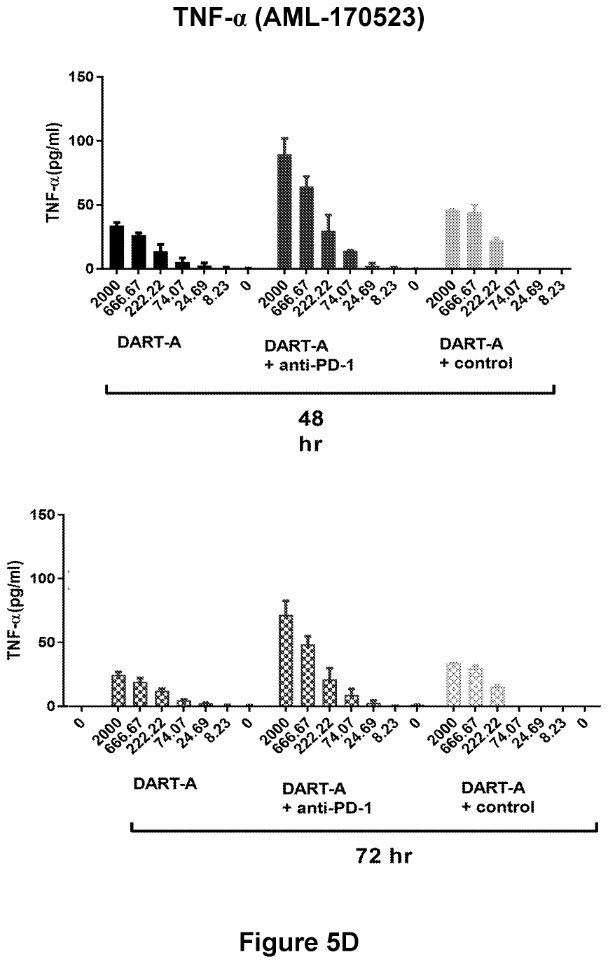

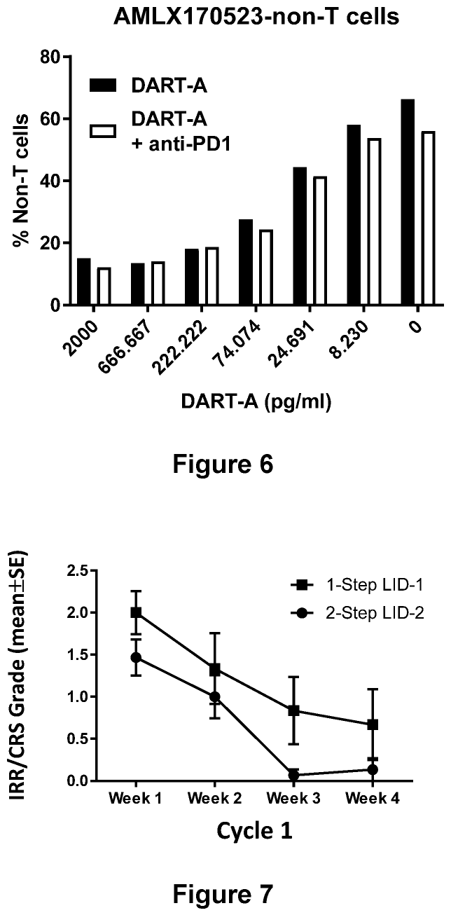

In a separate study, commercial AML-PBMC samples (in RPMI 1640/10% FBS) were incubated with a DART-A molecule (at 2000, 666.67, 222.22, 74.07, 24.69, or 8.23 pg/ml)+/−anti-PD-1 mAb (PD-1 mAb 1 IgG4; 10 μg/ml) for 48 or 72 hours. A 4420×CD3 control diabody (at 2000, 666.67, or 222.22 pg/ml) and an anti-RSV mAb were used as isotype (negative) controls. The cell surface expression of PD-1 in CD4⁺ and CD8⁺ cells was examined and the percent of cells co-expressing PD-1 and CD4⁺ or CD8⁺ was determined. In addition, cytokines were detected using BDTM cytometric bead array (CBA) kits (BD Biosciences; San Jose, Calif.) and cell killing was evaluated by examining the percent of non-T-cells. The expression of PD-1 for one such AML-PMBC sample is shown in CD4⁺ cells (total increase in CD4⁺ cells FIG. 4A, % CD4⁺PD-1⁺ cells FIG. 4B) and in CD8⁺ cells (total increase in CD8⁺ cells FIG. 4C, % CD8⁺PD-1⁺ cells FIG. 4D) and demonstrates that the enhanced expression of PD-1 on CD4⁺ and CD8⁺ cells resulting from treatment with DART-A was attenuated in the presence of the anti-PD-1 antibody checkpoint inhibitor. The data presented in FIGS. 5A-5D (summarized in Table 5), show that the release of a number of cytokines was enhanced by the combination of the DART-A molecule and the anti-PD-1 antibody checkpoint inhibitor, including GM-CSF (FIG. 5A), INF-γ (FIG. 5B), IL-2 (FIG. 5C) and TNF-α (FIG. 5D), in-vitro. These data indicate that treatment of AML cells with a DART-A molecule in combination with a molecule capable of binding PD-1 or a natural ligand of PD-1 (here an anti-PD-1 antibody) resulted in attenuated expression of PD-1 and enhanced T-cell activity. As shown in FIG. 6, at 72 hours, an enhancement of cell killing was observed for cells treated with the combination at low DART-A concentrations. In view of the increase in cytokine release, it is anticipated that the enhancement in cell killing will be greater at later time points.

TABLE 5

| | | % Increase with Anti-PD-1 Antibody | |
|---|---|---|---|
| Cytokine | DART-A (pg/mL) | 48 hr | 72 hr |
| GM-CSF | 2000 | 96.4 | 122.6 |
| | 666.67 | 90.2 | 108.4 |
| | 222.22 | 89.5 | 105.2 |
| | 74.07 | 54.5 | 75.6 |
| | 24.69 | 29.4 | 81.4 |
| INF-γ | 2000 | 49.5 | 54.3 |
| | 666.67 | 47.1 | 57.1 |
| | 222.22 | 62.0 | 75.9 |
| | 74.07 | 49.2 | 73.6 |
| | 24.69 | 50.9 | 98.5 |
| IL-2 | 2000 | 74.8 | 92.0 |
| | 666.67 | 69.6 | 76.5 |
| | 222.22 | 58.8 | 63.8 |
| | 74.07 | 57.2 | 60.0 |
| | 24.69 | 48.7 | 64.6 |
| TNF-α | 2000 | 163.8 | 186.6 |
| | 666.67 | 143.3 | 149.0 |
| | 222.22 | 112.9 | 72.6 |

These studies indicate that DART-A treatment is associated with enhanced IFN-γ secretion, and upregulation of PD-1 expression on T-cells and PD-L1 expression by the AML blasts which may result in less susceptibility to DART-A-mediated killing. These studies further indicate that combining DART-A therapy with a molecule that binds to PD-1 or a natural ligand of PD-1, such as an anti-PD1 antibody, enhances the effect of the DART-A molecule in mediating T-cell redirected killing of CD123-expressing cancer cells. Without being bound by any particular theory, such enhancement may result from overcoming the inhibitory activity of the PD-1 checkpoint. Such combinations are particularly useful in patients having a CD123 expressing hematologic malignancy (e.g., relapsed or refractory AML, B-ALL, T-ALL, or MDS).

### Example 3

### Initial Lead-In Dosing CD123×CD3 DART Diabody in AML and MDS

Acute myeloid leukemia (AML) is characterized by the expansion of CD34+, CD38⁻ cells with high levels of CD123, the alpha chain of the interleukin 3 receptor (IL-3Rα). CD123 is highly expressed in >90% of AML patients and at least 50% of MDS patients. CD123 expression in AML blasts has been related with high-risk disease and disease progression, enabling a promising strategy of preferential ablation with CD123 targeted approach. Because AML blast and leukemic stem cells highly express CD123, which is associated with high-risk disease and disease progression whereas CD123 expression on normal hematopoietic stem cells is minimal, AML (and myelodysplastic syndrome (MDS)) are reasonable targets for CD123-based immunotherapy.

The DART-A molecule of the present invention shows potent activity to target CD123-expressing cell lines and primary AML blasts in vitro for recognition and elimination by CD3-expressing T lymphocytes as effector cells, and are capable of inhibiting the growth of leukemic cell lines in mice and depleting CD123-positive plasmacytoid dendritic cells in cynomolgus macaques, and thus provide a strategy for the preferential ablation of AML with a CD123-targeted approach.

### Single-Patient Dose Escalation

In order to determine the tolerability of patients to DART-A, a “Single-Patient Dose Escalation Study” was conducted. Single patient mini-cohorts were dosed with a continuous IV infusion (CIV) using a lead-in dosing strategy of 3 ng/kg/day, followed by 10 ng/kg/day, followed by 30 ng/kg/day, followed by 100 ng/kg/day, with each such progression in dose occurring if dose-limiting toxicity (DLT) was less than 33%. The cohorts were increased to 4 patients if adverse effects (AE)≥Grade 2. The results of this study indicated that DART-A was tolerated at all tested dosages.

### Initial Lead-in Dose Optimization

Cytokine secretion with ensuing potential for cytokine release syndrome (CRS) is inherent in T-cell activation and a limiting toxicity with T-cell redirecting therapies. In the Phase 1 study of the ability of DART-A to mediate such T-cell activation in the treatment of AML and MDS, two lead-in dose (“LID”) strategies, in conjunction with early intervention with tocilizumab (Maude, S. L. et al. (2014) “Managing Cytokine Release Syndrome Associated with Novel T Cell-Engaging Therapies.” Cancer Journal 20:119-122), were compared for their ability to mitigate CRS.

Briefly, in the first LID strategy (“LID-1 schema”), DART-A was administered at 100 ng/kg/day for 4 days followed by a 3 day pause during the initial 7-day treatment period (“LID-1”), and resumption of treatment at the cohort target dose (e.g., 300 ng/kg/day or 500 ng/kg/day) starting on Day 8. The second LID strategy (“LID-2 schema”), incorporates a two-step LID (“LID-2”) during the initial 7-day treatment period in which DART-A is administered at 30 ng/kg/day for 3 days, followed by administration at 100 ng/kg/day for the next 4 days, followed, by three additional 7-day treatment periods (each being an “A7DP”) in which DART-A is administered at the cohort target dose (e.g., 300-1000 ng/kg/day) using a continuous dosing schedule (i.e., administration of DART-A at the target dose every day of the week) during Weeks 2-4 or by administration of three further 7-day treatment periods (each being a “F7DP”) in which DART-A is administered at the cohort target dose (e.g., 300-1000 ng/kg/day) using an intermittent dosing schedule (i.e., administration of DART-A at the cohort target dose for 4 days followed by a 3 day pause in which no DART-A is administered). In particular, the LID-2 schema incorporates a two-step LID (i.e., an initial LID of 30 ng/kg/day for 3 days followed by a second LID of 100 ng/kg/day for 4 days) during Cycle 1/Week 1 (“C1W1”), to be followed by three 7-day treatment periods during with DART-A is administered at the cohort target dose (e.g., 300-1000 ng/kg/day) on either of the dosing schedules (continuous (A7DP) or intermittent (F7DP)) during Cycle 1/Week 2-Cycle 1/Week 4 (C1W2-C1W4).

In Cycle 2 (“C2”), Week 5-Week 8 (W5-W8), and beyond, patients are treated on a 4-day on/3-day off intermittent dose schedule at the target dose for a maximum of 12 cycles, with 2 cycles after a complete remission (“CR”) or an incomplete blood count recovery (“CRi”). Steroid-sparing, anti-cytokine (tocilizumab) therapy is used, if clinically indicated, to manage Cytokine Release Syndrome (“CRS”) symptoms. Disease status is assessed by International Working Group (“IWG”) criteria. Samples are collected for pharmacokinetic (“PK”), anti-drug antibody (“ADA”) and cytokine analyses, including IL-2, IL-6, IL-8, IL-10, TNFα, IFN-γ and GM-CSF. A post-treatment bone marrow biopsy may also be obtained.

TABLE 6
LID-2 schema-Intermittent Dosing

| | | | |
|---|---|---|---|
| | Treatment | | |
| Period | Week | Cycle Days | Dose |

Cycle 1

| LID-2 | 1 | Days 1-3 | 30 ng/kg/day |
|---|---|---|---|
| | | Days 4-7 | 100 ng/kg/day |
| F7DP 1 | 2 | Days 8-11 | 300-1000 ng/kg/day |
| | | Days 12-14 | no drug |
| F7DP 2 | 3 | Days 15-18 | 300-1000 ng/kg/day |
| | | Days 19-21 | no drug |
| F7DP 3 | 4 | Days 22-25 | 300-1000 ng/kg/day |
| | | Days 26-28 | no drug |
| Cycle 2 | | | |
| (Cycle 2 may be repeated-up to 12 times) | | | |
| F7DP 1 | 5 | Days 1-3 | 300-1000 ng/kg/day |
| | | Days 4-7 | no drug |
| F7DP 2 | 6 | Days 8-11 | 300-1000 ng/kg/day |
| | | Days 12-14 | no drug |
| F7DP 3 | 7 | Days 15-18 | 300-1000 ng/kg/day |
| | | Days 19-21 | no drug |
| F7DP 4 | 8 | Days 22-25 | 300-1000 ng/kg/day |
| | | Days 26-28 | no drug |

The LID-2 schema with Intermittent Dosing Schedule is summarized in Table 6, and the LID-2 schema with Continuous Dosing Schedule is summarized in Table 7.

TABLE 7
LID-2 schema-Continuous Dosing

| | | | |
|---|---|---|---|
| | Treatment | Cycle | |
| Period | Week | Days | Dose |

Cycle 1

| LID-2 | 1 | Days 1-3 | 30 ng/kg/day |
|---|---|---|---|
| | | Days 4-7 | 100 ng/kg/day |
| A7DP 1 | 2 | Days 8-14 | 300-1000 ng/kg/day |
| A7DP 2 | 3 | Days 15-21 | 300-1000 ng/kg/day |
| A7DP 3 | 4 | Days 22-28 | 300-1000 ng/kg/day |
| Cycle 2 | | | |
| (Cycle 2 may be repeated-up to 12 times) | | | |
| F7DP 1 | 5 | Days 1-3 | 300-1000 ng/kg/day |
| | | Days 4-7 | no drug |
| F7DP 2 | 6 | Days 8-11 | 300-1000 ng/kg/day |
| | | Days 12-14 | no drug |
| F7DP 3 | 7 | Days 15-18 | 300-1000 ng/kg/day |
| | | Days 19-21 | no drug |
| F7DP 4 | 8 | Days 22-25 | 300-1000 ng/kg/day |
| | | Days 26-28 | no drug |

In both the LID-2 schema with Intermittent Dosing Schedule and LID-2 schema with Continuous Dosing Schedule, treatment is continued until attainment of either (1) a complete response, (2) 1-2 cycles after the attainment of a complete response, (3) for a maximum of 12 cycles, (4) dose-limiting toxicity (“DLT”), or (5) treatment failure. CRS is preferably graded according to the Lee criteria (Lee, D. W. et al. (2014) “Current Concepts In The Diagnosis And Management Of Cytokine Release Syndrome,” Blood. 124:188-195; Shimabukuro-Vornhagen, A. et al. (2018) “Cytokine Release Syndrome,” J. ImmunoTher. Canc. 656, pages 1-14). Response (complete remission (CR), incomplete blood count recovery (Cri), partial remission (PR) or improvement in peripheral blood and bone marrow (PB/BM) AML blast count) is preferably assessed by International Working Group IWG (AML) or IPSS (MDS) criteria.

In the evaluation of lead-in dose strategies, cytokines (IL-2, IL-6, IL-8, IL-10, TNFα, IFN-γ, and GM-CSF) were measured and CRS severity was graded. Peak cytokine values during first reported CRS events, occurring within 10 days of start of first dose, were evaluated. Median peak cytokine levels were compared between patients with and without LID. Other potential CRS determinants were evaluated.

Infusion-related reaction (IRR)/CRS occurred in (76%) of patients, with most events (82%)≤Grade (Gr) 2, manageable and reversible. Among 29 patients with complete cytokine data, 68% experienced CRS within 2 days of start of DART-A therapy, and an additional 8% within 10 days of the start of DART-A therapy (14% Gr 1, 55% Gr2, and 7% Gr 3). Cytokine levels were generally higher in patients with CRS than in patients without CRS (median IL-6, 116.2 vs. 67.9 pg/mL; IL-8, 191.1, vs. 144.6 pg/mL; IL-10, 867.6, vs. 348.7 pg/mL), and were generally higher with increasing CRS grade. The use of a two-step LID (LID-2) reduced overall cytokine levels, with institution of the LID-2 in Week 1 decreasing severity by mean 0.54 grade during cycle 1 (mean CRS grade week 1, 1.16 vs. 2; week 2, 1 vs. 1.33; week 3, 0.67 vs. 0.83; week 4, 0.13 vs 0.67 LID-2 vs. LID-1, respectively). Median peak cytokine levels observed with the LID-2 were lower during Week 1 and after achieving maximum dose. Preliminary data show relation between baseline circulating T-cell number and maximum CRS grade during Week 1, with higher grade of CRS (≥2) in Week 1 associated with higher baseline levels of circulating T-cells. Other variables evaluated did not trend with CRS grade. CRS grade and frequency did not correlate with response. FIG. 7 presents an overview of the CRS grade exhibited by study participants, and show that the introduction of the 2-step LID-2 schema (30 ng/kg/day for 3 days, followed by 100 ng/kg/day for the next 4 days) prior to administration of a step-up target dose (e.g., 500 ng/kg/day) decreased CRS across the first study cycle (28 days).

Once a maximum tolerated dose (“MTDS”) or maximum administered dose (“MAD”) had been determined, dose expansion occurred with patients exhibiting relapsed/refractory (“R/R”) AML in one expansion cohort and patients with hypomethylation Failure MDS in a second expansion cohort. The enrolled additional patients were used to evaluate efficacy.

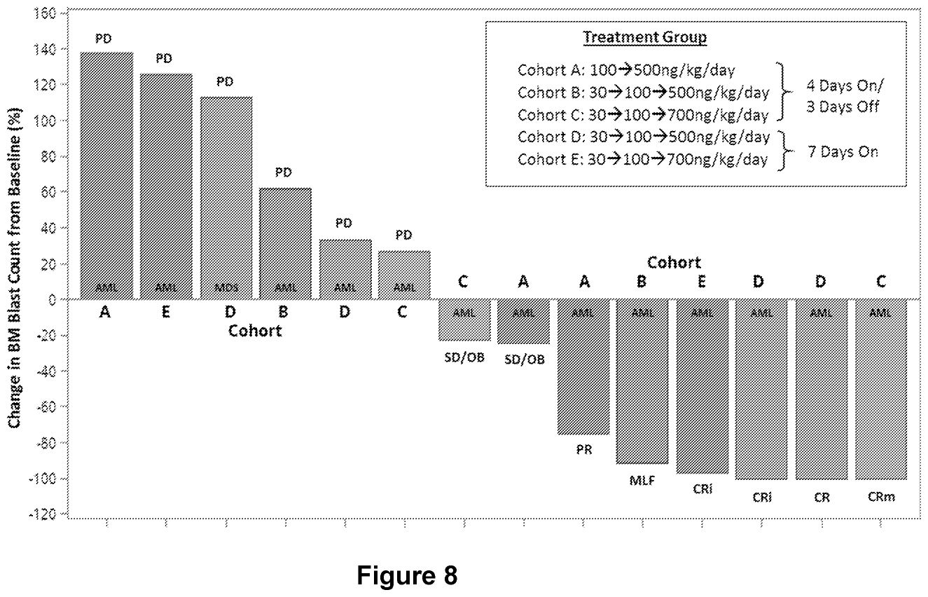

Forty-five (45) patients (median age of 64 (29-84), and 44% female) with R/R AML/MDS (89% AML and 11% MDS) were treated with DART-A. The MTDS was reached at 500 ng/kg/day. Overall, DART-A demonstrated manageable toxicity (drug-related adverse event ≥G3 were observed in 20/45 (44%) patients; infusion-related reaction/cytokine release syndrome (“IRR/CRS”) was the most common toxicity, and was observed in 34/45 (76%) patients (G3 in 6/45, 13%). The most frequent CRS symptoms were pyrexia (15), chills (10), tachycardia (10), and hypotension (4). Fourteen (14) patients treated at the threshold 500 ng/kg/day dose cohort and beyond (700 ng/kg/day dose cohort) completed at least one cycle of treatment and had a post-treatment bone marrow biopsy. Anti-leukemic activity was documented in 57% (8/14) patients, 6/14 reached IWG criteria (3 CR, 1 CRi, 1 MLF (morphologic leukemia free), 1 PR) for an objective response rate (ORR) of 43%, and 2 patients had stable disease and BM blast reduction of 20% and 25% from baseline (FIG. 8). Blast reduction occurred rapidly, often within one cycle of therapy and extended beyond DART-A discontinuation.

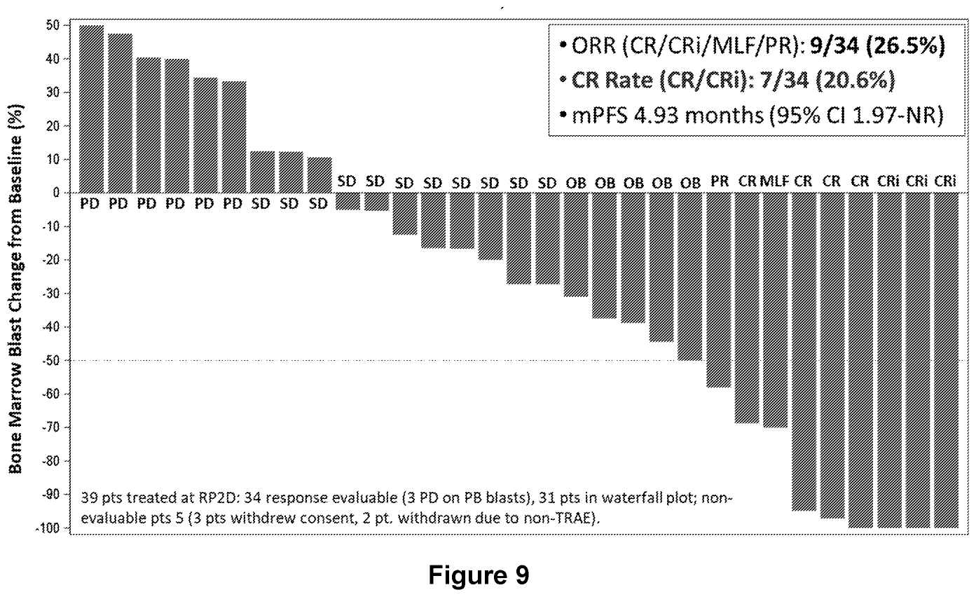

Additional patients were dosed using the LID-2 schema (30 ng/kg/day for 3 days, followed by 100 ng/kg/day for 4 days) followed by a dose of 500 ng/kg/day on Days 8-28 (Continuous Dosage Schedule (Table 7)). FIG. 9 shows DART-A anti-leukemic activity (25 patients plotted) and Table 8 shows the CRS grade by patient from 31 patients dosed using LID-2 with Continuous Dosage Schedule (Table 7).

TABLE 8
CRS Grade by Patient

| (n = 31) | N (%) |
|---|---|
| Grade 1 | 8/31 (25.8%) |
| Grade 2 | 18/31 (58.1%) |
| Grade 3 | 4/31 (12.9%) |

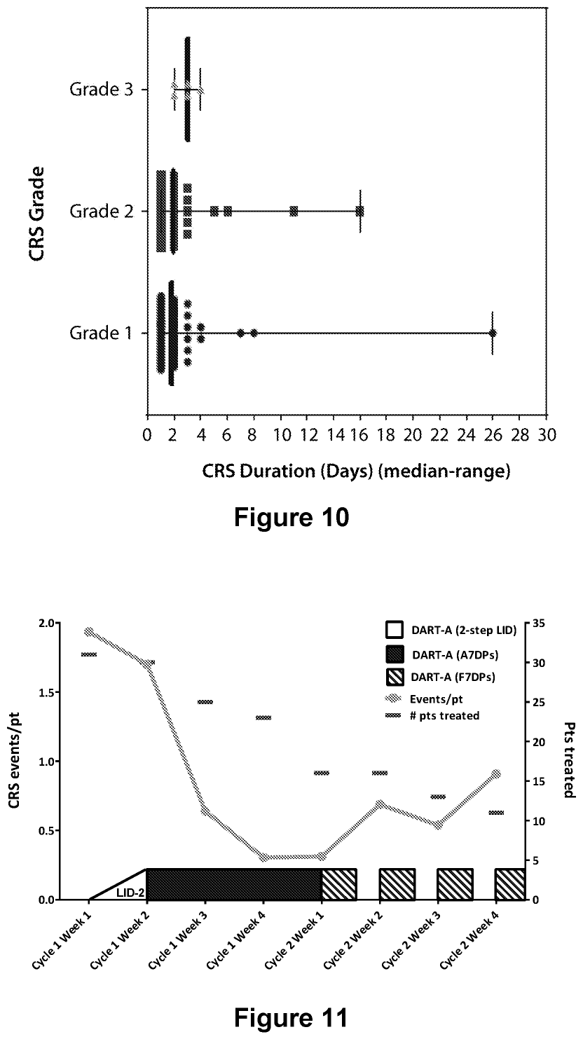

Table 9 shows the CRS grade by event from these same patients. FIG. 10 plots the CRS duration (days) for each grade and show that the median duration of CRS events was generally between 1-2.5 days (CRS Grade 1 events: 1 day; CRS Grade 2 events: 2 days; and CRS Grade 3 events: 2.5 days). However, most events (62.0%, 111/179) occurred within first week (Lead-in Dose) and during step-up to 500 ng/kg/day in the 2nd week of Cycle 1 during the continuous administration at 500 ng/kg/day (FIG. 11). Such reactions can result in treatment delays or discontinuation of treatment and can reduce the dose intensity.

TABLE 9
CRS Grade by Events

| Total = 189 | % |
|---|---|
| Grade 1 | 56.1% |
| Grade 2 | 41.3% |
| Grade 3 | 2.6% |

### Example 4

### Further Lead-In Dose Optimization

A third multi-step lead-in dosing strategy (“LID-3 schema”) is implemented for administration of DART-A to further mitigate CRS, particularly during the first two weeks of treatment.

In the multi-step LID-3 schema, DART-A is administered using multiple-step-up dose increments, each lasting for about 24 hours until the target dose (about 300 ng/kg/day to about500 ng/kg/day) is reached, after which DART-A is administered at the target dose for the remainder of the first week (i.e., the initial 7-day treatment period (I7DP)) followed by three additional 7-day treatment periods (A7DPs)) in which DART-A is administered at the target dose (e.g., about 300 ng/kg/day, about 400 ng/kg/day, or about 500 ng/kg/day) using a continuous dosing schedule. For example, where the target dose is about 500 ng/kg/day DART-A will be dosing using multiple step increments in dosing as follows: about 30 ng/kg/day, about 60 ng/kg/day, about 100 ng/kg/day, about 200 ng/kg/day, about 300 ng/kg/day, about 400 ng/kg/day each for 24 hours. On Day 7 of the I7DP, the dose will be increased to about 500 ng/kg/day and administered as a continuous infusion for three one-week A7DPs (i.e., Weeks 2-4 (days 8-28)). Together the I7DP and the first three A7DPs make up a 28-day first therapeutic cycle (Therapeutic Cycle 1). Patients that do not achieve a CR (Complete Response), CRi (Complete Response with incomplete hematological improvement), CRh (Complete Response with partial hematologic recovery), or MLF (Morphologic Leukemia-free state), after administration of Therapeutic Cycle 1 may be administered additional DART-A at the target dose using a continuous dosing schedule by administering one or more 28-day second therapeutic cycles (“Therapeutic Cycle 2”). Four A7DPs in which DART-A is administered at the cohort target dose (e.g., about 300-500 ng/kg/day) using a continuous dosing schedule make up Therapeutic Cycle 2. Therapeutic Cycle 2 may be repeated up to five times.

Thereafter patients, particularly those who achieve a CR, CRi, CRh, or MLF after administration of Therapeutic Cycle 1 alone or in combination with Therapeutic Cycle 2, are treated using a further 7-day treatment period (F7DP) in which DART-A is administered at the target dose for 4 days followed by a 3 day pause in which no DART-A is administered (i.e., on a 4-day on/3-day off schedule). Four F7DPs make up a 28-day third therapeutic cycle (Therapeutic Cycle 3). Therapeutic Cycle 3 may be repeated up to six times.

Table 10A provides the Dosing Schedule for a LID-3 schema with an I7DP having target doses of about 500 ng/kg/day, about 400 ng/kg/day, and about 300 ng/kg/day, followed by three A7DPs at the target dose (i.e., Therapeutic Cycle 1), followed by four F7DPs at the target dose (i.e., Therapeutic Cycle 3). Table 10B provides the Dosing Schedule for a LID-3 schema in which Therapeutic Cycle 1 is followed by four additional A7DPs at the target dose (i.e., Therapeutic Cycle 2), and Therapeutic Cycle 2 followed by four F7DPs (i.e, Therapeutic Cycle 3).

TABLE 10A
DART-A LID-3 Dosing Schedule‡

| | | | DART-A Target Dose ng/kg/day | | |
|---|---|---|---|---|---|
| | Treatment | | 500 | 400 | 300 |
| Period | Week | Cycle Day | ng/kg/day | ng/kg/day | ng/kg/day |

Therapeutic Cycle 1

| | | | | | |
|---|---|---|---|---|---|
| I7DP | 1 | Days 1 | 30 | 30 | 30 |
| | | Day 2 | 60 | 60 | 60 |
| | | Day 3 | 100 | 100 | 100 |
| | | Day 4 | 200 | 200 | 200 |
| | | Day 5 | 300 | 300 | 300 |
| | | Day 6 | 400 | 400 | 300 |
| | | Day 7 | 500 | 400 | 300 |
| A7DP 1 | 2 | Days 8-14 | 500 | 400 | 300 |
| A7DP 2 | 3 | Days 15-21 | 500 | 400 | 300 |
| A7DP 3 | 4 | Days 22-28 | 500 | 400 | 300 |

Therapeutic Cycle 3 (showing one Therapeutic Cycle 3,
but may include up to 6, if appropriate)

| | | | | | |
|---|---|---|---|---|---|
| F7DP 1 | 5 | Days 1-4 | 500 | 400 | 300 |
| | | Days 5-7 | no drug | no drug | no drug |
| F7DP 2 | 6 | Days 8-11 | 500 | 400 | 300 |
| | | Days 12-14 | no drug | no drug | no drug |
| F7DP 3 | 7 | Days 15-18 | 500 | 400 | 300 |
| | | Days 19-21 | no drug | no drug | no drug |
| F7DP 4 | 8 | Days 22-25 | 500 | 400 | 300 |
| | | Days 26-28 | no drug | no drug | no drug |

‡all doses ± 10%

TABLE 10B
DART-A LID-3 Dosing Schedule‡

| | | | DART-A Target Dose ng/kg/day | | |
|---|---|---|---|---|---|
| | Treatment | | 500 | 400 | 300 |
| Period | Week | Cycle Days | ng/kg/day | ng/kg/day | ng/kg/day |

Therapeutic Cycle 1

| | | | | | |
|---|---|---|---|---|---|
| I7DP | 1 | Days 1 | 30 | 30 | 30 |
| | | Day 2 | 60 | 60 | 60 |
| | | Day 3 | 100 | 100 | 100 |
| | | Day 4 | 200 | 200 | 200 |
| | | Day 5 | 300 | 300 | 300 |
| | | Day 6 | 400 | 400 | 300 |
| | | Day 7 | 500 | 400 | 300 |
| A7DP 1 | 2 | Days 8-14 | 500 | 400 | 300 |
| A7DP 2 | 3 | Days 15-21 | 500 | 400 | 300 |
| A7DP 3 | 4 | Days 22-28 | 500 | 400 | 300 |

Therapeutic Cycle 2 (showing one Therapeutic Cycle 2,
but may include up to 5, if appropriate)

| | | | | | |
|---|---|---|---|---|---|
| A7DP 4 | 5 | Days 1-7 | 500 | 400 | 300 |
| A7DP 5 | 6 | Days 8-14 | 500 | 400 | 300 |
| A7DP 6 | 7 | Days 15-21 | 500 | 400 | 300 |
| A7DP 7 | 8 | Days 22-28 | 500 | 400 | 300 |

Therapeutic Cycle 3 (showing one Therapeutic Cycle 3,
but may include up to 6, if appropriate)

| | | | | | |
|---|---|---|---|---|---|
| F7DP 1 | 9 | Days 1-4 | 500 | 400 | 300 |
| | | Days 5-7 | no drug | no drug | no drug |
| F7DP 2 | 10 | Days 8-11 | 500 | 400 | 300 |
| | | Days 12-14 | no drug | no drug | no drug |
| F7DP 3 | 11 | Days 15-18 | 500 | 400 | 300 |
| | | Days 19-21 | no drug | no drug | no drug |
| F7DP 4 | 12 | Days 22-25 | 500 | 400 | 300 |
| | | Days 26-28 | no drug | no drug | no drug |

‡all doses ± 10%

Steroids such as dexamethasone (or equivalent) may be administered (e.g., 10-20 mg by IV) prior to DART-A dosing (e.g., up to 30 minutes prior) followed by an additional dose after administration of DART-A (e.g., 4 mg by IV 12 hours after DART-A dosing has initiated). Steroids such as dexamethasone (or equivalent) may also be administered (e.g., 10-20 mg by IV) prior to a change in DART-A dosing (e.g., up to 30 minutes prior) followed by an additional dose after administration of a changed DART-A dose (e.g., 4 mg by IV 12 hours after DART-A dosing has initiated).

Steroid-sparing, anti-cytokine, particularly anti-IL-6/anti-IL-6R (tocilizumab or siltuximab) therapy is used, if clinically indicated, to manage CRS symptoms. Disease status is assessed by IWG criteria. In particular, tocilizumab may be administered (4-8 mg/kg by IV).

Other agents which may be utilized to manage CRS symptoms, particularly CRS that is refractory to anti-IL-6/anti-IL-6R treatment (e.g., tocilizumab), include further administration of corticosteroids (e.g., dexamethasone, or equivalent), such administration may be at higher dosages (e.g., doses of dexamethasone of 30 mg or greater). Anti-TNFα agents such as etanercept (or equivalent) may be employed. In particular, etanercept may be administer (e.g., 50 mg by subcutaneous injection (SC)).

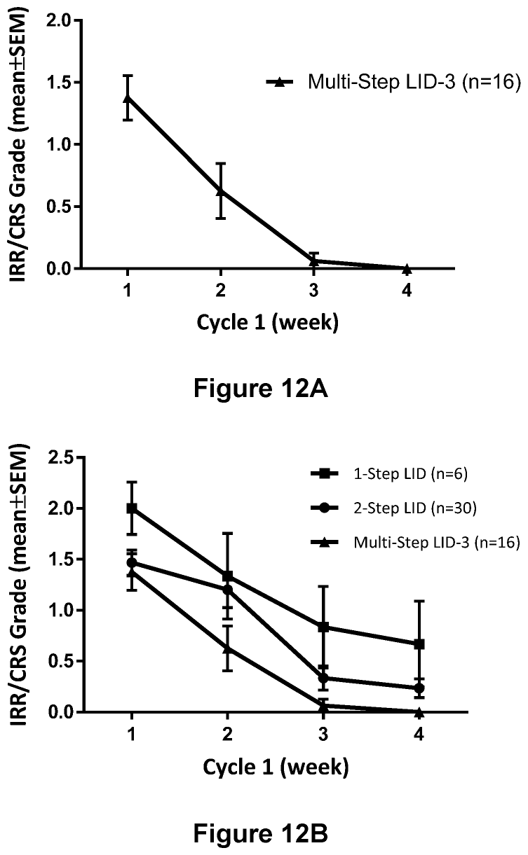

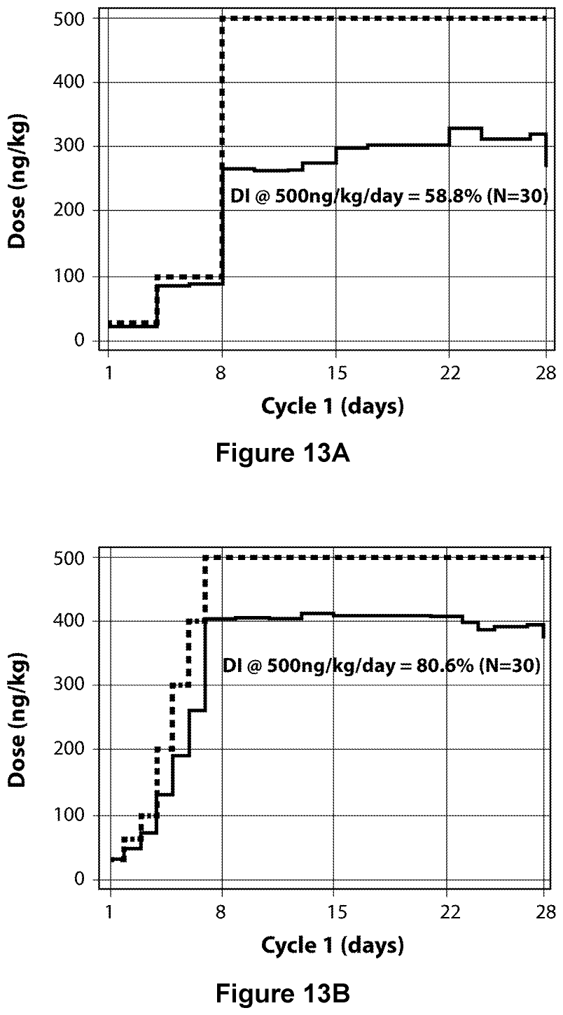

FIG. 12A presents an overview of the median IRR/CRS grade exhibited by 16 study participants during Therapeutic Cycle 1 of treatment administered DART-A using the multi-step LID-3 Schema (I7DP, target dose 500 ng/kg/day, followed by three weeks of continuous dosing at the target dose (A7DP 1-A7DP 3)). FIG. 12B compares the IRR/CRS grade data from participants administered DART-A using the multi-step LID-3 Schema, with that of subjects administered DART-A using the one-step LID (LID-1 Schema) and two-step LID (LID-2 Schema). As shown in FIGS. 12A-12B, the median IRR/CRS grade observed with the multi-step LID-3 were lower during Week 1, Week 2, and in Week 3, after achieving maximum dose as compared to those observed with the 1-step LID-1 and 2-step LID-2. In addition, as shown in FIG. 13A-13B use of multi-step LID-3 Schema improves the average dose intensity obtained by minimizing dose interruptions due to IRR and/or CRS events. Administration of DART-A using the 2-step LID-2 achieved only an average of 58.8% of the target maximum dose intensity (DI) across 30 patients during cycle 1 (FIG. 13A). In contrast, administration of DART-A using the multi-step LID-3 Schema achieved an average of 80.6% of the target maximum dose intensity (DI) across 30 patients during cycle 1 (FIG. 13B). Thus, use of the multi-step LID-3 Schema significantly increased the safety profile and the number of patients receiving the target maximum as reflected by in the increased average dose intensity.

In sum, CRS has been a limiting factor with T-cell directing therapies. The employed two-step LID-2 showed effectiveness in reducing IRR and/or CRS events and circulating cytokines over a single-step LID-1 and the multi-step LID-3 provides further improvement in limiting IRR and/or CRS events and severity. In addition, more patients receive the desired top dose intensity of 500 ng/kg/day when treated with DART-A using the multi-step LID-3 Schema. As provided in more detail below, the multi-step LID-3 dosing strategy may be adapted to include the administration of additional therapeutic agents.

### Example 5

### Combination Dosing Regimens

As provided above, DART-A therapy can be administered in combination with a molecule capable of binding PD-1 or a natural ligand of PD-1 (e.g., an anti-PD-1 antibody) to enhance the effect of the DART-A molecule in mediating T-cell redirected killing of CD123-expressing cancer cells. Accordingly, DART-A can be administered in combination with a molecule capable of binding PD-1 or a natural ligand of PD-1 such as PD-1 mAb 1 IgG4 (or other antibody described herein) for the treatment of a hematologic malignancy (e.g., relapsed or refractory AML, B-ALL, T-ALL, or MDS) according to any of the dosing schema described below.

While the following protocol details the use of DART-A in combination with the exemplary anti-PD-1 antibody “PD-1 mAb 1 IgG4,” it will be understood in view of the teachings herein that similar combination protocols may be designed using DART-A in combination with other molecules capable of binding PD-1 or a natural ligand of PD-1 (e.g., any of the anti-PD-1 antibodies or anti-B7-H1 antibodies provided herein).

In combination dosing treatment regimens, DART-A is administered using multiple-step-up dose increments, as described above, until the target dose (e.g., about 300 ng/kg/day to about 500 ng/kg/day) is reached, after which DART-A is administered at the target dose for the remainder of the first week (i.e., the initial 7-day treatment period (I7DP)) followed by three additional 7-day treatment periods (each being an “A7DP”)) in which DART-A is administered at the target dose (e.g., about 300, about 400, or about 500 ng/kg/day) using a continuous dosing schedule (i.e., administration of DART-A at the target dose each day of the week). For example, where the target dose is about 500 ng/kg/day DART-A will be dosing using multiple step increments in dosing as follows: about 30 ng/kg/day, about 60 ng/kg/day, about 100 ng/kg/day, about 200 ng/kg/day, about 300 ng/kg/day, about 400 ng/kg/day each for 24 hours. On Day 7 of the I7DP, the dose will be increased to about 500 ng/kg/day and administered as a continuous infusion for three one-week A7DPs (i.e., Weeks 2-4 (days 8-28)). Together the I7DP and the first three A7DPs make up a 28 day first therapeutic cycle (Therapeutic Cycle 1).

Patients that do not achieve a CR (Complete Response), CRi (Complete Response with incomplete hematological improvement), CRh (Complete Response with partial hematologic recovery), or MLF (Morphologic Leukemia-free state), after administration of Therapeutic Cycle 1 may be administered additional DART-A at the target dose using a continuous dosing schedule by administering one or more 28-day second therapeutic cycles (“Therapeutic Cycle 2”). Four A7DPs in which DART-A is administered at the cohort target dose (e.g., about 300 ng/kg/day to about 500 ng/kg/day) using a continuous dosing schedule make up Therapeutic Cycle 2. Therapeutic Cycle 2 may be repeated up to five times.

Thereafter patients, particularly those who achieve a CR, CRi, CRh, or MLF after administration of Therapeutic Cycle 1 alone or in combination with Therapeutic Cycle 2, are treated using a further 7-day treatment period (F7DP) in which DART-A is administered at the target dose for 4 days followed by a 3 day pause in which no DART-A is administered (i.e., on a 4-day on/3-day off schedule). Four F7DPs make up a 28-day third therapeutic cycle (Therapeutic Cycle 3).

During Therapeutic Cycles 1-3 PD-1 mAb 1 IgG4 is administered once every two weeks (“Q2W”) at a dose of about 3 mg/kg starting on day 15 (i.e., day 1 of week three). Thereafter, additional PD-1 mAb 1 IgG4 may be administered on the Q2W schedule at a dose of about 3 mg/kg. If it is determined that the maximum tolerated dose (“MTD”) is exceeded in subjects treated with 300 ng/kg/day DART-A in combination with 3 mg/kg PD-1 mAb 1 IgG4, a dose de-escalation to evaluate a lower dose of PD-1 mAb 1 IgG4 (about 1 mg/kg) in combination with 300 ng/kg/day DART-A may be utilized. Typically, PD-1 mAb 1 IgG4 is administered by intravenous infusion prior to administration of DART-A when scheduled for the same day. Thus, administration of DART-A may be paused while PD-1 mAb 1 IgG4 is administered. Alternatively, PD-1 mAb 1 IgG4 is administered by intravenous infusion at the same time as DART-A is being administered. Such administration may take place at different sites (e.g., DART-A via IV into a patient's left arm and PD-1 mAb 1 IgG4 via IV into a patient's right arm), or in the same site (e.g., via a single IV line).

Table 11A provides a Dosing Schedule for a combination dosing treatment regimen with an I7DP having target doses of about 500 ng/kg/day, about 400 ng/kg/day, and about 300 ng/kg/day, followed by three A7DPs at the target dose (i.e., Therapeutic Cycle 1), followed by four F7DPs at the target dose (i.e., Therapeutic Cycle 3). PD-1 mAb 1 IgG4 is administered once every two weeks (“Q2W”) starting on day 15 of Therapeutic Cycle 1 (i.e., day 1 of the second A7DP), on days 1 and 15 of Therapeutic Cycle 3 (i.e., day 1 of the first F7DP, and day 1 of the third F7DP) at a dose of about 3 mg/kg. As indicated, thereafter additional doses of PD-1 mAb 1 IgG4 at 3 mg/kg may be administered on the Q2W schedule. As indicated above PD-1 mAb 1 IgG4 may be administered at a de-escalation dose of 1 mg/kg.

Table 11B provides a Dosing Schedule for a combination dosing treatment regimen in which Therapeutic Cycle 1 is followed by four additional A7DPs at the target dose (i.e., Therapeutic Cycle 2), and Therapeutic Cycle 2 followed by four F7DPs (i.e., Therapeutic Cycle 3). In dosing schedules comprising a Therapeutic Cycle 2 PD-1 mAb 1 IgG4 is administered once every two weeks (“Q2W”) starting on day 15 of Therapeutic Cycle 1 (i.e., day 1 of the second A7DP), on days 1 and 15 of each Therapeutic Cycle 2 (i.e., on day 1 of the first A7DP and on day 1 of the third A7DP of each Therapeutic Cycle 2), and on days 1 and 15 of Therapeutic Cycle 3 (i.e., day 1 of the first F7DP, and day 1 of the third F7DP) at a dose of about 3 mg/kg. As indicated, thereafter additional doses of PD-1 mAb 1 IgG4 at 3 mg/kg may be administered on the Q2W schedule. As indicated above PD-1 mAb 1 IgG4 may be administered at a de-escalation dose of 1 mg/kg.

TABLE 11A
Combination Dosing Schedule‡

| | | | PD-1 mAb 1 IgG1 target dose | | |
|---|---|---|---|---|---|
| | | | 3 mg/kg | 3 mg/kg | 3 mg/kg |
| | | | DART-A Target Dose | | |
| | Treatment | | 500 | 400 | 300 |
| Period | Week | Cycle Day | ng/kg/day | ng/kg/day | ng/kg/day |

Therapeutic Cycle 1

| | | | | | |
|---|---|---|---|---|---|
| I7DP | 1 | Days 1 | 30 | 30 | 30 |
| | | Day 2 | 60 | 60 | 60 |
| | | Day 3 | 100 | 100 | 100 |
| | | Day 4 | 200 | 200 | 200 |
| | | Day 5 | 300 | 300 | 300 |
| | | Day 6 | 400 | 400 | 300 |
| | | Day 7 | 500 | 400 | 300 |
| A7DP 1 | 2 | Days 8-14 | 500 | 400 | 300 |
| A7DP 2 | 3 | Days 15†-21 | 500 | 400 | 300 |
| A7DP 3 | 4 | Days 22-28 | 500 | 400 | 300 |

Therapeutic Cycle 3

| | | | | | |
|---|---|---|---|---|---|
| F7DP 1 | 5 | Days 1**-4 | 500 | 400 | 300 |
| | | Days 5-7 | no drug | no drug | no drug |
| F7DP 2 | 6 | Days 8-11 | 500 | 400 | 300 |
| | | Days 12-14 | no drug | no drug | no drug |
| F7DP 3 | 7 | Days 15**-18 | 500 | 400 | 300 |
| | | Days 19-21 | no drug | no drug | no drug |
| F7DP 4 | 8 | Days 22-25 | 500 | 400 | 300 |
| | | Days 26-28 | no drug | no drug | no drug |

Additional Doses of PD-1 mAb 1 IgG4 at 3 mg/kg Q2W
(up to 24 doses may be administered if appropriate)
‡all doses ± 10%
†PD-1 mAb 1 IgG4 is administered on day 15 of Therapeutic Cycle 1
**PD-1 mAb 1 IgG4 is administered on days 1 and 15 of Therapeutic Cycle 3

TABLE 11B
Combination Dosing Schedule‡

| | | | PD-1 mAb 1 IgG1 target dose | | |
|---|---|---|---|---|---|
| | | | 3 mg/kg | 3 mg/kg | 3 mg/kg |
| | | | DART-A Target Dose | | |
| | Treatment | | 500 | 400 | 300 |
| Period | Week | Cycle Days | ng/kg/day | ng/kg/day | ng/kg/day |

Therapeutic Cycle 1

| | | | | | |
|---|---|---|---|---|---|
| I7DP | 1 | Days 1 | 30 | 30 | 30 |
| | | Day 2 | 60 | 60 | 60 |
| | | Day 3 | 100 | 100 | 100 |
| | | Day 4 | 200 | 200 | 200 |
| | | Day 5 | 300 | 300 | 300 |
| | | Day 6 | 400 | 400 | 300 |
| | | Day 7 | 500 | 400 | 300 |
| A7DP 1 | 2 | Days 8-14 | 500 | 400 | 300 |
| A7DP 2 | 3 | Days 15†-21 | 500 | 400 | 300 |
| A7DP 3 | 4 | Days 22-28 | 500 | 400 | 300 |

Therapeutic Cycle 2 (showing one Therapeutic Cycle 2,
but may include up to 5, if appropriate)

| | | | | | |
|---|---|---|---|---|---|
| A7DP 4 | 5 | Days 1!!-7 | 500 | 400 | 300 |
| A7DP 5 | 6 | Days 8-14 | 500 | 400 | 300 |
| A7DP 6 | 7 | Days 15!!-21 | 500 | 400 | 300 |
| A7DP 7 | 8 | Days 22-28 | 500 | 400 | 300 |

Therapeutic Cycle 3

| | | | | | |
|---|---|---|---|---|---|
| F7DP 1 | 9 | Days 1**-4 | 500 | 400 | 300 |
| | | Days 5-7 | no drug | no drug | no drug |
| F7DP 2 | 10 | Days 8-11 | 500 | 400 | 300 |
| | | Days 12-14 | no drug | no drug | no drug |
| F7DP 3 | 11 | Days 15**-18 | 500 | 400 | 300 |
| | | Days 19-21 | no drug | no drug | no drug |
| F7DP 4 | 12 | Days 22-25 | 500 | 400 | 300 |
| | | Days 26-28 | no drug | no drug | no drug |

Additional Doses of PD-1 mAb 1 IgG4 at 3 mg/kg Q2W
(up to 24 doses may be administered if appropriate)
‡all doses ± 10%
†PD-1 mAb 1 IgG4 is administered on day 15 of Therapeutic Cycle 1
!!PD-1 mAb 1 IgG4 is administered on days 1 and 15 of each Therapeutic Cycle 2
**PD-1 mAb 1 IgG4 is administered on days 1 and 15 of Therapeutic Cycle 3

In the dosing schedules provided above it will be understood that a window of about 1 day to about 3 days (i.e., ±1-3 day) in initiating a given A7DP and/or F7DP, and/or in administering a dose of PD-1 mAb 1 IgG4, may be acceptable, particularly after day 21.

Steroids such as dexamethasone (or equivalent) may be administered (e.g., 10-20 mg by IV) prior to DART-A dosing (e.g., up to 30 minutes prior) followed by an additional dose after administration of DART-A (e.g., 4 mg by IV 12 hours after DART-A dosing has initiated). Steroids such as dexamethasone (or equivalent) may also be administered (e.g., 10-20 mg by IV) prior to a change in DART-A dosing (e.g., up to 30 minutes prior) followed by an additional dose after administration of a changed DART-A dose (e.g., 4 mg by IV 12 hours after DART-A dosing has initiated).

Steroid-sparing, anti-cytokine, particularly anti-IL-6/anti-IL-6R (tocilizumab or siltuximab) therapy is used, if clinically indicated, to manage CRS symptoms. Disease status is assessed by IWG criteria. In particular, tocilizumab may be administered (4-8 mg/kg by IV).

Other agents which may be utilized to manage CRS symptoms, particularly CRS that is refractory to anti-IL-6/anti-IL-6R treatment (e.g., tocilizumab), include further administration of corticosteroids (e.g., dexamethasone, or equivalent), such administration may be at higher dosages (e.g., doses of dexamethasone of 30 mg or greater). Anti-TNFα agents such as etanercept (or equivalent) may be employed. In particular, etanercept may be administer (e.g., 50 mg by subcutaneous injection (SC)).

As provided above, it is specifically contemplated that other molecules capable of binding PD-1 or a natural ligand of PD-1 (e.g., pembrolizumab, nivolumab, avelumab, durvalumab, etc.) may be administered in combination with DART-A. In particular, such molecules may be administered in combination with DART-A wherein DART-A is administered according to Table 10A-10B or Table 11A-11B, and the molecule capable of binding PD-1 or a natural ligand of PD-1, is administered according to standard of care, or approved dosing regimens (e.g., an approved dosing regimen for pembrolizumab is intravenous administration of 200 mg Q3W).

All publications and patents mentioned in this specification are herein incorporated by reference to the same extent as if each individual publication or patent application was specifically and individually indicated to be incorporated by reference in its entirety. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A method of treating a hematologic malignancy comprising administering a CD123×CD3 binding molecule to a subject in need thereof, wherein:
(I) said CD123×CD3 binding molecule is a diabody consisting of a first polypeptide chain having the amino acid sequence of SEQ ID NO:21 and a second polypeptide chain having the amino acid sequence of SEQ ID NO:23; and
(II) said method comprises an initial 7-day treatment period (I7DP), wherein:
(A) on day 1 of said I7DP, said CD123×CD3 binding molecule is administered to said subject at a dosage of about 30 ng/kg/day by continuous intravenous infusion;
(B) on day 2 of said I7DP, said CD123×CD3 binding molecule is administered to said subject at a dosage of about 60 ng/kg/day by continuous intravenous infusion;
(C) on day 3 of said I7DP, said CD123×CD3 binding molecule is administered to said subject at a dosage of about 100 ng/kg/day by continuous infusion;
(D) on day 4 of said I7DP, said CD123×CD3 binding molecule is administered to said subject at a dosage of about 200 ng/kg/day by continuous intravenous infusion;
(E) on day 5 of said I7DP, said CD123×CD3 binding molecule is administered to said subject at a dosage of about 300 ng/kg/day by continuous intravenous infusion;
(F) on day 6 of said I7DP, said CD123×CD3 binding molecule is administered to said subject at a dosage of from about 300 ng/kg/day to about 400 ng/kg/day by continuous intravenous infusion; and
(G) on day 7 of said I7DP, said CD123×CD3 binding molecule is administered to said subject at a dosage of from about 300 ng/kg/day to about 500 ng/kg/day by continuous intravenous infusion.

2. A CD123×CD3 binding molecule for use in the treatment of a hematologic malignancy of a subject, wherein:
(I) said CD123×CD3 binding molecule is a diabody consisting of a first polypeptide chain having the amino acid sequence of SEQ ID NO:21 and a second polypeptide chain having the amino acid sequence of SEQ ID NO:23; and
(II) said use comprises an initial 7-Day treatment period (I7DP), wherein:
(A) on day 1 of said I7DP, said CD123×CD3 binding molecule is administered to said subject at a dosage of about 30 ng/kg/day by continuous intravenous infusion;
(B) on day 2 of said I7DP, said CD123×CD3 binding molecule is administered to said subject at a dosage of about 60 ng/kg/day by continuous intravenous infusion;
(C) on day 3 of said I7DP, said CD123×CD3 binding molecule is administered to said subject at a dosage of about 100 ng/kg/day by continuous infusion;
(D) on day 4 of said I7DP, said CD123×CD3 binding molecule is administered to said subject at a dosage of about 200 ng/kg/day by continuous intravenous infusion;
(E) on day 5 of said I7DP, said CD123×CD3 binding molecule is administered to said subject at a dosage of about 300 ng/kg/day by continuous intravenous infusion;
(F) on day 6 of said I7DP, said CD123×CD3 binding molecule is administered to said subject at a dosage of from about 300 ng/kg/day to about 400 ng/kg/day by continuous intravenous infusion; and
(G) on day 7 of said I7DP, said CD123×CD3 binding molecule is administered to said subject at a dosage of from about 300 ng/kg/day to about 500 ng/kg/day by continuous intravenous infusion.

3. The method of claim 1, or the CD123×CD3 binding molecule for said use of claim 2, wherein in said method or said use comprises one or more additional 7-Day treatment periods (A7DP), wherein on days 1-7 of each of said one or more A7DP(s), said CD123×CD3 binding molecule is administered to said subject at a dosage of from about 300 ng/kg/day to about 500 ng/kg/day by continuous intravenous infusion.

4. The method of any one of claim 1 or 3, or the CD123×CD3 binding molecule for said use of any one of claim 2 or 3, wherein on day 6, and day 7 of said I7DP, said CD123×CD3 binding molecule is administered to said subject at a dosage of about 300 ng/kg/day.

5. The method of any one of claim 3 or 4, or the CD123×CD3 binding molecule for said use of any one of claim 3 or 4, wherein the on days 1-7 of at least one of said one or more A7DP(s), said CD123×CD3 binding molecule is administered to said subject at a dosage of about 300 ng/kg/day.

6. The method of any one of claim 1 or 3, or the CD123×CD3 binding molecule for said use of any one of claim 2 or 3, wherein on day 6 and day 7 of said I7DP, said CD123×CD3 binding molecule is administered to said subject at a dosage of about 400 ng/kg/day.

7. The method of any one of claim 3 or 6, or the CD123×CD3 binding molecule for said use of any one of claim 3 or 6, wherein on days 1-7 of at least one of said one or more A7DP(s), said CD123×CD3 binding molecule is administered to said subject at a dosage of about 400 ng/kg/day.

8. The method of any one of claim 1 or 3, or the CD123×CD3 binding molecule for said use of any one of claim 2 or 3, wherein on day 6 of said I7DP, said CD123×CD3 binding molecule is administered to said subject at a dosage of about 400 ng/kg/day, and on day 7 of said I7DP, said CD123×CD3 binding molecule is administered to said subject at a dosage of about 500 ng/kg/day.

9. The method of any one of claim 3 or 8, or the CD123×CD3 binding molecule for said use of any one of claim 3 or 8, wherein on days 1-7 of at least one of said one or more A7DP(s), said CD123×CD3 binding molecule is administered to said subject at a dosage of about 500 ng/kg/day.

10. The method of any one of claims 3-9, or the CD123×CD3 binding molecule for said use of any one of claims 3-9, which comprises three of said A7DPs.

11. The method of claim 10, or the CD123×CD3 binding molecule for said use of claim 10, which comprises and additional four, eight, twelve, sixteen, or twenty of said A7DPs.

12. The method of any one of claims 3-11, or the CD123×CD3 binding molecule for said use of any one of claims 3-11, wherein at least one of said one or more A7DPs is followed by one or more further 7-day treatment periods (F7DPs), wherein on days 1-4 of each of said one or more F7DPs said CD123×CD3 binding molecule is administered to said subject, and on days 5-7 of each of said one or more F7DPs said subject is not provided with said CD123×CD3 binding molecule

13. The method of claim 12, or the CD123×CD3 binding molecule for said use claim 12, wherein on days 1-4 of at least one of said one or more F7DPs, said CD123×CD3 binding molecule is administered to said subject at a dosage of about 300 ng/kg/day to about 500 ng/kg/day by continuous intravenous infusion.

14. The method of claim 13, or the CD123×CD3 binding molecule for said use claim 13, wherein on days 1-4 of at least one of said one or more F7DPs, said CD123×CD3 binding molecule is administered to said subject at a dosage of about 300 ng/kg/day.

15. The method of claim 13, or the CD123×CD3 binding molecule for said use of claim 13, wherein on days 1-4 of at least one of said one or more F7DPs, said CD123×CD3 binding molecule is administered to said subject at a dosage of about 400 ng/kg/day.

16. The method of claim 13, or the CD123×CD3 binding molecule for said use of claim 13, wherein on days 1-4 of at least one of said one or more F7DPs, said CD123×CD3 binding molecule is administered to said subject at a dosage of about 500 ng/kg/day.

17. The method of any one of claims 12-16, or the CD123×CD3 binding molecule for said use of any one of claims 12-16, which comprises four of said F7DPs.

18. The method of claim 17, or the CD123×CD3 binding molecule for said use of claim 17, which comprises an additional four, eight, twelve, sixteen, or twenty of said F7DPs.

19. The method of any one of claim 1 or 3-18, or the CD123×CD3 binding molecule for said use of any one of claims 2-18, wherein said method or use further comprises administering a molecule capable of binding PD-1 or a natural ligand of PD-1, and wherein said molecule capable of binding PD-1 comprises an epitope-binding domain of an antibody that binds PD-1, and said molecule capable of binding a natural ligand of PD-1 comprises an epitope-binding domain of an antibody that binds a natural ligand of PD-1.

20. The method of claim 19 or the CD123×CD3 binding molecule for said use of claim 19, wherein said binding molecule capable of binding PD-1 or a natural ligand of PD-1 is administered once every two weeks (Q2W), once every three weeks (Q3W), or once every four weeks (Q4W).

21. The method of claim any one of claims 19-20, or the CD123×CD3 binding molecule for said use of any one of claims 19-20, wherein said binding molecule capable of binding PD-1 or a natural ligand of PD-1 is administered starting on day 15.

22. The method of claim 21 or the CD123×CD3 binding molecule for said use of claim 21, wherein said binding molecule capable of binding PD-1 or a natural ligand of PD-1 is administered 2QW starting on day 15.

23. The method of claim any one of claims 19-23, or the CD123×CD3 binding molecule for said use of any one of claims 19-23, wherein said binding molecule capable of binding PD-1 or a natural ligand of PD-1 is administered on day 1 of one or more of said F7DPs.

24. The method of any one of claims 19-23, or the CD123×CD3 binding molecule for said use of any one of claims 19-23, wherein said binding molecule capable of binding PD-1 or a natural ligand of PD-1 comprises:
(a) a VH Domain and a VL Domain of pembrolizumab;
(b) a VH Domain and a VL Domain of nivolumab;
(c) a VH Domain and a VL Domain of cemiplimab;
(c) a VH domain and a VL domain of PD-1 mAb 1;
(d) a VH Domain and a VL Domain of atezolizumab;
(e) a VH Domain and a VL Domain of avelumab;
(f) a VH Domain and a VL Domain of durvalumab; or
(h) a VH domain and a VL domain of an antibody provided in Tables 3 or 4.

25. The method of claim 24, or the CD123×CD3 binding molecule for said use of claim 24, wherein said binding molecule capable of binding PD-1 or a natural ligand of PD-1 is PD-1 mAb 1 IgG4.

26. The method of any one of claims 19-25, or the CD123×CD3 binding molecule for said use of any one of claims 19-25, wherein said binding molecule capable of binding PD-1 or a natural ligand of PD-1 is administered at a dose of about 1 mg/kg to about 3 mg/kg.

27. The method of any one of claims 19-26, or the CD123×CD3 binding molecule for said use of any one of claims 19-26, further comprising administering one or more doses of said binding molecule capable of binding PD-1 or a natural ligand of PD-1 after a last dose of said CD123×CD3 binding molecule is administered.

28. The method of any one of claims 1, 3-27, or the CD123×CD3 binding molecule for said use of any one of claims 2-27, wherein said method or said use further comprises administering a corticosteroid and/or an anti-IL-6 or anti-IL-6R antibody by intravenous infusion before, during, and/or after said administration of said CD123×CD3 binding molecule.

29. The method of any one of claim 1 or 3-28, or the CD123×CD3 binding molecule for said use of any one of claims 2-28, wherein said hematologic malignancy is selected from the group consisting of: acute myeloid leukemia (AML), chronic myelogenous leukemia (CML), including blastic crisis of CML and Abelson oncogene associated with CML (Bcr-ABL translocation), myelodysplastic syndrome (MDS), acute B lymphoblastic leukemia (B-ALL), acute T lymphoblastic leukemia (T-ALL), chronic lymphocytic leukemia (CLL), including Richter's syndrome or Richter's transformation of CLL, hairy cell leukemia (HCL), blastic plasmacytoid dendritic cell neoplasm (BPDCN), non-Hodgkin's lymphoma (NHL), including mantle cell lymphoma (MCL) and small lymphocytic lymphoma (SLL), Hodgkin's lymphoma, systemic mastocytosis, and Burkitt's lymphoma.

30. The method of claim 29, or the CD123×CD3 binding molecule for said use of claim 29, wherein said hematologic malignancy is acute myeloid leukemia.

31. The method of claim 29, or the CD123×CD3 binding molecule for said use of claim 29, wherein said hematologic malignancy is myelodysplastic syndrome.

32. The method of claim 29, or the CD123×CD3 binding molecule for said use of claim 29, wherein said hematologic malignancy is blastic plasmacytoid dendritic cell neoplasm.

33. The method of claim 29, or the CD123×CD3 binding molecule for said use of claim 29, wherein said hematologic malignancy is acute T lymphoblastic leukemia.

34. The method of claim 29, or the CD123×CD3 binding molecule for said use of claim 29, wherein said hematologic malignancy is acute B lymphoblastic leukemia.

35. The method of any one of claim 1 or 3-34, or the CD123×CD3 binding molecule for said use of any one of claims 2-34, wherein said subject is a human.
